(19) United States
(12) Patent Application Publication
(10) Pub. No.: US 2023/0181690 A1
(43) Pub. Date: Jun. 15, 2023

(54) METHODS FOR TREATING ANEMIA USING AN ACTRIIB LIGAND TRAP AND FEDRATINIB

(71) Applicant: Celgene Corporation, Summit, NJ (US)

(72) Inventors: Abderrahmane LAADEM, Belle Mead, NJ (US); Steve RITLAND, West Orange, NJ (US); Jay T. BACKSTROM, Barrington, RI (US)

(21) Appl. No.: 17/995,915

(22) PCT Filed: Apr. 12, 2021
(86) PCT No.: PCT/US2021/026816

§ 371 Date: Oct. 10, 2022

Related U.S. Application Data
(60) Provisional application No. 63/009,400, filed on Apr. 13, 2020.

(51) Int. Cl. A61K 38/17; A61K 31/506; A61P 7/06

(52) U.S. Cl. CPC A61K 38/179; A61K 31/506; A61P 7/06

(57) ABSTRACT

Provided herein are methods for treating anemia in a subject in need thereof, comprising administering to the subject an activin receptor type IIB (ActRIIB) ligand trap and administering to the subject fedratinib. Specifically, the method comprising taking a measurement of hemoglobin (Hgb) level in the subject before and after the administration.

### CROSS-REFERENCE TO RELATED APPLICATIONS

This application claims the benefit of U.S. Provisional Application No. 63/009,400, filed Apr. 13, 2020, the content of which is incorporated by reference herein in its entirety.

### REFERENCE TO SEQUENCE LISTING SUBMITTED ELECTRONICALLY

This application incorporates by reference in its entirety the Computer Readable Form (CRF) of a Sequence Listing in ASCII text format submitted herewith. The Sequence Listing text file submitted herewith, entitled 14247-487-228_SEQ_LISTING.TXT, was created on Apr. 4, 2021, and is 73,372 bytes in size.

### 1. FIELD

Provided herein are methods for treating anemia in a subject in need thereof using an activin type IIB (ActRIIB) ligand trap in combination with fedratinib. Provided herein are methods for treating anemia in a subject having myeloproliferative neoplasm- (MPN-) associated myelofibrosis using an activin type IIB (ActRIIB) in combination with fedratinib.

### 2. BACKGROUND

Myeloproliferative neoplasm-associated myelofibrosis (MF) is a serious and life-threatening disease that can present as a de novo or primary myelofibrosis (PMF) or evolve from previous polycythemia vera (post-PV MF) or essential thrombocythemia (post-ET MF) (WHO classification of tumours of haematopoietic and lymphoid tissues, ed. by Swerdlow, (IARC Press, 2008)). The disease is characterized by clonal myeloproliferation, ineffective erythropoiesis, bone marrow stromal changes, hepatosplenic extramedullary hematopoiesis, and aberrant cytokine expression (Tefferi, 2016, Am. J. Hematol. 91(12):1262-1271). Patients typically present with splenomegaly, constitutional symptoms, moderate to severe anemia, thrombocytopenia, and leukocytosis.

Primary myelofibrosis is a member of a group of Philadelphia chromosome (Ph1)-negative myeloproliferative neoplasm (MPN) which also includes PV and ET (Tefferi, 2007, Clin. Adv. Hematol. Onlco. 5:113-115). Almost all patients with PV and about one-half of patients with ET and PMF have a Janus kinase 2 (JAK2) mutation, typically JAK2V617F. Mutations in JAK2, CALR, and MPL result in activation of the JAK/signal transducers and activators of transcription (STAT) signaling pathway resulting in cell proliferation and inhibiting cell death. The result is clonal expansion (Ihle, 2007, Curr. Opin. Genet. Dev. 17(1):8-14).

Polycythemia vera (PV) and essential thrombocythemia (ET) are characterized by increased levels of red blood cells (RBCs) and platelets. However, about 10% of affected patients develop bone marrow fibrosis morphologically indistinguishable from PMF. These conditions are termed post-PV MF and post-ET MF (Campbell and Green, 2005, Hematology Am. Soc. Hematol. Educ. Program 201-208), and are clinically named MPN-associated myelofibrosis. Patients with MPN-associated myelofibrosis have similar survival prognoses to that of the PMF and about a 10% cumulative risk of transformation to acute myeloid leukemia (AML).

Approximately 70% of individuals with MF are in the intermediate-2 or high-risk categories (Gangat et al., 2011, J. Clin. Oncol. 29(4):392-397), representing the greatest unmet medical need. Symptomatic enlargement of the spleen and liver, the necessity for RBC transfusions, cachexia, and the other MF-associated symptoms result in greatly compromised quality of life in these patients (Mesa et al., Leukemia 21(9):1964-1970).

Anemia is a decrease in number of red blood cells or less than the normal quantity of hemoglobin in the blood. Anemia can also be caused by decreased oxygen-binding ability of the hemoglobin. Anemia is the most common disorder of the blood. Anemia is present at diagnosis in about two-thirds of patients with MPN-associated myelofibrosis and develops in almost all patients during the course of their disease. Overall, anemia and RBC-transfusion dependence are strong, independent prognostic variables for survival and for risk of transformation to blast phase (Passamonti et al., 2010, Blood 115(9):1703-1708; Elena et al., 2011, Haematologica 96(1):167-170). The etiology of anemia in MPN-associated myelofibrosis is complex (Barosi et al., 2010, Leuk Res. 34(9):1119-1120; Cervantes et al., 2009, Blood 113(13):2895-2901). In addition to the underlying disease, some drugs used to treat MPN-associated myelofibrosis, such as hydroxyurea (hydroxycarbamide) and JAK2 inhibitors (e.g., ruxolitinib), can exacerbate the anemia. For example, while ruxolitinib has demonstrated significant improvements in spleen size, and symptom mitigation, it is often associated with moderate to severe thrombocytopenia and anemia (Verstovsek et al., 2017, J. Hematol. Oncol. 10(1):55).

Given the current lack of safe and effective drug therapies to treat anemia in patients with MPN-associated myelofibrosis, there is significant unmet medical need for the development of new therapies that treats anemia in MPN-associated MF patients.

Two related type II receptors, ActRIIA and ActRIIB, have been identified as the type II receptors for activins (Mathews and Vale, 1991, Cell 65:973-982; Attisano et al., 1992, Cell 68: 97-108). Besides activins, ActRIIA and ActRIIB can biochemically interact with several other TGF-beta family proteins, including BMP7, Nodal, GDF8, and GDF11 (Yamashita et al., 1995, J. Cell Biol. 130:217-226; Lee and McPherron, 2001, Proc. Natl. Acad. Sci. 98:9306-9311; Yeo and Whitman, 2001, Mol. Cell 7: 949-957; Oh et al., 2002, Genes Dev. 16:2749-54). ALK4 is the primary type I receptor for activins, particularly for activin A, and ALK-7 may serve as a receptor for activins as well, particularly for activin B.

Luspatercept, an ActRIIB ligand inhibitor, has been described for treatment of various indications. See e.g. U.S. Patent Application Publication No. US 2018/0050085 A1, U.S. Pat. Nos. 8,058,229, 8,361,957, and 8,343,933.

### 3. SUMMARY

In one aspect, provided herein are methods for treating anemia in a subject in need thereof, comprising: administering to a subject an activin receptor type IIB (ActRIIB) ligand trap; and administering to the subject fedratinib or a pharmaceutically acceptable salt and/or hydrate thereof.

In another aspect, also provided herein are methods for treating anemia in a subject in need thereof, comprising: (a) taking a first measurement of hemoglobin (Hgb) level in a subject; (b) administering to the subject an initial dose of an ActRIIB ligand trap; (c) administering to the subject fedratinib or a pharmaceutically acceptable salt and/or hydrate thereof; (d) taking a second measurement of hemoglobin (Hgb) level in the subject at the end of a first period of time after the administration of the initial dose of an ActRIIB ligand trap; and (e) administering to the subject a subsequent dose of the ActRIIB ligand trap based on the second measurement of hemoglobin (Hgb) level as compared to the first measurement of hemoglobin (Hgb) level, or based on number of red blood cell transfusion that the subject received during the first period of time.

In some embodiments, the ActRIIB ligand trap and fedratinib or pharmaceutically acceptable salt or hydrate thereof are concomitantly administered.

In some embodiments, the co-administration of the ActRIIB ligand trap and fedratinib or pharmaceutically acceptable salt or hydrate thereof is pharmaceutically effective to treat anemia.

In some embodiments, the subject is a subject is diagnosed with myelofibrosis.

In some embodiments, the myelofibrosis is a myeloproliferative neoplasms (MPN)-associated myelofibrosis.

In some embodiments, the myelofibrosis is intermediate or High-Risk Primary Myelofibrosis (PMF), Post-Polycythemia Vera Myelofibrosis (post-PV MF), or Post-Essential Thrombocythemia Myelofibrosis (post-ET MF).

In some embodiments, the myelofibrosis is intermediate or high-risk primary myelofibrosis (PMF).

In some embodiments, the myelofibrosis is intermediate or high-risk post-polycythemia vera myelofibrosis (post-PV MF).

In some embodiments, the myelofibrosis is intermediate or high-risk post-essential thrombocythemia myelofibrosis (post-ET MF).

In some embodiments, the subject is a human.

In some embodiments, the anemia is associated with myeloproliferative neoplasms (MPN)-associated myelofibrosis.

In some embodiments, the subject is red blood cell (RBC) transfusion dependent or non-transfusion dependent.

In some embodiments, the subject is RBC transfusion dependent.

In some embodiments, the subject has received 4 to 12 RBC units in RBC transfusion within 84 days prior to administering of the ActRIIB ligand trap.

In some embodiments, the subject has a hemoglobin (Hgb) level of less than 11.5 g/dL without red blood cell (RBC) transfusion.

In some embodiments, the subject is non-transfusion dependent.

In some embodiments, the subject has received less than 4 red blood cell (RBC) units in RBC transfusion within 84 days prior to administering of the ActRIIB ligand trap.

In some embodiments, the subject has a hemoglobin (Hgb) level of less than 9.5 g/dL.

In some embodiments, the subject has treated with fedratinib for at least 8 weeks, at least 16 weeks, at least 24 weeks, at least 32 weeks, or at least 40 weeks prior to administration of the initial dose of the ActRIIB ligand trap and concomitantly administration of fedratinib or pharmaceutically acceptable salt or hydrate thereof.

In some embodiments, the subject has been previously treated with ruxolitinib.

In some embodiments, the fedratinib or pharmaceutically acceptable salt or hydrate thereof is administered daily.

In some embodiments, the fedratinib or pharmaceutically acceptable salt or hydrate thereof is administered orally.

In some embodiments, the fedratinib or pharmaceutically acceptable salt or hydrate thereof is administered at a dosage of 400 mg/day.

In some embodiments, the ActRIIB ligand trap is administered once at the beginning of every treatment cycle, wherein each cycle is 21 days.

In some embodiments, the ActRIIB ligand trap is administered to the subject for at least 1, 2, 3, 4, 5, 6, 7, 8, 9, or 10 cycles.

In some embodiments, the ActRIIB ligand trap is administered to the subject subcutaneously.

In some embodiments, the pharmaceutically effective amount of the ActRIIB ligand trap administered is 0.6 mg/kg, 0.8 mg/kg, 1 mg/kg, 1.33 mg/kg, or 1.75 mg/kg.

In some embodiments, the pharmaceutically effective amount of the ActRIIB ligand trap administered is 1.33 mg/kg.

In some embodiments, the first measurement of hemoglobin (Hgb) level is taken prior to the administration of the initial dose of the ActRIIB ligand trap.

In some embodiments, the first measurement of hemoglobin (Hgb) level is taken concurrently with the administration of the initial dose of the ActRIIB ligand trap, or taken about 3 weeks, 6 weeks, 9 weeks, 12 weeks, 15 weeks, 18 weeks, 21 weeks, or 24 weeks after the administration of the initial dose of the ActRIIB ligand trap.

In some embodiments, the second measurement of hemoglobin (Hgb) level is taken about 3 weeks, 6 weeks, 9 weeks, 12 weeks, 15 weeks, 18 weeks, 21 weeks, 24 weeks, 7 months, 8 months, 9 months, 10 months, 11 months, or 12 months after the initial dose of the ActRIIB ligand trap is administered to the subject.

In some embodiments, the first period of time is 1 day, 2 days, 3 days, 4 days, 5 days, 6 days, 1 week, 2 weeks, 3 weeks, 4 weeks, 5 weeks, 6 weeks, 7 weeks, or 8 weeks.

In some embodiments, the first period of time is 6 weeks.

In some embodiments, the initial dose of the ActRIIB ligand trap is 1.33 mg/kg.

In some embodiments, the subsequent dose of the ActRIIB ligand trap is 0.6 mg/kg, 0.8 mg/kg, 1.0 mg/kg, 1.33 mg/kg, or 1.75 mg/kg.

In some embodiments, the subsequent dose of the ActRIIB ligand trap is 1.33 mg/kg.

In some embodiments, when the second measurement of Hgb level is 2 g/dL or more higher than the first measurement of Hgb level, the subsequent dose of the ActRIIB ligand trap is lower than the initial dose of the ActRIIB ligand trap.

In some embodiments, when the subject has one or more RBC transfusion during the first period of time, or the second measurement of Hgb level is between 0 to about 1 g/dL higher than the first measurement of Hgb level, or the first measurement of Hgb level decreases 1 g/dL or more in a transfusion-free period of approximately 6 weeks, the subsequent dose of the ActRIIB ligand trap is higher than the initial dose of the ActRIIB ligand trap.

In some embodiments, the subsequent dose of the ActRIIB ligand trap is the same as the initial dose of the ActRIIB ligand trap.

In some embodiments, the methods further comprising: grading hematological, hepatic, non-hematological, or gastrointestinal event in the subject as Grade 1, 2, 3, 4, or 5 according to the National Cancer Institute (NCI) Common Terminology Criteria for Adverse Events (CTCAE); and administering a subsequent dose of fedratinib or pharmaceutically acceptable salt or hydrate thereof.

In some embodiments, the subsequent dose of fedratinib or pharmaceutically acceptable salt or hydrate thereof is 300 mg/day, 200 mg/day, or 100 mg/day.

In some embodiments, the ActRIIB ligand trap is a humanized fusion-protein consisting of the extracellular domain of ActRIIB and the human IgG1 Fc domain.

In some embodiments, the ActRIIB ligand trap is a fusion-protein comprising the extracellular domain of ActRIIB and the human IgG1 Fc domain.

In some embodiments, the ActRIIB ligand trap is a polypeptide comprising an amino acid sequence selected from the group consisting of: 90% identical to SEQ ID NO:3; 95% identical to SEQ ID NO:3; 98% identical to SEQ ID NO:3; SEQ ID NO:3; 90% identical to SEQ ID NO:6; 95% identical to SEQ ID NO:6; 98% identical to SEQ ID NO:6; SEQ ID NO:6; 90% identical to SEQ ID NO:7; 95% identical to SEQ ID NO:7; 98% identical to SEQ ID NO:7; SEQ ID NO:7; 90% identical to SEQ ID NO:11; 95% identical to SEQ ID NO:11; 98% identical to SEQ ID NO: 11; and SEQ ID NO: 11.

In some embodiments, the ActRIIB ligand trap is a polypeptide comprising an amino acid sequence selected from the group consisting of: 90% identical to SEQ ID NO: 11; 95% identical to SEQ ID NO: 11; 98% identical to SEQ ID NO: 11; and SEQ ID NO:11.

In some embodiments, the ActRIIB ligand trap is a polypeptide comprising the amino acid sequence of SEQ ID NO:11.

In some embodiments, the ActRIIB ligand trap is a polypeptide is encoded by the nucleotide sequence of SEQ ID NO:34 or a degenerate version of SEQ ID NO:34.

In some embodiments, the method increases the hemoglobin (Hgb) level in the subject by at least 0.5 g/dL, at least 1.0 g/dL, at least 1.5 g/dL, at least 2.0 g/dL, or at least 2.5 g/dL.

In some embodiments, the method increases the hemoglobin (Hgb) level in the subject by at least 1.5 g/dL.

In some embodiments, the method reduces at least 2, at least 3, at least 4, at least 5, at least 6, at least 7, at least 8, at least 9, or at least 10 units of red blood cell (RBC) transfusions received by the subject within a period of 56 days.

In some embodiments, the method reduces at least 4 units of RBC transfusions received by the subject within a period of 56 days.

In some embodiments, the method reduces at least 2, at least 3, at least 4, at least 5, at least 6, at least 7, at least 8, at least 9, or at least 10 units of red blood cell (RBC) transfusions received by the subject within a period of 84 days.

In some embodiments, the method increases the hemoglobin (Hgb) level in the subject by at least 1.5 g/dL over a consecutive 84-day period.

In some embodiments, the subject becomes red blood cell (RBC) transfusion free over a consecutive period of 84 days.

In some embodiments, the method increases hemoglobin (HGB) levels in the subject to levels equal to or about 1%, 2%, 3%, 4%, 5%, 6%, 7%, 8%, 9%, 10%, 20%, 25%, 30%, 40%, 50%, 60%, 70%, 75%, 80%, 90%, 100%, 200%, or 500% greater than HGB levels in the subject prior to said treating.

In some embodiments, the method increases hematocrit (HCT) levels in the subject to levels equal to or about 1%, 2%, 3%, 4%, 5%, 6%, 7%, 8%, 9%, 10%, 20%, 25%, 30%, 40%, 50%, 60%, 70%, 75%, 80%, 90%, 100%, 200%, or 500% greater than HCT levels in the subject prior to said treating.

In some embodiments, the method reduces mean corpuscular volume (MCV) levels in the subject to levels equal to or about 1%, 2%, 3%, 4%, 5%, 6%, 7%, 8%, 9%, 10%, 20%, 25%, 30%, 40%, 50%, 60%, 70%, 75%, 80%, 90%, or 100%, less than MCV levels in the subject prior to said treating.

In some embodiments, the method increases cellular hemoglobin concentration (CHC) levels in the subject to levels equal to or about 1%, 2%, 3%, 4%, 5%, 6%, 7%, 8%, 9%, 10%, 20%, 25%, 30%, 40%, 50%, 60%, 70%, 75%, 80%, 90%, 100%, 200%, or 500% greater than CHC levels in the subject prior to said treating

In some embodiments, the method reduces red blood cell distribution width (RDW) levels in the subject to levels equal to or about 1%, 2%, 3%, 4%, 5%, 6%, 7%, 8%, 9%, 10%, 20%, 25%, 30%, 40%, 50%, 60%, 70%, 75%, 80%, 90%, or 100%, less than the RDW levels in the subject prior to said treating

In some embodiments, the levels of reticulocytes in the subject remain in the range equal to or about 0.1%, 0.5%, 1%, 2%, 3%, 4%, 5%, 6%, 7%, 8%, 9%, 10%, 15%, or 20% above or below the levels of reticulocytes in the subject prior to said treating.

In some embodiments, the levels of reticulocytes in the subject remain in the range equal to or about 0.1%, 0.5%, 1%, 2%, 3%, 4%, 5%, 6%, 7%, 8%, 9%, 10%, 15%, or 20% above or below the levels of reticulocytes in a reference population.

In some embodiments, the levels of white blood cells in the subject remain in the range equal to or about 0.1%, 0.5%, 1%, 2%, 3%, 4%, 5%, 6%, 7%, 8%, 9%, 10%, 15%, or 20% above or below the levels of white blood cells in the subject prior to said treating.

### 4. BRIEF DESCRIPTION OF THE DRAWINGS

FIG. 1 illustrates the overall study design of treatment of patient with combination of luspatercept and fedratinib.

### 5. DETAILED DESCRIPTION

### 5.1 Abbreviations and Terminology

As used herein, the term “about” when used in conjunction with a number refers to any number within 1%, 2%, 3%, 4%, 5%, 6%, 7%, 8%, 9%, 10%, 11%, 12%, 13%, 14%, or 15% of the referenced number. In certain embodiments, the term “about” encompasses the exact number recited.

As used herein, “ActRII” refers to activin receptor type II. As used herein, “ActRIIB” refers to activin receptor type IIB. See, for example, Attisano et al., 1992, Cell 68: 97-108. GenBank™ accession number NM_001106.3 provides an exemplary human ActRIIB nucleic acid sequence. GenBank™ accession number NP_001097.2 provides an exemplary human ActRIIB amino acid sequence.

“β⁰” refers to an allele associated with a lack of beta globin subunit synthesis.

“β⁺” refers to an allele associated with reduced beta globin subunit synthesis.

As used herein, “BL” refers to baseline.

As used herein, “CHC” refers to cellular hemoglobin concentration.

As used herein, “ECD” refers to extracellular domain.

As used herein, “EPO” refers to erythropoietin.

As used herein, “fedratinib” refers to the compound of N-tert-butyl-3-[(5-methyl-2-{4-[2-(pyrrolidin-1-yl)ethoxy]anilino}pyrimidin-4-yl)amino]benzenesulfonamide (CAS No. 936091-26-8) and pharmaceutically acceptable salts and/or hydrates thereof (such as dichloride monohydrate). It is also known as SAR302503 or TG101348, and has the structure:

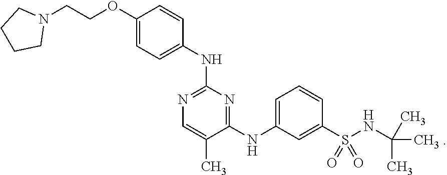 [SMILES: Cc1cnc(Nc2ccc(OCCN3CCCC3)cc2)nc1Nc1cccc(S(=O)(=O)NC(C)(C)C)c1]

As used herein, “Hb” and “Hgb” both refer to hemoglobin.

As used herein, “HI-E” refers to erythroid hematological improvement. In certain embodiments, the HI-E is as defined by IWG. In certain embodiments, the HI-E is as defined by the modified 2006 IWG. In certain embodiments, the HI-E for a low transfusion burden patient is an increase in hemoglobin concentration in the patient of at least 1.5 g/dL for at least 8 weeks. In certain embodiments, the HI-E for a high transfusion burden patient is an at least 4 unit reduction in RBC transfusion over 8 weeks.

As used herein, “HTB” refers to high transfusion burden. In certain embodiments, a HTB subject receives greater than or equal to 4 RBC units over the course of 8 weeks.

As used herein, “IgG” refers to immunoglobulin G.

As used herein, “IPSS-R” refers to International Prognostic Scoring System—Revised.

As used herein, “IWG” refers to International Working Group. See, e.g., Cheson et al. Blood. 2000 96:3671-3674. In certain embodiments, IWG refers to the modified 2006 criteria. See, e.g., Cheson et al., 2006, Blood, 108(2).

As used herein, “LTB” refers to low transfusion burden. In certain embodiments, a LTB subject receives less than 4 RBC units over the course of 8 weeks.

As used herein, “luspatercept” refers to a product resulting from expression from an opening reading frame with the nucleotide sequence of SEQ ID NO:34 or a degenerate version of SEQ ID NO:34 that encodes SEQ ID NO:11, and subsequent protein purification procedures.

As used herein, “MedDRA” refers to Medical Dictionary for Regulatory Activities.

As used herein, “MCV” refers to mean corpuscular volume.

As used herein, “MDS” refers to myelodysplastic syndromes.

As used herein, “mg/kg”, in the context of a dose of an ActRIIB ligand trap, refers to milligrams of the ActRIIB ligand trap per kilogram of the weight of the subject to whom the ActRIIB ligand trap is to be administered.

As used herein, “MPN-associated myelofibrosis” refers to myeloproliferative neoplasm-associated myelofibrosis.

As used herein, “PD” refers to pharmacodynamic.

As used herein, “PK” refers to pharmacokinetic.

As used herein, “PMF” refers to Primary Myelofibrosis.

As used herein, “post-ET MF” refers to Post-Essential Thrombocythemia Myelofibrosis.

As used herein, “post-PV MF” refers to Post-Polycythemia Vera Myelofibrosis.

As used herein, “RA” refers to refractory anemia.

As used herein, “RAEB” refers to refractory anemia with an excess of blasts.

As used herein, “RBC” refers to red blood cells.

As used herein, “RBC-TI” refers to red blood cell transfusion independent.

As used herein, “RDW” refers to red blood cell distribution width.

As used herein, “SC” refers to subcutaneous.

As used herein, “WPSS” refers to World Health Organization (WHO) Prognostic Scoring System.

In certain embodiments, “treat,” “treatment,” or “treating,” in the context of anemia, includes amelioration of at least one symptom of anemia. Non-limiting examples of anemia include fatigue, loss of energy, rapid heartbeat, shortness of breath, headaches, difficulty concentrating, dizziness, pale skin, leg cramps, and insomnia.

### 5.2 Overview

Provided herein are methods of treating anemia in a subject in need thereof comprising: administering to the subject an ActRIIB ligand inhibitor (e.g., Luspatercept, a product resulting from expression from an opening reading frame with the nucleotide sequence of SEQ ID NO:34 or a degenerate version of SEQ ID NO:34 that encodes SEQ ID NO:11, and subsequent protein purification procedures; see Section 5.4), and administering to the subject fedratinib or pharmaceutically acceptable salt and/or hydrate thereof (see Section 5.3). Dosing regimens are described in Sections 5.5 and 5.8. Patient populations that can be treated using the methods provided herein are described in Section 5.6.

### 5.3 Fedratinib

Fedratinib (INREBIC®) is an oral kinase inhibitor with activity against wild type and mutationally activated Janus Kinase 2 (JAK2) and fibromyalgia syndrome (FMS)-like tyrosine kinase 3 (FLT3). Fedratinib is a JAK2-selective inhibitor with higher potency for JAK2 over family members JAK1, JAK3 and tyrosine kinase 2 (TYK2). In cell models expressing mutationally active JAK2 or FLT3, fedratinib reduced phosphorylation of signal transducer and activator of transcription (STAT3/5) proteins, inhibited cell proliferation, and induced apoptotic cell death. In mouse models of JAK2V617F-driven myeloproliferative disease, fedratinib blocked phosphorylation of STAT3/5, increased survival and improved disease-associated symptoms, including reduction of white blood cells, hematocrit, splenomegaly, and fibrosis.

Fedratinib demonstrated clinical efficacy in a randomized, placebo-controlled, Phase 3 study (JAKARTA [EFC12153]) in patients with intermediate-2 or high-risk MF who were previously untreated.

On Aug. 16, 2019, fedratinib (INREBIC®) was approved by the US FDA for the treatment of adult patients with intermediate-2 or high-risk primary or secondary (post-polycythemia vera or post-essential thrombocythemia) myelofibrosis.

In certain embodiments, fedratinib or a pharmaceutically acceptable salt and/or hydrate thereof can be used in the methods provided herein (see, Section 5.5).

In some embodiments, as used herein, “fedratinib” refers to fedratinib or a pharmaceutically acceptable salt and/or hydrate thereof. In some embodiments, “fedratinib” refers to fedratinib dihydrochloride. In some embodiments, “fedratinib” refers to fedratinib dihydrochloride monohydrate.

### 5.4 ActRIIB Ligand Trap

In certain embodiments, the ActRIIB ligand traps described in this Section can be used in the methods provided herein (see, Section 5.5). In certain embodiments, the ActRIIB ligand trap for use with the present methods comprises an amino acid sequence of SEQ ID NO:11. In certain embodiments, the ActRIIB ligand trap for use with the present methods is a product resulting from expression from an opening reading frame with the nucleotide sequence of SEQ ID NO:34 or a degenerate version of SEQ ID NO:34 that encodes SEQ ID NO:11.

As used herein, the term “ActRIIB” refers to a family of activin receptor type IIB (ActRIIB) proteins from any species and variants derived from such ActRIIB proteins by mutagenesis or other modification. Reference to ActRIIB herein is understood to be a reference to any one of the currently identified forms of the receptor. Members of the ActRIIB family are generally transmembrane proteins, composed of a ligand-binding extracellular domain with a cysteine-rich region, a transmembrane domain, and a cytoplasmic domain with predicted serine/threonine kinase activity.

ActRIIB ligand traps to be used in the compositions and methods described herein include, without limitation, activin-binding soluble ActRIIB polypeptides; antibodies that bind to activin (particularly the activin A or B subunits, also referred to as betaA or betaB) and disrupt ActRIIB binding; antibodies that bind to ActRIIB and disrupt activin binding; non-antibody proteins selected for activin or ActRIIB binding; and randomized peptides selected for activin or ActRIIB binding, which can be conjugated to an Fc domain.

In certain embodiments, two or more different proteins (or other moieties) with activin or ActRIIB binding activity, especially activin binders that block the type I (e.g., a soluble type I activin receptor) and type II (e.g., a soluble type II activin receptor) binding sites, respectively, may be linked together to create a bifunctional or multifunctional binding molecule that inhibits ActRIIB and thus can be used in the compositions and methods described herein include. In certain embodiments, Activin-ActRIIB signaling axis antagonists that inhibit ActRIIB include nucleic acid aptamers, small molecules and other agents are used in the compositions and methods described herein include.

Such ActRIIB ligand traps can be generated and modified as previously described in Section 5.5.2 of International Publication No. WO 2014/066486, which is incorporated herein in its entirety.

(a) ActRIIB Ligand Traps Comprising ActRIIB Antibodies

In certain embodiments, the ActRIIB ligand traps to be used in the compositions and methods described herein include antibodies that bind to activin (particularly the activin A or B subunits) and disrupt ActRIIB binding.

(b) ActRIIB Ligand Traps Comprising ActRIIB Polypeptides

As used herein, the term “ActRIIB polypeptide” refers to polypeptides comprising any naturally occurring polypeptide of an ActRIIB family member as well as any variants thereof (including mutants, fragments, fusions, and peptidomimetic forms) that retain a useful activity. For example, ActRIIB polypeptides include polypeptides derived from the sequence of any known ActRIIB receptor having a sequence at least about 80% identical to the sequence of an ActRIIB polypeptide, and optionally at least 85%, 90%, 95%, 96%, 97%, 98%, 99% or greater identity. For example, an ActRIIB polypeptide may bind to and inhibit the function of an ActRIIB protein and/or activin. An example of an ActRIIB polypeptide includes the human ActRIIB precursor polypeptide (SEQ ID NO:2 or SEQ ID NO:14). With respect to the ActRIIB precursor polypeptide whose amino acid sequence is depicted as SEQ ID NO:2 or SEQ ID NO:14 (i.e., the human ActRIIB precursor polypeptide), the signal peptide of the ActRIIB precursor polypeptide is located at amino acids 1 to 18; the extracellular domain is located at amino acids 19 to 134 and the potential N-linked glycosylation sites are located at amino acid positions 42 and 65. The nucleic acid sequence encoding the human ActRIIB precursor polypeptide of SEQ ID NO:2 is disclosed as SEQ ID NO:5 (SEQ ID NO:5 provides an alanine at the codon corresponding to amino acid position 64, but could be readily modified by one of skill in the art using methods known in the art to provide an arginine at the codon corresponding to amino acid position 64 instead). See Table 1 for a description of the sequences.

The numbering of amino acids for all of the ActRIIB-related polypeptides described herein is based on the amino acid numbering for SEQ ID NO:2 and SEQ ID NO:14 (which only differ in the amino acid expressed at position 64), unless specifically designated otherwise. For example, if an ActRIIB polypeptide is described as having a substitution/mutation at amino acid position 79, then it is to be understood that position 79 refers to the 79th amino acid in SEQ ID NO:2 or SEQ ID NO:14, from which the ActRIIB polypeptide is derived. Likewise, if an ActRIIB polypeptide is described as having an alanine or an arginine at amino acid position 64, then it is to be understood that position 64 refers to the 64th amino acid in SEQ ID NO:2 or SEQ ID NO:14, from which the ActRIIB polypeptide is derived.

In certain embodiments, the ActRIIB ligand traps used in the compositions and methods described herein comprise polypeptides comprising an activin-binding domain of ActRIIB. In certain embodiments, the activin-binding domains of ActRIIB comprise the extracellular domain of ActRIIB, or a portion thereof. In specific embodiments, the extracellular domain or portion thereof of ActRIIB is soluble. Illustrative modified forms of ActRIIB polypeptides are disclosed in U.S. Patent Application Publication Nos. 20090005308 and 20100068215, the disclosures of which are incorporated herein by reference in their entireties.

In specific embodiments, the ActRIIB ligand traps used in the compositions and methods described herein are soluble ActRIIB polypeptides. The term “soluble ActRIIB polypeptide” generally refers to polypeptides comprising an extracellular domain of an ActRIIB protein, including any naturally occurring extracellular domain of an ActRIIB protein as well as any variants thereof (including mutants, fragments and peptidomimetic forms). Soluble ActRIIB polypeptides can bind to activin; however, the wild type ActRIIB protein does not exhibit significant selectivity in binding to activin versus GDF8/11. In certain embodiments, altered forms of ActRIIB with different binding properties can be used in the methods provided herein. Such altered forms are disclosed, e.g., in international patent application publication Nos. WO 2006/012627 and WO 2010/019261, the disclosures of which are incorporated herein by reference in their entireties. Native or altered ActRIIB proteins may be given added specificity for activin by coupling them with a second, activin-selective binding agent. Exemplary soluble ActRIIB polypeptides include the extracellular domain of a human ActRIIB polypeptide (e.g., SEQ ID NOs: 3, 4, 9, 12, 13, 15, 16, 17, 18, 19, 22, 23, 24, and 29).

An Fc fusion protein having the ActRIIB extracellular sequence disclosed by Hilden et al. (Blood, 1994, 83(8):2163-70), which has an alanine at the position corresponding to amino acid 64 of the ActRIIB precursor amino acid sequence, i.e., SEQ ID NO: 2 (herein referred to as “A64”), has been demonstrated to possess a relatively low affinity for activin and GDF-11. By contrast, an Fc fusion protein with an arginine at position 64 of the ActRIIB precursor amino acid sequence (herein referred to as “R64”) has an affinity for activin and GDF-11 in the low nanomolar to high picomolar range (see, e.g., U.S. Patent Application Publication No. 20100068215, the disclosure of which is herein incorporated in its entirety). An ActRIIB precursor amino acid sequence with an arginine at position 64 is presented in SEQ ID NO:14. As such, in certain embodiments, the ActRIIB polypeptides used in accordance with the compositions and methods described herein may comprise either (i) an alanine at the position corresponding to amino acid 64 of the ActRIIB precursor amino acid sequence, i.e., SEQ ID NO:2; or (ii) an arginine at position 64 of the ActRIIB precursor amino acid sequence, i.e., SEQ ID NO:14. In other embodiments, the ActRIIB polypeptides used in accordance with the compositions and methods described herein may comprise an amino acid that is not alanine or arginine at the position corresponding to amino acid 64 of the ActRIIB precursor amino acid sequence, i.e., SEQ ID NO:2 or SEQ ID NO:14.

It has been shown that a deletion of the proline knot at the C-terminus of the extracellular domain of ActRIIB reduces the affinity of the receptor for activin (see, e.g., Attisano et al., Cell, 1992, 68(1):97-108). An ActRIIB-Fc fusion protein containing amino acids 20-119 of SEQ ID NO: 14 (i.e., SEQ ID NO:18), “ActRIIB(20-119)-Fc” has reduced binding to GDF-11 and activin relative to an ActRIIB-Fc fusion protein containing amino acids 20-134 of SEQ ID NO: 14 (i.e., SEQ ID NO:17), “ActRIIB(20-134)-Fc”, which includes the proline knot region and the complete juxtamembrane domain. However, an ActRIIB-Fc fusion protein containing amino acids 20-129 of SEQ ID NO: 14, “ActRIIB(20-129)-Fc” retains similar but somewhat reduced activity relative to the non-truncated extracellular domain of ActRIIB, even though the proline knot region is disrupted. Thus, ActRIIB polypeptides comprising extracellular domains that stop at amino acid 134, 133, 132, 131, 130 and 129 of SEQ ID NO:14 (or SEQ ID NO:2) are all expected to be active, but constructs stopping at amino acid 134 or 133 may be most active. Similarly, mutations at any of residues 129-134 are not expected to alter ligand binding affinity by large margins, as indicated by the fact that mutations of P129 and P130 of SEQ ID NO: 14 do not substantially decrease ligand binding. Therefore, the ActRIIB polypeptides used in accordance with the methods and compositions described herein may end as early as amino acid 109 (i.e., the final cysteine) of SEQ ID NO:14 (or SEQ ID NO:2), however, forms ending at or between amino acid positions 109 and 119 of SEQ ID NO:14 (or SEQ ID NO:2) are expected to have reduced ligand binding ability.

Amino acid 29 of SEQ ID NO:2 and SEQ ID NO:14 represents the initial cysteine in the ActRIIB precursor sequence. It is expected that an ActRIIB polypeptide beginning at amino acid 29 of the N-terminus of SEQ ID NO:2 or SEQ ID NO:14, or before these amino acid positions, will retain ligand binding activity. An alanine to asparagine mutation at position 24 of SEQ ID NO:2 or SEQ ID NO:14 introduces an N-linked glycosylation sequence without substantially affecting ligand binding. This confirms that mutations in the region between the signal cleavage peptide and the cysteine cross-linked region, corresponding to amino acids 20-29 of SEQ ID NO:2 or SEQ ID NO:14, are well tolerated. In particular, ActRIIB polypeptides beginning at amino acid position 20, 21, 22, 23 and 24 of SEQ ID NO:2 or SEQ ID NO:14 will retain activity, and ActRIIB polypeptides beginning at amino acid positions 25, 26, 27, 28 and 29 of SEQ ID NO:2 or SEQ ID NO:14 are also expected to retain activity. An ActRIIB polypeptide beginning at amino acid position 22, 23, 24 or 25 of SEQ ID NO:2 or SEQ ID NO:14 will have the most activity.

Taken together, the active portions (i.e., ActRIIB polypeptides) of the ActRIIB precursor protein (i.e., SEQ ID NO:2 or SEQ ID NO:14) to be used in accordance with the methods and compositions described herein will generally comprise amino acids 29-109 of SEQ ID NO:2 or SEQ ID NO:14, and such ActRIIB polypeptides may, for example, begin at a residue corresponding to any one of amino acids 19-29 of SEQ ID NO:2 or SEQ ID NO:4 and end at a position corresponding to any one of amino acids 109-134 of SEQ ID NO:2 or SEQ ID NO:14. Specific examples of ActRIIB polypeptides encompassed herein include those that begin at an amino acid position from 19-29, 20-29 or 21-29 of SEQ ID NO:2 or SEQ ID NO:14 and end at an amino acid position from 119-134, 119-133 or 129-134, 129-133 of SEQ ID NO:2 or SEQ ID NO:14. Other specific examples of ActRIIB polypeptides encompassed herein include those that begin at an amino acid position from 20-24 (or 21-24, or 22-25) of SEQ ID NO:2 or SEQ ID NO:14 and end at an amino acid position from 109-134 (or 109-133), 119-134 (or 119-133) or 129-134 (or 129-133) of SEQ ID NO:2 or SEQ ID NO:14. Variant ActRIIB polypeptides falling within these ranges are also contemplated, particularly those having at least 80%, 85%, 90%, 91%, 92%, 93%, 94%, 95%, 96%, 97%, 98%, or 99% sequence identity or sequence homology to the corresponding portion of SEQ ID NO:2 or SEQ ID NO: 14.

In certain embodiments, the ActRIIB ligand traps used in the compositions and methods described herein comprise a truncated form of an extracellular domain of ActRIIB. The truncation can be at the carboxy terminus and/or the amino terminus of the ActRIIB polypeptide. In certain embodiments, the truncation can be 1, 2, 3, 4, 5, 6, 7, 8, 9, 10, 11, 12, 13, 14, 15, 16, 17, 18, 19, 20, 21, 22, 23, 24, or 25 amino acids long relative to the mature ActRIIB polypeptide extracellular domain. In certain embodiments, the truncation can be 1, 2, 3, 4, 5, 6, 7, 8, 9, 10, 11, 12, 13, 14, 15, 16, 17, 18, 19, 20, 21, 22, 23, 24, or 25 N-terminal amino acids of the mature ActRIIB polypeptide extracellular domain. In certain embodiments, the truncation can be 1, 2, 3, 4, 5, 6, 7, 8, 9, 10, 11, 12, 13, 14, 15, 16, 17, 18, 19, 20, 21, 22, 23, 24, or 25 C-terminal amino acids of the mature ActRIIB polypeptide extracellular domain. For example, truncated forms of ActRIIB include polypeptides with amino acids 20-119; 20-128; 20-129; 20-130; 20-131; 20-132; 20-133; 20-134; 20-131; 21-131; 22-131; 23-131; 24-131; and 25-131, wherein the amino acid positions refer to the amino acid positions in SEQ ID NO:2 or SEQ ID NO:14.

Additional exemplary truncated forms of ActRIIB include (i) polypeptides beginning at amino acids at any of amino acids 21-29 of SEQ ID NO:2 or SEQ ID NO:14 (optionally beginning at 22-25 of SEQ ID NO:2 or SEQ ID NO:14) and ending at any of amino acids 109-134 of SEQ ID NO:2 or SEQ ID NO:14; (ii) polypeptides beginning at any of amino acids 20-29 of SEQ ID NO:2 or SEQ ID NO:14 (optionally beginning at 22-25 of SEQ ID NO:2 or SEQ ID NO:14) and ending at any of amino acids 109-133 of SEQ ID NO:2 or SEQ ID NO:14; (iii) polypeptides beginning at any of amino acids 20-24 of SEQ ID NO:2 or SEQ ID NO: 14 (optionally beginning at 22-25 of SEQ ID NO:2 or SEQ ID NO:14) and ending at any of amino acids 109-133 of SEQ ID NO:2 or SEQ ID NO:14; (iv) polypeptides beginning at any of amino acids 21-24 of SEQ ID NO:2 or SEQ ID NO:14 and ending at any of amino acids 109-134 of SEQ ID NO:2 or SEQ ID NO:14; (v) polypeptides beginning at any of amino acids 20-24 of SEQ ID NO:2 or SEQ ID NO:14 and ending at any of amino acids 118-133 of SEQ ID NO:2 or SEQ ID NO:14; (vi) polypeptides beginning at any of amino acids 21-24 of SEQ ID NO:2 or SEQ ID NO:14 and ending at any of amino acids 118-134 of SEQ ID NO:2 or SEQ ID NO:14; (vii) polypeptides beginning at any of amino acids 20-24 of SEQ ID NO:2 or SEQ ID NO:14 and ending at any of amino acids 128-133 of SEQ ID NO:2 or SEQ ID NO:14; (viii) polypeptides beginning at any of amino acids 20-24 of SEQ ID NO:2 or SEQ ID NO:14 and ending at any of amino acids 128-133 of SEQ ID NO:2 or SEQ ID NO:14; (ix) polypeptides beginning at any of amino acids 21-29 of SEQ ID NO:2 or SEQ ID NO:14 and ending at any of amino acids 118-134 of SEQ ID NO:2 or SEQ ID NO:14; (x) polypeptides beginning at any of amino acids 20-29 of SEQ ID NO:2 or SEQ ID NO:14 and ending at any of amino acids 118-133 of SEQ ID NO:2 or SEQ ID NO:14; (xi) polypeptides beginning at any of amino acids 21-29 of SEQ ID NO:2 or SEQ ID NO:4 and ending at any of amino acids 128-134 of SEQ ID NO:2 or SEQ ID NO:14; and (xii) polypeptides beginning at any of amino acids 20-29 of SEQ ID NO:2 or SEQ ID NO:14 and ending at any of amino acids 128-133 of SEQ ID NO:2 or SEQ ID NO:14. In a specific embodiment, an ActRIIB polypeptides comprises, consists essentially of, or consists of, an amino acid sequence beginning at amino acid position 25 of SEQ ID NO:2 or SEQ ID NO:4 and ending at amino acid position 131 of SEQ ID NO:2 or SEQ ID NO:14. In another specific embodiment, an ActRIIB polypeptide consists of, or consists essentially of, the amino acid sequence of SEQ ID NO:3, 4, 9, 12, 13, 15, 16, 17, 18, 19, 22, 23, 28, or 29.

Any of the ActRIIB polypeptides used in the compositions and methods described herein may be produced as a homodimer. Any of the ActRIIB polypeptides used in the compositions and methods described herein may be formulated as a fusion protein having a heterologous portion that comprises a constant region from an IgG heavy chain, such as an Fc domain. Any of the ActRIIB polypeptides used in the compositions and methods described herein may comprise an acidic amino acid at the position corresponding to position 79 of SEQ ID NO:2 or SEQ ID NO:14, optionally in combination with one or more additional amino acid substitutions, deletions or insertions relative to SEQ ID NO:2 or SEQ ID NO:14.

In specific embodiments, the ActRIIB ligand traps used in the compositions and methods described herein comprise an extracellular domain of ActRIIB with one or more amino acid substitutions/mutations. Such an amino acid substitution/mutation can be, for example, an exchange from the leucine at amino acid position 79 of SEQ ID NO:2 or SEQ ID NO:14 to an acidic amino acid, such as aspartic acid or glutamic acid. For example, position L79 of SEQ ID NO:2 or SEQ ID NO:14 may be altered in ActRIIB extracellular domain polypeptides to confer altered activin-myostatin (GDF-11) binding properties. L79A and L79P mutations reduce GDF-11 binding to a greater extent than activin binding. L79E and L79D mutations retain GDF-11 binding, while demonstrating greatly reduced activin binding.

In certain embodiments, the ActRIIB ligand traps used in the compositions and methods described herein comprise a truncated form of an ActRIIB extracellular domain that also carries an amino acid substitution, e.g., an exchange from the leucine at amino acid position 79 of SEQ ID NO:2 or SEQ ID NO:14 to an acidic amino acid, such as aspartic acid or glutamic acid. In a specific embodiment, the truncated form of an extracellular domain of ActRIIB polypeptide that also carries an amino acid substitution used in the compositions and methods described herein is SEQ ID NO:9. Forms of ActRIIB that are truncated and/or carry one or more amino acid substitutions can be linked to an Fc domain of an antibody as discussed above.

Functionally active fragments of ActRIIB polypeptides can be obtained, for example, by screening polypeptides recombinantly produced from the corresponding fragment of the nucleic acid encoding an ActRIIB polypeptide. In addition, fragments can be chemically synthesized using techniques known in the art such as conventional Merrifield solid phase f-Moc or t-Boc chemistry. The fragments can be produced (recombinantly or by chemical synthesis) and tested to identify those peptidyl fragments that can function as antagonists (traps) of ActRIIB protein or signaling mediated by activin.

In addition, functionally active variants of ActRIIB polypeptides can be obtained, for example, by screening libraries of modified polypeptides recombinantly produced from the corresponding mutagenized nucleic acids encoding an ActRIIB polypeptide. The variants can be produced and tested to identify those that can function as antagonists (inhibitors) of ActRIIB protein or signaling mediated by activin. In certain embodiments, a functional variant of the ActRIIB polypeptides comprises an amino acid sequence that is at least 75% identical to an amino acid sequence selected from SEQ ID NO:3, 4, 9, 12, 13, 15, 16, 17, 18, 19, 22, 23, 28, and 29. In certain embodiments, the functional variant has an amino acid sequence at least 80%, 85%, 90%, 95%, 96%, 97%, 98%, or 99% identical to an amino acid sequence selected from SEQ ID NO: 3, 4, 9, 12, 13, 15, 16, 17, 18, 19, 22, 23, 28, and 29.

It has been demonstrated that the ligand binding pocket of ActRIIB is defined by residues Y31, N33, N35, L38 through T41, E47, E50, Q53 through K55, L57, H58, Y60, S62, K74, W78 through N83, Y85, R87, A92, and E94 through F101 of SEQ ID NO:2 or SEQ ID NO:14. At these positions, it is expected that conservative mutations will be tolerated, although a K74A mutation is well-tolerated, as are R40A, K55A, F82A and mutations at position L79. R40 is a K in Xenopus, indicating that basic amino acids at this position will be tolerated. Q53 is R in bovine ActRIIB and K in Xenopus ActRIIB, and therefore amino acids including R, K, Q, N and H will be tolerated at this position. Thus, a general formula for an ActRIIB polypeptide for use in the methods and compositions described herein is one that comprises amino acids 29-109 of SEQ ID NO:2 or SEQ ID NO:14, but optionally beginning at an amino acid position ranging from 20-24 or 22-25 of SEQ ID NO:2 or SEQ ID NO:14 and ending at an amino acid position ranging from 129-134 of SEQ ID NO:2 or SEQ ID NO:14, and comprising no more than 1, 2, 5, or 15 conservative amino acid changes in the ligand binding pocket, and zero, one or more non-conservative alterations at amino acid positions 40, 53, 55, 74, 79 and/or 82 of SEQ ID NO:2 or SEQ ID NO:14 in the ligand binding pocket. Such an ActRIIB polypeptide may retain greater than 80%, 90%, 95% or 99% sequence identity or sequence homology to the sequence of amino acids 29-109 of SEQ ID NO:2 or SEQ ID NO:14. Sites outside the binding pocket, at which variability may be particularly well tolerated, include the amino and carboxy termini of the extracellular domain of ActRIIB, and positions 42-46 and 65-73. An asparagine to alanine alteration at position 65 of SEQ ID NO:2 or SEQ ID NO:14 (N65A) actually improves ligand binding in the A64 background, and is thus expected to have no detrimental effect on ligand binding in the R64 background. This change probably eliminates glycosylation at N65 in the A64 background, thus demonstrating that a significant change in this region is likely to be tolerated. While an R64A change is poorly tolerated, R64K is well-tolerated, and thus another basic residue, such as H may be tolerated at position 64.

In specific embodiments, the ActRIIB ligand traps used in the compositions and methods described herein comprise a conjugate/fusion protein comprising an extracellular domain (e.g., an activin-binding domain) of an ActRIIB receptor linked to an Fc portion of an antibody. Such conjugate/fusion proteins may comprise any of the ActRIIB polypeptides disclosed herein (e.g., any of SEQ ID NOs: 3, 4, 9, 12, 13, 15, 16, 17, 18, 19, 22, 23, 28, and 29), any ActRIIB polypeptides known in the art, or any ActRIIB polypeptides generated using methods known in the art and/or provided herein.

In certain embodiments, the extracellular domain is linked to an Fc portion of an antibody via a linker, e.g., a peptide linker. Exemplary linkers include short polypeptide sequences such as 2-10, 2-5, 2-4, 2-3 amino acid residues (e.g., glycine residues), such as, for example, a Gly-Gly-Gly linker. In a specific embodiment, the linker comprises the amino acid sequence Gly-Gly-Gly (GGG). In another specific embodiment, the linker comprises the amino acid sequence Thr-Gly-Gly-Gly (TGGG). Optionally, the Fc domain has one or more mutations at residues such as Asp-265, lysine 322, and Asn-434. In certain cases, the mutant Fc domain having one or more of these mutations (e.g., an Asp-265 mutation) has a reduced ability to bind to the Fcγ receptor relative to a wild-type Fc domain. In other cases, the mutant Fc domain having one or more of these mutations (e.g., an Asn-434 mutation) has an increased ability to bind to the MHC class I-related Fc-receptor (FcRN) relative to a wild-type Fc domain. Exemplary fusion proteins comprising a soluble extracellular domain of ActRIIB fused to an Fc domain are set forth in SEQ ID NOs:6, 7, 10, 11, 20, 21, 24, 25, 26, 27, 30, 32, and 33.

In a specific embodiment, the ActRIIB ligand traps used in the compositions and methods described herein comprise the extracellular domain of ActRIIB, or a portion thereof, linked to an Fc portion of an antibody, wherein said ActRIIB ligand trap comprises an amino acid sequence that is at least 75% identical to an amino acid sequence selected from SEQ ID NOs: 6, 7, 10, 11, 20, 21, 24, 25, 26, 27, 30, 32, and 33. In another specific embodiment, the ActRIIB ligand traps used in the compositions and methods described herein comprise the extracellular domain of ActRIIB, or a portion thereof, linked to an Fc portion of an antibody, wherein said ActRIIB ligand trap comprises an amino acid sequence that is at least 80%, 85%, 90%, 95%, 96%, 97%, 98%, or 99% identical to an amino acid sequence selected from SEQ ID NOs: 6, 7, 10, 11, 20, 21, 24, 25, 26, 27, 30, 32, and 33.

In a specific embodiment, the ActRIIB ligand trap to be used in the compositions and methods described herein is a fusion protein between the extracellular domain of the human ActRIIB receptor and the Fc portion of IgG1. In another specific embodiment, the ActRIIB ligand trap to be used in the compositions and methods described herein is a fusion protein between a truncated extracellular domain of the human ActRIIB receptor and the Fc portion of IgG1. In another specific embodiment, the ActRIIB ligand trap to be used in the compositions and methods described herein is a fusion protein between a truncated extracellular domain of the human ActRIIB receptor and the Fc portion of IgG1, wherein the truncated extracellular domain of the human ActRIIB receptor possesses an amino acid substitution at the amino acid position corresponding to amino acid 79 of SEQ ID NO:2 or SEQ ID NO:14. In one embodiment, the amino acid substitution at the amino acid position corresponding to amino acid 79 of SEQ ID NO:2 or SEQ ID NO:14 is substitution of Leucine for Aspartic Acid (i.e., an L79D mutation).

In a specific embodiment, the ActRIIB ligand trap to be used in the compositions and methods described herein is SEQ ID NO:10 or 11, which represents a fusion protein between the extracellular domain of the human ActRIIB receptor and the Fc portion of IgG1, wherein said ActRIIB extracellular domain comprises amino acids 25-131 of SEQ ID NO:14 with an L79D mutation. The nucleic acid sequence encoding the ActRIIB-Fc fusion protein of SEQ ID NO:10 is presented in SEQ ID NO:31.

In another specific embodiment, the ActRIIB ligand trap to be used in the compositions and methods described herein is SEQ ID NO:20 or 21, which represents a fusion protein between the extracellular domain of the human ActRIIB receptor and the Fc portion of IgG1, wherein said ActRIIB extracellular domain comprises amino acids 25-131 of SEQ ID NO:2 with an L79D mutation.

In specific embodiments, mutated ActRIIB polypeptides comprising the addition of a further N-linked glycosylation site (N-X-S/T) that increases the serum half-life of an ActRIIB-Fc fusion protein, relative to the ActRIIB(R64)-Fc form can be used in the methods and compositions described herein. In a specific embodiment, introduction of an asparagine at position 24 of SEQ ID NO:2 or SEQ ID NO:14 (A24N) results in the creation of an NXT sequence that confers a longer half-life. Other NX(T/S) sequences can be found at 42-44 (NQS) and 65-67 (NSS), although the latter may not be efficiently glycosylated with the R at position 64 (i.e., in R64 polypeptides). N-X-S/T sequences may be generally introduced at positions outside the ligand binding pocket of ActRIIB, which is detailed above. Particularly suitable sites for the introduction of non-endogenous N-X-S/T sequences include amino acids 20-29, 20-24, 22-25, 109-134, 120-134 or 129-134 of SEQ ID NO:2 or SEQ ID NO:14. N-X-S/T sequences may also be introduced into the linker between the ActRIIB sequence and the Fc or other fusion component. Such a site may be introduced with minimal effort by introducing an N in the correct position with respect to a pre-existing S or T, or by introducing an S or T at a position corresponding to a pre-existing N. Thus, desirable alterations that would create an N-linked glycosylation site are: A24N, R64N, S67N (possibly combined with an N65A alteration), E106N, R112N, G120N, E123N, P129N, A132N, R112S and R112T (with all amino acid positions corresponding to the positions they can be found in SEQ ID NO:2 or SEQ ID NO:14). Any S that is predicted to be glycosylated may be altered to a T without creating an immunogenic site, because of the protection afforded by the glycosylation. Likewise, any T that is predicted to be glycosylated may be altered to an S. Thus the alterations S67T and S44T are encompassed herein. Likewise, in an A24N variant, an S26T alteration may be used. Accordingly, an ActRIIB polypeptide may include one or more additional, non-endogenous N-linked glycosylation consensus sequences.

In certain embodiments, the methods and compositions described herein use isolated or purified ActRIIB polypeptides, i.e., ActRIIB polypeptides which are isolated from, or otherwise substantially free of, other proteins can be used with the methods and compositions described herein. ActRIIB polypeptides will generally be produced by expression from recombinant nucleic acids.

In certain aspects, the ActRIIB polypeptides used in the methods and compositions described herein are encoded by isolated and/or recombinant nucleic acids, including fragments, functional variants and fusion proteins disclosed herein. For example, SEQ ID NO:5 encodes the naturally occurring human ActRIIB precursor polypeptide. The subject nucleic acids may be single-stranded or double stranded. Such nucleic acids may be DNA or RNA molecules. These nucleic acids may be used, for example, in methods for making ActRIIB polypeptides or as direct therapeutic agents (e.g., in a gene therapy approach).

In certain aspects, the nucleic acids that can be used to produce ActRIIB polypeptides suitable for use in the methods and compositions described herein are further understood to include nucleic acids that are variants of SEQ ID NO:5 as well as variants of those nucleic acid sequences that encode soluble ActRIIB polypeptides (e.g., nucleic acids that encode SEQ ID NOs: 3, 4, 9, 12, 13, 15, 16, 17, 18, 19, 22, 23, 28, and 29). Variant nucleotide sequences include sequences that differ by one or more nucleotide substitutions, additions or deletions, such as allelic variants.

In certain embodiments, the isolated or recombinant nucleic acid sequences that can be used to produce ActRIIB polypeptides suitable for use in the methods and compositions described herein are at least 80%, 85%, 90%, 95%, 97%, 98%, 99% or 100% identical to SEQ ID NO:5 or those nucleic acid sequences that encode soluble ActRIIB polypeptides (e.g., nucleic acids that encode SEQ ID NOs: 3, 4, 9, 12, 13, 15, 16, 17, 18, 19, 22, 23, 28, and 29). One of ordinary skill in the art will appreciate that nucleic acid sequences complementary to SEQ ID NO:5 or those nucleic acid sequences that encode soluble ActRIIB polypeptides (e.g., nucleic acids that encode SEQ ID NOs: 3, 4, 9, 12, 13, 15, 16, 17, 18, 19, 22, 23, 28, and 29), and variants of SEQ ID NO:19 or those nucleic acid sequences that encode soluble ActRIIB polypeptides (e.g., nucleic acids that encode SEQ ID NOs: 3, 4, 9, 12, 13, 15, 16, 17, 18, 19, 22, 23, 28, and 29) can be used with the methods and compositions described herein. In further embodiments, the nucleic acid sequences can be isolated, recombinant, and/or fused with a heterologous nucleotide sequence, or in a DNA library.

### Assays

Various ActRIIB polypeptide variants, or soluble ActRIIB polypeptide variants, may be tested for their ability to inhibit ActRIIB signaling. In addition, compounds can be tested for their ability to inhibit ActRIIB signaling. Once activity of ActRIIB ligand traps is confirmed, these compounds can be used with the methods provided herein. The assays below are described for ActRIIA but can be performed analogously for ActRIIB.

For example, the effect of an ActRIIA polypeptide variant on the expression of genes involved in bone production or bone destruction may be assessed. This may, as needed, be performed in the presence of one or more recombinant ActRIIA ligand proteins (e.g., activin), and cells may be transfected so as to produce an ActRIIA polypeptide and/or variants thereof, and optionally, an ActRIIA ligand. Likewise, an ActRIIA polypeptide may be administered to a mouse or other animal, and one or more bone properties, such as density or volume may be assessed. The healing rate for bone fractures may also be evaluated. Dual-energy x-ray absorptiometry (DEXA) is a well-established, non-invasive, quantitative technique for assessing bone density in an animal. In humans central DEXA systems may be used to evaluate bone density in the spine and pelvis. These are the best predictors of overall bone density. Peripheral DEXA systems may be used to evaluate bone density in peripheral bones, including, for example, the bones of the hand, wrist, ankle and foot. Traditional x-ray imaging systems, including CAT scans, may be used to evaluate bone growth and fracture healing. In addition, bone density can be measured using quantitative computed tomography (qCT). The mechanical strength of bone may also be evaluated.

In certain aspects, provided herein is the use of ActRIIA polypeptides (e.g., soluble ActRIIA polypeptides) and activin polypeptides to identify compounds (agents) which are agonist or antagonists of the activin-ActRIIA signaling pathway. Compounds identified through this screening can be tested to assess their ability to modulate bone growth or mineralization in vitro. Optionally, these compounds can further be tested in animal models to assess their ability to modulate tissue growth in vivo.

There are numerous approaches to screening for therapeutic agents for modulating tissue growth by targeting activin and ActRIIA polypeptides. In certain embodiments, high-throughput screening of compounds can be carried out to identify agents that perturb activin or ActRIIA-mediated effects on bone. In certain embodiments, the assay is carried out to screen and identify compounds that specifically inhibit or reduce binding of an ActRIIA polypeptide to activin. Alternatively, the assay can be used to identify compounds that enhance binding of an ActRIIA polypeptide to activin. In a further embodiment, the compounds can be identified by their ability to interact with an activin or ActRIIA polypeptide.

A variety of assay formats will suffice and, in light of the present disclosure, those not expressly described herein will nevertheless be comprehended by one of ordinary skill in the art. As described herein, the test compounds (agents) used herein may be created by any combinatorial chemical method. Alternatively, the subject compounds may be naturally occurring biomolecules synthesized in vivo or in vitro. Compounds (agents) to be tested for their ability to act as modulators of tissue growth can be produced, for example, by bacteria, yeast, plants or other organisms (e.g., natural products), produced chemically (e.g., small molecules, including peptidomimetics), or produced recombinantly. Test compounds contemplated herein include non-peptidyl organic molecules, peptides, polypeptides, peptidomimetics, sugars, hormones, and nucleic acid molecules. In a specific embodiment, the test agent is a small organic molecule having a molecular weight of less than about 2,000 daltons.

The test compounds can be provided as single, discrete entities, or provided in libraries of greater complexity, such as made by combinatorial chemistry. These libraries can comprise, for example, alcohols, alkyl halides, amines, amides, esters, aldehydes, ethers and other classes of organic compounds. Presentation of test compounds to the test system can be in either an isolated form or as mixtures of compounds, especially in initial screening steps. Optionally, the compounds may be derivatized with other compounds and have derivatizing groups that facilitate isolation of the compounds. Non-limiting examples of derivatizing groups include biotin, fluorescein, digoxygenin, green fluorescent protein, isotopes, polyhistidine, magnetic beads, glutathione S transferase (GST), photoactivatable crosslinkers or any combinations thereof.

In many drug screening programs which test libraries of compounds and natural extracts, high throughput assays are desirable in order to maximize the number of compounds surveyed in a given period of time. Assays which are performed in cell-free systems, such as may be derived with purified or semi-purified proteins, are often preferred as “primary” screens in that they can be generated to permit rapid development and relatively easy detection of an alteration in a molecular target which is mediated by a test compound. Moreover, the effects of cellular toxicity or bioavailability of the test compound can be generally ignored in the in vitro system, the assay instead being focused primarily on the effect of the drug on the molecular target as may be manifest in an alteration of binding affinity between an ActRIIA polypeptide and activin.

Merely to illustrate, in an exemplary screening assay, the compound of interest is contacted with an isolated and purified ActRIIA polypeptide which is ordinarily capable of binding to activin. To the mixture of the compound and ActRIIA polypeptide is then added a composition containing an ActRIIA ligand. Detection and quantification of ActRIIA/activin complexes provides a means for determining the compound's efficacy at inhibiting (or potentiating) complex formation between the ActRIIA polypeptide and activin. The efficacy of the compound can be assessed by generating dose response curves from data obtained using various concentrations of the test compound. Moreover, a control assay can also be performed to provide a baseline for comparison. For example, in a control assay, isolated and purified activin is added to a composition containing the ActRIIA polypeptide, and the formation of ActRIIA/activin complex is quantitated in the absence of the test compound. It will be understood that, in general, the order in which the reactants may be admixed can be varied, and can be admixed simultaneously. Moreover, in place of purified proteins, cellular extracts and lysates may be used to render a suitable cell-free assay system.

Complex formation between the ActRIIA polypeptide and activin may be detected by a variety of techniques. For instance, modulation of the formation of complexes can be quantitated using, for example, detectably labeled proteins such as radiolabeled (e.g., 32P, 35S, 14C or 3H), fluorescently labeled (e.g., FITC), or enzymatically labeled ActRIIA polypeptide or activin, by immunoassay, or by chromatographic detection.

In certain embodiments, contemplated herein is the use of fluorescence polarization assays and fluorescence resonance energy transfer (FRET) assays in measuring, either directly or indirectly, the degree of interaction between an ActRIIA polypeptide and its binding protein. Further, other modes of detection, such as those based on optical waveguides (PCT Publication WO 96/26432 and U.S. Pat. No. 5,677,196), surface plasmon resonance (SPR), surface charge sensors, and surface force sensors, are compatible with many embodiments described herein.

Moreover, an interaction trap assay, also known as the “two hybrid assay,” can be used for identifying agents that disrupt or potentiate interaction between an ActRIIA polypeptide and its binding protein. See for example, U.S. Pat. No. 5,283,317; Zervos et al. (1993) Cell 72:223-232; Madura et al. (1993) J Biol Chem 268:12046-12054; Bartel et al. (1993) Biotechniques 14:920-924; and Iwabuchi et al. (1993) Oncogene 8:1693-1696). In a specific embodiment, contemplated herein is the use of reverse two hybrid systems to identify compounds (e.g., small molecules or peptides) that dissociate interactions between an ActRIIA polypeptide and its binding protein. See for example, Vidal and Legrain, (1999) Nucleic Acids Res 27:919-29; Vidal and Legrain, (1999) Trends Biotechnol 17:374-81; and U.S. Pat. Nos. 5,525,490; 5,955,280; and 5,965,368.

In certain embodiments, the subject compounds are identified by their ability to interact with an ActRIIA or activin polypeptide. The interaction between the compound and the ActRIIA or activin polypeptide may be covalent or non-covalent. For example, such interaction can be identified at the protein level using in vitro biochemical methods, including photo-crosslinking, radiolabeled ligand binding, and affinity chromatography (Jakoby W B et al., 1974, Methods in Enzymology 46: 1). In certain cases, the compounds may be screened in a mechanism based assay, such as an assay to detect compounds which bind to an activin or ActRIIA polypeptide. This may include a solid phase or fluid phase binding event. Alternatively, the gene encoding an activin or ActRIIA polypeptide can be transfected with a reporter system (e.g., β-galactosidase, luciferase, or green fluorescent protein) into a cell and screened against the library preferably by a high throughput screening or with individual members of the library. Other mechanism based binding assays may be used, for example, binding assays which detect changes in free energy. Binding assays can be performed with the target fixed to a well, bead or chip or captured by an immobilized antibody or resolved by capillary electrophoresis. The bound compounds may be detected usually using colorimetric or fluorescence or surface plasmon resonance.

### 5.5 Methods of Treatment

Provided herein are methods of treating anemia in a subject in need thereof comprising: administering to a subject an activin receptor type IIB (ActRIIB) ligand trap (such as the ActRIIB ligand traps described in Section 5.4); and administering to the subject fedratinib or a pharmaceutically acceptable salt and/or hydrate thereof (as described in Section 5.3). In certain embodiments, the ActRIIB ligand trap and fedratinib or pharmaceutically acceptable salt and/or hydrate thereof are concomitantly administered. In certain embodiments, the co-administration of the ActRIIB ligand trap and fedratinib or pharmaceutically acceptable salt and/or hydrate thereof is pharmaceutically effective to treat anemia.

In some embodiments, the subject is a subject described in Section 5.6. In some embodiments, the subject is a human. In some embodiments, the subject is a subject diagnosed with myelofibrosis. In some embodiments, the subject is a subject diagnosed with myeloproliferative neoplasms (MPN)-associated myelofibrosis. In certain embodiments, the myelofibrosis is a myeloproliferative neoplasms (MPN)-associated myelofibrosis. In certain embodiments, the myelofibrosis is intermediate or high-risk myeloproliferative neoplasms (MPN)-associated myelofibrosis. In certain embodiments, the myelofibrosis is Primary Myelofibrosis (PMF), Post-Polycythemia Vera Myelofibrosis (post-PV MF), or Post-Essential Thrombocythemia Myelofibrosis (post-ET MF). In certain embodiments, the myelofibrosis is intermediate or high-risk Primary Myelofibrosis (PMF), Post-Polycythemia Vera Myelofibrosis (post-PV MF), or Post-Essential Thrombocythemia Myelofibrosis (post-ET MF). In certain embodiments, the myelofibrosis is intermediate or high-risk primary myelofibrosis (PMF). In certain embodiments, the myelofibrosis is intermediate or high-risk post-polycythemia vera myelofibrosis (post-PV MF). In certain embodiments, the myelofibrosis is intermediate or high-risk post-essential thrombocythemia myelofibrosis (post-ET MF).

In some embodiments, the myeloproliferative neoplasm-associated myelofibrosis is primary myelofibrosis. In some embodiments, the myeloproliferative neoplasm-associated myelofibrosis is polycythemia vera myelofibrosis. In more some embodiments, the polycythemia vera myelofibrosis is associated with, or caused by, a JAK2 mutation, e.g., a JAK2 V617F mutation or JAK2 exon 12 mutation, or a thrombopoietin receptor (MPL) mutation. In some embodiments, the myeloproliferative neoplasm-associated myelofibrosis is post-polycythemia vera myelofibrosis. In more some embodiments, the post-polycythemia vera myelofibrosis is associated with, or caused by, a JAK2 mutation, e.g., a JAK2 V617F mutation, or a thrombopoietin receptor (MPL) mutation. In some embodiments, the myeloproliferative neoplasm-associated myelofibrosis is essential thrombocytopenia or post-essential thrombocythemia myelofibrosis. In some embodiments, the essential thrombocytopenia or post-essential thrombocythemia myelofibrosis is associated with, or caused by, a JAK2 mutation, e.g., a JAK2 V617F mutation, or a thrombopoietin receptor (MPL) mutation. In some embodiments, the essential thrombocytopenia or post-essential thrombocythemia myelofibrosis includes reticulin fibrosis or trichrome fibrosis.

In certain embodiments, the subject has anemia. In certain embodiments, the anemia is associated with myelofibrosis. In certain embodiments, the anemia is associated with MPN-associated myelofibrosis. In certain embodiments, the anemia is associated with Primary Myelofibrosis (PMF), Post-Polycythemia Vera Myelofibrosis (post-PV MF), or Post-Essential Thrombocythemia Myelofibrosis (post-ET MF).

In certain embodiments, the anemia is an anemia associated with ineffective erythropoiesis, thalassemia, alpha-thalassemia, beta-thalassemia, myelodysplastic syndromes (MDS), or non-proliferative chronic myelomonocytic leukemia (CMML).

In some embodiments, the subject is a subject described in Section 5.6. In some embodiments, the subject is a human.

In some embodiments, the subject is red blood cell transfusion dependent or non-transfusion dependent. In certain embodiments, the subject is red blood cell transfusion dependent. In certain embodiments, the subject is non-transfusion dependent. In certain embodiments, the subject is red blood cell transfusion-independent.

In some embodiments, as used herein, “transfusion” refers to red blood cell (RBC) transfusion.

In some embodiments, the subject is non-transfusion dependent or red blood cell transfusion-independent if the subject has received less than 4 RBC units in RBC transfusion during a period of time of 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days prior to administration of fedratinib and/or the ActRIIB ligand trap to the subject. In some embodiments, the subject is non-transfusion dependent or RBC transfusion-independent if the subject has received 1, 2, or 3 RBC units in RBC transfusion during a period of time of 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days prior to administration of fedratinib and/or the ActRIIB ligand trap to the subject. In some embodiments, the subject is non-transfusion dependent or RBC transfusion-independent if the subject has received no (0) RBC units during a period of time of 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days prior to administration of fedratinib and/or the ActRIIB ligand trap to the subject. In some embodiments, the subject is non-transfusion dependent or RBC transfusion-independent if the subject has received no (0) RBC transfusion during a period of time of 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days prior to administration of fedratinib and/or the ActRIIB ligand trap to the subject.

In one embodiment, the subject is non-transfusion dependent or RBC transfusion-independent if the subject has received less than 4 RBC units in RBC transfusion during a period of time of 84 days prior to administration of fedratinib and/or the ActRIIB ligand trap to the subject. In one embodiment, the subject is non-transfusion dependent or RBC transfusion-independent if the subject has received 1, 2, or 3 RBC units in RBC transfusion during a period of time of 84 days prior to administration of fedratinib and/or the ActRIIB ligand trap to the subject. In one embodiment, the subject is non-transfusion dependent or RBC transfusion-independent if the subject has received no (0) RBC units in RBC transfusion during a period of time of 84 days prior to administration of fedratinib and/or the ActRIIB ligand trap to the subject. In one embodiment, the subject is non-transfusion dependent or RBC transfusion-independent if the subject has received no (0) RBC transfusion during a period of time of 84 days prior to administration of fedratinib and/or the ActRIIB ligand trap to the subject.

In one embodiment, the non-transfusion dependent or RBC transfusion-independent subject has received less than 4 RBC units in RBC transfusion during a period of time of 84 days prior to administration of fedratinib and/or the ActRIIB ligand trap to the subject, and the subject has a hemoglobin (Hgb) level of less than 9.5 g/dL. In another embodiment, the non-transfusion dependent or RBC transfusion-independent subject has received no (0) RBC units in RBC transfusion during a period of time of 84 days prior to administration of fedratinib and/or the ActRIIB ligand trap to the subject, and the subject has a hemoglobin (Hgb) level of less than 9.5 g/dL. In yet another embodiment, the non-transfusion dependent or RBC transfusion-independent subject has received no (0) RBC transfusion during a period of time of 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days prior to administration of fedratinib and/or the ActRIIB ligand trap to the subject, and the subject has a hemoglobin (Hgb) level of less than 9.5 g/dL.

In some embodiments, the subject is red blood cell transfusion dependent if the subject has received 4 or more RBC units during a period of time of at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days prior to administration of fedratinib and/or the ActRIIB ligand trap to the subject. In some embodiments, the subject is red blood cell transfusion dependent if the subject has received 4, 5, 6, 7, 8, 9, 10, 11, 12, 13, 14, 15, 16, 17, 18, 19, or 20 RBC units during a period of time of at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days prior to administration of fedratinib and/or the ActRIIB ligand trap to the subject. In some embodiments, the subject is red blood cell transfusion dependent if the subject has received 4 to 16 RBC units during a period of time of at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days prior to administration of fedratinib and/or the ActRIIB ligand trap to the subject. In some embodiments, the subject is red blood cell transfusion dependent if the subject has received 4 to 12 RBC units during a period of at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days prior to administration of fedratinib and/or the ActRIIB ligand trap to the subject. In some embodiments, the subject is red blood cell transfusion dependent if the subject has received 4 to 10 RBC units during a period of at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days prior to administration of fedratinib and/or the ActRIIB ligand trap to the subject. In some embodiments, the subject is red blood cell transfusion dependent if the subject has received 4 to 8 RBC units during a period of at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days prior to administration of fedratinib and/or the ActRIIB ligand trap to the subject. In some embodiments, the subject is red blood cell transfusion dependent if the subject has received 4 to 6 RBC units during a period of at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days prior to administration of fedratinib and/or the ActRIIB ligand trap to the subject.

In some embodiments, the subject is red blood cell transfusion dependent if the subject has received 4 or more RBC units during a period of time of 84 days prior to administration of fedratinib and/or the ActRIIB ligand trap to the subject. In some embodiments, the subject is red blood cell transfusion dependent if the subject has received 4, 5, 6, 7, 8, 9, 10, 11, 12, 13, 14, 15, 16, 17, 18, 19, or 20 RBC units during a period of time of 84 days prior to administration of fedratinib and/or the ActRIIB ligand trap to the subject. In some embodiments, the subject is red blood cell transfusion dependent if the subject has received 4 to 16 RBC units during a period of time of 84 days prior to administration of fedratinib and/or the ActRIIB ligand trap to the subject. In some embodiments, the subject is red blood cell transfusion dependent if the subject has received 4 to 12 RBC units during a period of 84 days prior to administration of fedratinib and/or the ActRIIB ligand trap to the subject. In some embodiments, the subject is red blood cell transfusion dependent if the subject has received 4 to 10 RBC units during a period of 84 days prior to administration of fedratinib and/or the ActRIIB ligand trap to the subject. In some embodiments, the subject is red blood cell transfusion dependent if the subject has received 4 to 8 RBC units during a period of 84 days prior to administration of fedratinib and/or the ActRIIB ligand trap to the subject. In some embodiments, the subject is red blood cell transfusion dependent if the subject has received 4 to 6 RBC units during a period of 84 days prior to administration of fedratinib and/or the ActRIIB ligand trap to the subject. In some embodiments, the subject is red blood cell transfusion dependent if the subject has received 6 to 12, 6 to 10, or 6 to 8 RBC units during a period of 84 days prior to administration of fedratinib and/or the ActRIIB ligand trap to the subject. In some embodiments, the subject is red blood cell transfusion dependent if the subject has received 8 to 12 or 8 to 10 RBC units during a period of 84 days prior to administration of fedratinib and/or the ActRIIB ligand trap to the subject.

In one embodiment, the red blood cell transfusion dependent subject has received 4 or more RBC units during a period of time of at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days prior to administration of fedratinib and/or the ActRIIB ligand trap to the subject, and the subject has a hemoglobin (Hgb) level of less than 11.5 g/dL. In another embodiment, the red blood cell transfusion dependent subject has received 4 to 12 RBC units during a period of at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days prior to administration of fedratinib and/or the ActRIIB ligand trap to the subject, and the subject has a hemoglobin (Hgb) level of less than 11.5 g/dL.

In one embodiments, the RBC transfusion dependent subject has received 4 or more red blood cell units during a period of time of 84 days prior to administration of fedratinib and/or the ActRIIB ligand trap to the subject, and the subject has a hemoglobin (Hgb) level of less than 11.5 g/dL. In another embodiments, the RBC dependent subject has received 4 to 12 red blood cell units during a period of 84 days prior to administration of fedratinib and/or the ActRIIB ligand trap to the subject, and the subject has a hemoglobin (Hgb) level of less than 11.5 g/dL.

In some embodiments, the RBC transfusion dependent subject has no interval of greater than 2, 3, 4, 5, 6, 7, or 8 weeks without RBC transfusion during a period of time of of 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days prior to administration of fedratinib and/or the ActRIIB ligand trap to the subject. In some embodiments, the RBC transfusion dependent subject has no interval of greater than 4, 5, 6, or 7 weeks without RBC transfusion during a period of time of 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days prior to administration of fedratinib and/or the ActRIIB ligand trap to the subject. In some embodiments, the RBC transfusion dependent subject has no interval of greater than 6 weeks without RBC transfusion during a period of time of 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days prior to administration of fedratinib and/or the ActRIIB ligand trap to the subject. In some embodiments, the RBC transfusion dependent subject has no interval of greater than 2, 3, 4, 5, 6, 7, or 8 weeks without RBC transfusion during a period of time of 84 days prior to administration of fedratinib and/or the ActRIIB ligand trap to the subject. In some embodiments, the RBC transfusion dependent subject has no interval of greater than 4, 5, 6, or 7 weeks without RBC transfusion during a period of time of 84 days prior to administration of fedratinib and/or the ActRIIB ligand trap to the subject. In some embodiments, the RBC transfusion dependent subject has no interval of greater than 6 weeks without RBC transfusion during a period of time of 84 days prior to administration of fedratinib and/or the ActRIIB ligand trap to the subject.

In one embodiment, the RBC dependent subject has received 4 or more RBC units in RBC transfusion and has no interval of great than 2, 3, 4, 5, 6, 7, or 8 weeks without RBC transfusion during a period of time of 84 days prior to administration of fedratinib and/or the ActRIIB ligand trap to the subject, and the RBC dependent subject has a hemoglobin (Hgb) level of less than 11.5 g/dL. In another embodiment, the RBC dependent subject has received 4 to 12 RBC units in RBC transfusion and has no interval of great than 2, 3, 4, 5, 6, 7, or 8 weeks without RBC transfusion during a period of time of 84 days prior to administration of fedratinib and/or the ActRIIB ligand trap to the subject, and the RBC dependent subject has a hemoglobin (Hgb) level of less than 11.5 g/dL. In yet another embodiment, the RBC dependent subject has received 4 or more RBC units in RBC transfusion and has no interval of great than 6 weeks without RBC transfusion during a period of time of 84 days prior to administration of fedratinib thereof and/or the ActRIIB ligand trap to the subject, and the RBC dependent subject has a hemoglobin (Hgb) level of less than 11.5 g/dL. In yet another embodiment, the RBC dependent subject has received 4 to 12 RBC units in RBC transfusion and has no interval of great than 6 weeks without RBC transfusion during a period of time of 84 days prior to administration of fedratinib and/or the ActRIIB ligand trap to the subject, and the RBC dependent subject has a hemoglobin (Hgb) level of less than 11.5 g/dL.

In some embodiments, fedratinib is administered to the subject as part of a composition. In some embodiments, the composition is a composition described in Section 5.7.

In some embodiments, fedratinib is administered at a dose described in Section 5.8. In one embodiment, fedratinib is administered at a dose of 400 mg/day, 300 mg/day, 200 mg/day, or 100 mg/day. In another embodiment, fedratinib is administered to the subject at frequency described in Section 5.8. In preferred embodiments, fedratinib is administered daily.

In some embodiments, fedratinib is administered to the subject via a route of administration described in Section 5.10. In preferred embodiments, fedratinib is administered to the subject orally.

In some embodiments, the ActRIIB ligand trap is administered to the subject as part of a composition. In some embodiments, the composition is a composition described in Section 5.7.

In some embodiments, the ActRIIB ligand trap is administered to the subject via a route of administration described in Section 5.10. In some embodiments, the ActRIIB ligand trap is administered to the subject intravenously or subcutaneously. In preferred embodiments, the ActRIIB ligand trap is administered to the subject subcutaneously.

In some embodiments, fedratinib is administered at a dose described in Section 5.8. In some embodiments, the ActRIIB ligand trap is administered at a dose of from 0.3 mg/kg to 2.0 mg/kg, e.g., 0.3 mg/kg, 0.33 mg/kg, 0.45 mg/kg, 0.6 mg/kg, 0.8 mg/kg, 1.0 mg/kg, 1.33 mg/kg, 1.75 mg/kg, or 2.0 mg/kg. In some embodiments, the ActRIIB ligand trap is administered at a pharmaceutically effective amount. In one embodiment, the pharmaceutically effective amount of the ActRIIB ligand trap administered is 1.33 mg/kg. In some embodiments, the ActRIIB ligand trap is administered to the subject at a frequency described in Section 5.8. In some embodiments, the ActRIIB ligand trap is administered to the subject once every 14 days, 21 days, once every 28 days, every 35 days, or once every 42 days. In preferred embodiments, the ActRIIB ligand trap is administered to the subject once every 21 days. In some embodiments, the ActRIIB ligand trap is administered to the subject 1, 2, or 3 times every treatment cycle. In preferred embodiments, the ActRIIB ligand trap is administered to the subject once every treatment cycle. In some embodiments, the ActRIIB ligand trap is administered to the subject for at least 1, 2, 3, 4, 5, 6, 7, 8, 9, or 10 cycles. In one embodiment, the ActRIIB ligand trap is administered to the subject for at least 6, 7, 8, 9, or 10 cycles. In another embodiment, the ActRIIB ligand trap is administered to the subject for 6 cycles. In yet another embodiment, the ActRIIB ligand trap is administered to the subject for 7 cycles. In yet another embodiment, the ActRIIB ligand trap is administered to the subject for 8 cycles. In yet another embodiment, the ActRIIB ligand trap is administered to the subject for 9 cycles. In yet another embodiment, the ActRIIB ligand trap is administered to the subject for 10 cycles. In one embodiment, the ActRIIB ligand trap is administered once at the beginning of every treatment cycle.

In some embodiments, the ActRIIB ligand trap is an ActRIIB ligand trap described in Section 5.4. In some embodiments, the ActRIIB ligand trap is a polypeptide comprising the amino acid sequence of SEQ ID NO:11. In some embodiments, the ActRIIB ligand trap is a polypeptide comprising an amino acid sequence consisting of the amino acid sequence of SEQ ID NO:11. In some embodiments, the ActRIIB ligand trap is a polypeptide consisting of the amino acid sequence set forth in SEQ ID NO:11. In some embodiments, the ActRIIB ligand trap is a polypeptide is encoded by the nucleotide sequence of SEQ ID NO:34 or a degenerate version of SEQ ID NO:34. In a preferred embodiment, the ActRIIB ligand trap is luspatercept.

In certain embodiments, the ActRIIB ligand trap is a polypeptide comprising an amino acid sequence selected from the group consisting of: (a) 90% identical to SEQ ID NO:3; (b) 95% identical to SEQ ID NO:3; (c) 98% identical to SEQ ID NO:3; (d) SEQ ID NO:3; (e) 90% identical to SEQ ID NO:6; (f) 95% identical to SEQ ID NO:6; (g) 98% identical to SEQ ID NO:6; (h) SEQ ID NO:6; (i) 90% identical to SEQ ID NO:7; (j) 95% identical to SEQ ID NO:7; (k) 98% identical to SEQ ID NO:7; (1) SEQ ID NO:7; (m) 90% identical to SEQ ID NO:11; (n) 95% identical to SEQ ID NO:11; (o) 98% identical to SEQ ID NO:11; and (p) SEQ ID NO:11. In certain embodiments, the ActRIIB ligand trap is a product resulting from expression from an opening reading frame with the nucleotide sequence of SEQ ID NO:34 or a degenerate version of SEQ ID NO:34 that encodes SEQ ID NO:11. In certain embodiments, the ActRIIB ligand trap is a humanized fusion-protein consisting of the extracellular domain of ActRIIB and the human IgG1 Fc domain.

Also provided herein are methods of treating in a subject in need thereof, comprising: (a) taking a first measurement of hemoglobin (Hgb) level in a subject; (b) administering to the subject an initial dose of an ActRIIB ligand trap (such as the ActRIIB ligand inhibitors described in Section 5.4); (c) administering to the subject fedratinib or a pharmaceutically acceptable salt and/or hydrate thereof (as described in Section 5.3); (d) taking a second measurement of hemoglobin (Hgb) level in the subject at the end of a first period of time after the administration of the initial dose of an ActRIIB ligand trap; and (e) administering to the subject a subsequent dose of the ActRIIB ligand trap based on the second measurement of hemoglobin (Hgb) level as compared to the first measurement of hemoglobin (Hgb) level, or based on number of red blood cell transfusion that the subject received during the first period of time. In certain embodiments, the co-administration of the ActRIIB ligand trap and fedratinib or pharmaceutically acceptable salt or hydrate thereof is pharmaceutically effective to treatment anemia.

In some embodiments, the subject is a subject described in Section 5.6. In some embodiments, the subject is a human. In some embodiments, the subject is a subject diagnosed with myelofibrosis. In some embodiments, the subject is a subject diagnosed with myeloproliferative neoplasms (MPN)-associated myelofibrosis. In certain embodiments, the myelofibrosis is a myeloproliferative neoplasms (MPN)-associated myelofibrosis. In certain embodiments, the myelofibrosis is intermediate or high-risk myeloproliferative neoplasms (MPN)-associated myelofibrosis. In certain embodiments, the myelofibrosis is Primary Myelofibrosis (PMF), Post-Polycythemia Vera Myelofibrosis (post-PV MF), or Post-Essential Thrombocythemia Myelofibrosis (post-ET MF). In certain embodiments, the myelofibrosis is intermediate or high-risk Primary Myelofibrosis (PMF), Post-Polycythemia Vera Myelofibrosis (post-PV MF), or Post-Essential Thrombocythemia Myelofibrosis (post-ET MF). In certain embodiments, the myelofibrosis is intermediate or high-risk primary myelofibrosis (PMF). In certain embodiments, the myelofibrosis is intermediate or high-risk post-polycythemia vera myelofibrosis (post-PV MF). In certain embodiments, the myelofibrosis is intermediate or high-risk post-essential thrombocythemia myelofibrosis (post-ET MF).

In some embodiments, the myeloproliferative neoplasm-associated myelofibrosis is primary myelofibrosis. In some embodiments, the myeloproliferative neoplasm-associated myelofibrosis is polycythemia vera myelofibrosis. In more some embodiments, the polycythemia vera myelofibrosis is associated with, or caused by, a JAK2 mutation, e.g., a JAK2 V617F mutation or JAK2 exon 12 mutation, or a thrombopoietin receptor (MPL) mutation. In some embodiments, the myeloproliferative neoplasm-associated myelofibrosis is post-polycythemia vera myelofibrosis. In more some embodiments, the post-polycythemia vera myelofibrosis is associated with, or caused by, a JAK2 mutation, e.g., a JAK2 V617F mutation, or a thrombopoietin receptor (MPL) mutation. In some embodiments, the myeloproliferative neoplasm-associated myelofibrosis is essential thrombocytopenia or post-essential thrombocythemia myelofibrosis. In more some embodiments, the essential thrombocytopenia or post-essential thrombocythemia myelofibrosis is associated with, or caused by, a JAK2 mutation, e.g., a JAK2 V617F mutation, or a thrombopoietin receptor (MPL) mutation. In more some embodiments, the essential thrombocytopenia or post-essential thrombocythemia myelofibrosis includes reticulin fibrosis or trichrome fibrosis.

In certain embodiments, the subject has anemia. In certain embodiments, the anemia is associated with myelofibrosis. In certain embodiments, the anemia is associated with MPN-associated myelofibrosis. In certain embodiments, the anemia is associated with Primary Myelofibrosis (PMF), Post-Polycythemia Vera Myelofibrosis (post-PV MF), or Post-Essential Thrombocythemia Myelofibrosis (post-ET MF).

In certain embodiments, the anemia is an anemia associated with ineffective erythropoiesis, thalassemia, alpha-thalassemia, beta-thalassemia, myelodysplastic syndromes (MDS), or non-proliferative chronic myelomonocytic leukemia (CMML).

In some embodiments, the subject is a subject described in Section 5.6. In some embodiments, the subject is a human.

In some embodiments, the subject is red blood cell transfusion dependent or non-transfusion dependent. In certain embodiments, the subject is red blood cell transfusion dependent. In certain embodiments, the subject is non-transfusion dependent. In certain embodiments, the subject is red blood cell transfusion-independent.

In some embodiments, as used herein, “transfusion” refers to red blood cell (RBC) transfusion.

In some embodiments, the subject is non-transfusion dependent or red blood cell transfusion-independent if the subject has received less than 4 RBC units in RBC transfusion during a period of time of 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days prior to administration of fedratinib and/or the ActRIIB ligand trap to the subject. In some embodiments, the subject is non-transfusion dependent or RBC transfusion-independent if the subject has received 1, 2, or 3 RBC units in RBC transfusion during a period of time of 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days prior to administration of fedratinib and/or the ActRIIB ligand trap to the subject. In some embodiments, the subject is non-transfusion dependent or RBC transfusion-independent if the subject has received no (0) RBC units during a period of time of 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days prior to administration of fedratinib and/or the ActRIIB ligand trap to the subject. In some embodiments, the subject is non-transfusion dependent or RBC transfusion-independent if the subject has received no (0) RBC transfusion during a period of time of 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days prior to administration of fedratinib and/or the ActRIIB ligand trap to the subject.

In one embodiment, the subject is non-transfusion dependent or RBC transfusion-independent if the subject has received less than 4 RBC units in RBC transfusion during a period of time of 84 days prior to administration of fedratinib and/or the ActRIIB ligand trap to the subject. In one embodiment, the subject is non-transfusion dependent or RBC transfusion-independent if the subject has received 1, 2, or 3 RBC units in RBC transfusion during a period of time of 84 days prior to administration of fedratinib and/or the ActRIIB ligand trap to the subject. In one embodiment, the subject is non-transfusion dependent or RBC transfusion-independent if the subject has received no (0) RBC units in RBC transfusion during a period of time of 84 days prior to administration of fedratinib and/or the ActRIIB ligand trap to the subject. In one embodiment, the subject is non-transfusion dependent or RBC transfusion-independent if the subject has received no (0) RBC transfusion during a period of time of 84 days prior to administration of fedratinib and/or the ActRIIB ligand trap to the subject.

In one embodiment, the non-transfusion dependent or RBC transfusion-independent subject has received less than 4 RBC units in RBC transfusion during a period of time of 84 days prior to administration of fedratinib and/or the ActRIIB ligand trap to the subject, and the subject has a hemoglobin (Hgb) level of less than 9.5 g/dL. In another embodiment, the non-transfusion dependent or RBC transfusion-independent subject has received no (0) RBC units in RBC transfusion during a period of time of 84 days prior to administration of fedratinib and/or the ActRIIB ligand trap to the subject, and the subject has a hemoglobin (Hgb) level of less than 9.5 g/dL. In yet another embodiment, the non-transfusion dependent or RBC transfusion-independent subject has received no (0) RBC transfusion during a period of time of 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days prior to administration of fedratinib and/or the ActRIIB ligand trap to the subject, and the subject has a hemoglobin (Hgb) level of less than 9.5 g/dL.

In some embodiments, the subject is red blood cell transfusion dependent if the subject has received 4 or more RBC units during a period of time of at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days prior to administration of fedratinib and/or the ActRIIB ligand trap to the subject. In some embodiments, the subject is red blood cell transfusion dependent if the subject has received 4, 5, 6, 7, 8, 9, 10, 11, 12, 13, 14, 15, 16, 17, 18, 19, or 20 RBC units during a period of time of at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days prior to administration of fedratinib and/or the ActRIIB ligand trap to the subject. In some embodiments, the subject is red blood cell transfusion dependent if the subject has received 4 to 16 RBC units during a period of time of at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days prior to administration of fedratinib and/or the ActRIIB ligand trap to the subject. In some embodiments, the subject is red blood cell transfusion dependent if the subject has received 4 to 12 RBC units during a period of at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days prior to administration of fedratinib and/or the ActRIIB ligand trap to the subject. In some embodiments, the subject is red blood cell transfusion dependent if the subject has received 4 to 10 RBC units during a period of at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days prior to administration of fedratinib and/or the ActRIIB ligand trap to the subject. In some embodiments, the subject is red blood cell transfusion dependent if the subject has received 4 to 8 RBC units during a period of at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days prior to administration of fedratinib and/or the ActRIIB ligand trap to the subject. In some embodiments, the subject is red blood cell transfusion dependent if the subject has received 4 to 6 RBC units during a period of at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days prior to administration of fedratinib and/or the ActRIIB ligand trap to the subject.

In some embodiments, the subject is red blood cell transfusion dependent if the subject has received 4 or more RBC units during a period of time of 84 days prior to administration of fedratinib and/or the ActRIIB ligand trap to the subject. In some embodiments, the subject is red blood cell transfusion dependent if the subject has received 4, 5, 6, 7, 8, 9, 10, 11, 12, 13, 14, 15, 16, 17, 18, 19, or 20 RBC units during a period of time of 84 days prior to administration of fedratinib and/or the ActRIIB ligand trap to the subject. In some embodiments, the subject is red blood cell transfusion dependent if the subject has received 4 to 16 RBC units during a period of time of 84 days prior to administration of fedratinib and/or the ActRIIB ligand trap to the subject. In some embodiments, the subject is red blood cell transfusion dependent if the subject has received 4 to 12 RBC units during a period of 84 days prior to administration of fedratinib and/or the ActRIIB ligand trap to the subject. In some embodiments, the subject is red blood cell transfusion dependent if the subject has received 4 to 10 RBC units during a period of 84 days prior to administration of fedratinib and/or the ActRIIB ligand trap to the subject. In some embodiments, the subject is red blood cell transfusion dependent if the subject has received 4 to 8 RBC units during a period of 84 days prior to administration of fedratinib and/or the ActRIIB ligand trap to the subject. In some embodiments, the subject is red blood cell transfusion dependent if the subject has received 4 to 6 RBC units during a period of 84 days prior to administration of fedratinib and/or the ActRIIB ligand trap to the subject. In some embodiments, the subject is red blood cell transfusion dependent if the subject has received 6 to 12, 6 to 10, or 6 to 8 RBC units during a period of 84 days prior to administration of fedratinib and/or the ActRIIB ligand trap to the subject. In some embodiments, the subject is red blood cell transfusion dependent if the subject has received 8 to 12 or 8 to 10 RBC units during a period of 84 days prior to administration of fedratinib and/or the ActRIIB ligand trap to the subject.

In one embodiment, the red blood cell transfusion dependent subject has received 4 or more RBC units during a period of time of at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days prior to administration of fedratinib and/or the ActRIIB ligand trap to the subject, and the subject has a hemoglobin (Hgb) level of less than 11.5 g/dL. In another embodiment, the red blood cell transfusion dependent subject has received 4 to 12 RBC units during a period of at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days prior to administration of fedratinib and/or the ActRIIB ligand trap to the subject, and the subject has a hemoglobin (Hgb) level of less than 11.5 g/dL.

In one embodiments, the RBC transfusion dependent subject has received 4 or more red blood cell units during a period of time of 84 days prior to administration of fedratinib and/or the ActRIIB ligand trap to the subject, and the subject has a hemoglobin (Hgb) level of less than 11.5 g/dL. In another embodiments, the RBC dependent subject has received 4 to 12 red blood cell units during a period of 84 days prior to administration of fedratinib and/or the ActRIIB ligand trap to the subject, and the subject has a hemoglobin (Hgb) level of less than 11.5 g/dL.

In some embodiments, the RBC transfusion dependent subject has no interval of greater than 2, 3, 4, 5, 6, 7, or 8 weeks without RBC transfusion during a period of time of 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days prior to administration of fedratinib and/or the ActRIIB ligand trap to the subject. In some embodiments, the RBC transfusion dependent subject has no interval of greater than 4, 5, 6, or 7 weeks without RBC transfusion during a period of time of 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days prior to administration of fedratinib and/or the ActRIIB ligand trap to the subject. In some embodiments, the RBC transfusion dependent subject has no interval of greater than 6 weeks without RBC transfusion during a period of time of 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days prior to administration of fedratinib and/or the ActRIIB ligand trap to the subject. In some embodiments, the RBC transfusion dependent subject has no interval of greater than 2, 3, 4, 5, 6, 7, or 8 weeks without RBC transfusion during a period of time of 84 days prior to administration of fedratinib and/or the ActRIIB ligand trap to the subject. In some embodiments, the RBC transfusion dependent subject has no interval of greater than 4, 5, 6, or 7 weeks without RBC transfusion during a period of time of 84 days prior to administration of fedratinib and/or the ActRIIB ligand trap to the subject. In some embodiments, the RBC transfusion dependent subject has no interval of greater than 6 weeks without RBC transfusion during a period of time of 84 days prior to administration of fedratinib and/or the ActRIIB ligand trap to the subject.

In one embodiment, the RBC dependent subject has received 4 or more RBC units in RBC transfusion and has no interval of great than 2, 3, 4, 5, 6, 7, or 8 weeks without RBC transfusion during a period of time of 84 days prior to administration of fedratinib and/or the ActRIIB ligand trap to the subject, and the RBC dependent subject has a hemoglobin (Hgb) level of less than 11.5 g/dL. In another embodiment, the RBC dependent subject has received 4 to 12 RBC units in RBC transfusion and has no interval of great than 2, 3, 4, 5, 6, 7, or 8 weeks without RBC transfusion during a period of time of 84 days prior to administration of fedratinib and/or the ActRIIB ligand trap to the subject, and the RBC dependent subject has a hemoglobin (Hgb) level of less than 11.5 g/dL. In yet another embodiment, the RBC dependent subject has received 4 or more RBC units in RBC transfusion and has no interval of great than 6 weeks without RBC transfusion during a period of time of 84 days prior to administration of fedratinib thereof and/or the ActRIIB ligand trap to the subject, and the RBC dependent subject has a hemoglobin (Hgb) level of less than 11.5 g/dL. In yet another embodiment, the RBC dependent subject has received 4 to 12 RBC units in RBC transfusion and has no interval of great than 6 weeks without RBC transfusion during a period of time of 84 days prior to administration of fedratinib and/or the ActRIIB ligand trap to the subject, and the RBC dependent subject has a hemoglobin (Hgb) level of less than 11.5 g/dL.

In some embodiments, fedratinib is administered to the subject as part of a composition. In some embodiments, the composition is a composition described in Section 5.7.

In some embodiments, fedratinib is administered at a dose described in Section 5.8. In one embodiment, fedratinib is administered at a dose of 600 mg/day, 500 mg/day, 400 mg/day, 300 mg/day, 200 mg/day, or 100 mg/day. In another embodiment, fedratinib is administered to the subject at frequency described in Section 5.8. In preferred embodiments, fedratinib is administered daily.

In some embodiments, fedratinib is administered to the subject via a route of administration described in Section 5.10. In preferred embodiments, fedratinib is administered to the subject orally.

In some embodiments, the ActRIIB ligand trap is administered to the subject as part of a composition. In some embodiments, the composition is a composition described in Section 5.7.

In some embodiments, the ActRIIB ligand trap is administered to the subject via a route of administration described in Section 5.10. In some embodiments, the ActRIIB ligand trap is administered to the subject intravenously or subcutaneously. In preferred embodiments, the ActRIIB ligand trap is administered to the subject subcutaneously.

In some embodiments, fedratinib is administered at a dose described in Section 5.8. In some embodiments, the ActRIIB ligand trap is administered at a dose of from 0.3 mg/kg to 2.0 mg/kg, e.g., 0.33 mg/kg, 0.45 mg/kg, 0.6 mg/kg, 0.8 mg/kg, 1.0 mg/kg, 1.33 mg/kg, 1.75 mg/kg, or 2.0 mg/kg. In some embodiments, the ActRIIB ligand trap is administered at a pharmaceutically effective amount. In one embodiment, the pharmaceutically effective amount of the ActRIIB ligand trap administered is 1.33 mg/kg. In some embodiments, the ActRIIB ligand trap is administered to the subject at a frequency described in Section 5.8. In some embodiments, the ActRIIB ligand trap is administered to the subject once every 14 days, 21 days, once every 28 days, every 35 days, or once every 42 days. In preferred embodiments, the ActRIIB ligand trap is administered to the subject once every 21 days. In some embodiments, the ActRIIB ligand trap is administered to the subject 1, 2, or 3 times every treatment cycle. In preferred embodiments, the ActRIIB ligand trap is administered to the subject once every treatment cycle. In some embodiments, the ActRIIB ligand trap is administered to the subject for at least 1, 2, 3, 4, 5, 6, 7, 8, 9, or 10 cycles. In one embodiment, the ActRIIB ligand trap is administered to the subject for at least 6, 7, 8, 9, or 10 cycles. In another embodiment, the ActRIIB ligand trap is administered to the subject for 6 cycles. In yet another embodiment, the ActRIIB ligand trap is administered to the subject for 7 cycles. In yet another embodiment, the ActRIIB ligand trap is administered to the subject for 8 cycles. In yet another embodiment, the ActRIIB ligand trap is administered to the subject for 9 cycles. In yet another embodiment, the ActRIIB ligand trap is administered to the subject for 10 cycles. In one embodiment, the ActRIIB ligand trap is administered once at the beginning of every treatment cycle.

In some embodiments, the ActRIIB ligand trap is an ActRIIB ligand trap described in Section 5.4. In some embodiments, the ActRIIB ligand trap is a polypeptide comprising the amino acid sequence of SEQ ID NO:11. In some embodiments, the ActRIIB ligand trap is a polypeptide comprising an amino acid sequence consisting of the amino acid sequence of SEQ ID NO:11. In some embodiments, the ActRIIB ligand trap is a polypeptide consisting of the amino acid sequence set forth in SEQ ID NO:11. In some embodiments, the ActRIIB ligand trap is a polypeptide is encoded by the nucleotide sequence of SEQ ID NO:34 or a degenerate version of SEQ ID NO:34. In a preferred embodiment, the ActRIIB ligand trap is luspatercept.

In certain embodiments, the ActRIIB ligand trap is a polypeptide comprising an amino acid sequence selected from the group consisting of: (a) 90% identical to SEQ ID NO:3; (b) 95% identical to SEQ ID NO:3; (c) 98% identical to SEQ ID NO:3; (d) SEQ ID NO:3; (e) 90% identical to SEQ ID NO:6; (f) 95% identical to SEQ ID NO:6; (g) 98% identical to SEQ ID NO:6; (h) SEQ ID NO:6; (i) 90% identical to SEQ ID NO:7; (j) 95% identical to SEQ ID NO:7; (k) 98% identical to SEQ ID NO:7; (1) SEQ ID NO:7; (m) 90% identical to SEQ ID NO:11; (n) 95% identical to SEQ ID NO:11; (o) 98% identical to SEQ ID NO:11; and (p) SEQ ID NO:11. In certain embodiments, the ActRIIB ligand trap is a product resulting from expression from an opening reading frame with the nucleotide sequence of SEQ ID NO:34 or a degenerate version of SEQ ID NO:34 that encodes SEQ ID NO:11. In certain embodiments, the ActRIIB ligand trap is a humanized fusion-protein consisting of the extracellular domain of ActRIIB and the human IgG1 Fc domain.

### 5.5.1 Dose Adjustment or Delay of ActRIIB Ligand Trap

In some embodiments, in the methods described herein, the dose of the ActRIIB ligand trap is adjusted during treatment of the subject. In some embodiments, the dose of the ActRIIB ligand trap is adjusted prior to each treatment cycle. In some embodiments, the dose of the ActRIIB ligand trap is adjusted based on a first measurement and a second measurement of hemoglobin (Hgb) level in the subject. In some embodiments, the dose of the ActRIIB ligand trap is adjusted based on In one embodiment, the dose of the ActRIIB ligand trap is adjusted based on comparing the first measurement of hemoglobin (Hgb) level taken in the prior two treatment cycles and the second measurement of hemoglobin (Hgb) level.

In some embodiments, the first measurement of hemoglobin (Hgb) level is taken prior to the administration of the initial dose of the ActRIIB ligand trap. In some embodiments, the first measurement of hemoglobin (Hgb) level is taken prior to each treatment cycle. In some embodiments, the first measurement of hemoglobin (Hgb) level is taken prior to the 1st, 2nd, 3rd, 4th, 5th, 6th, 7th, 8th, 9th, or 10th treatment cycle. In some embodiments, the first measurement of hemoglobin (Hgb) level is taken concurrently with the administration of the initial dose of the ActRIIB ligand trap or taken about 3 weeks, 6 weeks, 9 weeks, 12 weeks, 15 weeks, 18 weeks, 21 weeks, or 24 weeks after the administration of the initial dose of the ActRIIB ligand trap. In some embodiments, the first measurement of hemoglobin (Hgb) level is taken concurrently with the administration of the initial dose of the ActRIIB ligand trap. In some embodiments, the first measurement of hemoglobin (Hgb) level is taken about 3 weeks, 6 weeks, 9 weeks, 12 weeks, 15 weeks, 18 weeks, 21 weeks, or 24 weeks after the administration of the initial dose of the ActRIIB ligand trap.

In some embodiments, the first period of time is 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 15 weeks, 18 weeks, 21 weeks, 24 weeks, 7 months, 8 months, 9 months, 10 months, 11 months, or 12 months. In some embodiments, the first period of time is 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 15 weeks, 18 weeks, 21 weeks, or 24 weeks. In some embodiments, the first period of time is 3 or 6 weeks. In certain embodiments, the first period of time is 3 weeks. In certain embodiments, the first period of time is 6 weeks.

In some embodiments, the second measurement of Hgb level is taken about 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 15 weeks, 18 weeks, 21 weeks, 24 weeks, 7 months, 8 months, 9 months, 10 months, 11 months, or 12 months after the first measurement of Hgb level in the subject. In some embodiments, the second measurement of Hgb level is taken about 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 15 weeks, 18 weeks, or 21 weeks after the first measurement of Hgb level in the subject. In some embodiments, the second measurement of Hgb level is taken about 3 or 6 weeks after the first measurement of Hgb level in the subject. In some embodiments, the second measurement of Hgb level is taken about 3 weeks after the first measurement of Hgb level in the subject. In some embodiments, the second measurement of Hgb level is taken about 6 weeks after the first measurement of Hgb level in the subject.

In some embodiments, the second measurement of hemoglobin (Hgb) level is taken prior to the administration of the initial dose of the ActRIIB ligand trap. In some embodiments, the second measurement of hemoglobin (Hgb) level is taken prior to each treatment cycle starting from the 2nd treatment cycle. In some embodiments, the second measurement of hemoglobin (Hgb) level is taken prior to the 2nd, 3rd, 4th, 5th, 6th, 7th, 8th, 9th, or 10th treatment cycle. In some embodiments, the second measurement of hemoglobin (Hgb) level is taken concurrently with the administration of the subsequent dose of the ActRIIB ligand trap or taken about 3 weeks, 6 weeks, 9 weeks, 12 weeks, 15 weeks, 18 weeks, 21 weeks, or 24 weeks after the administration of the initial dose of the ActRIIB ligand trap. In some embodiments, the second measurement of hemoglobin (Hgb) level is taken concurrently with the administration of the subsequent dose of the ActRIIB ligand trap. In some embodiments, the second measurement of hemoglobin (Hgb) level is taken about 3 weeks, 6 weeks, 9 weeks, 12 weeks, 15 weeks, 18 weeks, 21 weeks, or 24 weeks after the administration of the initial dose of the ActRIIB ligand trap. In some embodiments, the second measurement is taken about 6 weeks after the administration of the initial dose of the ActRIIB ligand trap.

In some embodiments, the initial dose of the ActRIIB ligand trap is 0.3 mg/kg, 0.33 mg/kg, 0.45 mg/kg, 0.6 mg/kg, 0.8 mg/kg, 1.0 mg/kg, 1.33 mg/kg, 1.75 mg/kg, or 2.0 mg/kg. In some embodiments, the initial dose of the ActRIIB ligand trap is 0.30 mg/kg. In some embodiments, the initial dose of the ActRIIB ligand trap is 0.33 mg/kg. In some embodiments, the initial dose of the ActRIIB ligand trap is 0.45 mg/kg. In some embodiments, the initial dose of the ActRIIB ligand trap is 0.6 mg/kg. In some embodiments, the initial dose of the ActRIIB ligand trap is 0.8 mg/kg. In some embodiments, the initial dose of the ActRIIB ligand trap is 1.0 mg/kg. In preferred embodiments, the initial dose of the ActRIIB ligand trap is 1.33 mg/kg. In some embodiments, the initial dose of the ActRIIB ligand trap is 1.75 mg/kg. In some embodiments, the initial dose of the ActRIIB ligand trap is 2.0 mg/kg.

In some embodiments, the subsequent dose of the ActRIIB ligand trap is 0.30 mg/kg, 0.33 mg/kg, 0.45 mg/kg, 0.6 mg/kg, 0.8 mg/kg, 1.0 mg/kg, 1.33 mg/kg, 1.75 mg/kg, or 2.0 mg/kg. In some embodiments, the subsequent dose of the ActRIIB ligand trap is 0.6 mg/kg, 0.8 mg/kg, 1.0 mg/kg, 1.33 mg/kg, or 1.75 mg/kg. In some embodiments, the subsequent dose of the ActRIIB ligand trap is 0.30 mg/kg. In some embodiments, the subsequent dose of the ActRIIB ligand trap is 0.33 mg/kg. In some embodiments, the subsequent dose of the ActRIIB ligand trap is 0.45 mg/kg. In some embodiments, the subsequent dose of the ActRIIB ligand trap is 0.6 mg/kg. In some embodiments, the subsequent dose of the ActRIIB ligand trap is 0.8 mg/kg. In some embodiments, the subsequent dose of the ActRIIB ligand trap is 1.0 mg/kg. In some embodiments, the subsequent dose of the ActRIIB ligand trap is 1.33 mg/kg. In some embodiments, the subsequent dose of the ActRIIB ligand trap is 1.75 mg/kg. In some embodiments, the subsequent dose of the ActRIIB ligand trap is 2.0 mg/kg.

In certain embodiments, the subsequent dose of the ActRIIB ligand trap is about 0.5 mg, about 0.5 mg, about 0.6 mg, about 0.7 mg, about 0.8 mg, about 0.9 mg, about 1 mg, about 1.1 mg, about 1.2 mg, about 1.3 mg, about 1.4 mg, about 1.5 mg, about 1.6 mg, about 1.7 mg, about 1.8 mg, about 1.9 mg, about 2.0 mg, about 2.1 mg, about 2.2 mg, about 2.3 mg, about 2.4 mg, about 2.5 mg, about 2.5 mg, about 2.6 mg, about 2.7 mg, about 2.8 mg, about 2.9 mg, about 3.0 mg, about 3.5 mg, about 4.0 mg, about 4.5 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, or about 35 mg greater than the initial dose of the ActRIIB ligand trap. In certain embodiments, the subsequent dose of the ActRIIB ligand trap is about 0.05 mg/kg, about 0.06 mg/kg, about 0.07 mg/kg, about 0.08 mg/kg, about 0.09 mg/kg, about 0.1 mg/kg, about 0.11 mg/kg, about 0.12 mg/kg, about 0.13 mg/kg, about 0.14 mg/kg, about 0.15 mg/kg, about 0.16 mg/kg, about 0.17 mg/kg, about 0.18 mg/kg, about 0.19 mg/kg, about 0.20 mg/kg, about 0.21 mg/kg, about 0.22 mg/kg, about 0.23 mg/kg, about 0.24 mg/kg, about 0.25 mg/kg, about 0.26 mg/kg, about 0.27 mg/kg, about 0.28 mg/kg, about 0.29 mg/kg, about 0.3 mg/kg, about 0.35 mg/kg, about 0.4 mg/kg, about 0.45 mg/kg, or about 0.5 mg/kg greater than the initial dose of the ActRIIB ligand trap.

In certain embodiments, the subsequent dose is about 0.5 mg, about 0.5 mg, about 0.6 mg, about 0.7 mg, about 0.8 mg, about 0.9 mg, about 1 mg, about 1.1 mg, about 1.2 mg, about 1.3 mg, about 1.4 mg, about 1.5 mg, about 1.6 mg, about 1.7 mg, about 1.8 mg, about 1.9 mg, about 2.0 mg, about 2.1 mg, about 2.2 mg, about 2.3 mg, about 2.4 mg, about 2.5 mg, about 2.5 mg, about 2.6 mg, about 2.7 mg, about 2.8 mg, about 2.9 mg, about 3.0 mg, about 3.5 mg, about 4.0 mg, about 4.5 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, or about 35 mg greater than the initial dose, or about 0.05 mg/kg, about 0.06 mg/kg, about 0.07 mg/kg, about 0.08 mg/kg, about 0.09 mg/kg, about 0.1 mg/kg, about 0.11 mg/kg, about 0.12 mg/kg, about 0.13 mg/kg, about 0.14 mg/kg, about 0.15 mg/kg, about 0.16 mg/kg, about 0.17 mg/kg, about 0.18 mg/kg, about 0.19 mg/kg, about 0.20 mg/kg, about 0.21 mg/kg, about 0.22 mg/kg, about 0.23 mg/kg, about 0.24 mg/kg, about 0.25 mg/kg, about 0.26 mg/kg, about 0.27 mg/kg, about 0.28 mg/kg, about 0.29 mg/kg, about 0.3 mg/kg, about 0.35 mg/kg, about 0.4 mg/kg, about 0.45 mg/kg, or about 0.5 mg/kg less than the initial dose.

In some embodiments, the subsequent dose of the ActRIIB ligand trap is the same as the initial dose of the ActRIIB ligand trap.

In some embodiments, when the second measurement of Hgb level is 0.5 g/L or more, 1.0 g/L or more, 1.5 g/L or more, 2.0 g/L or more, or 2.5 g/L or more higher than the first measurement of Hgb level, the subsequent dose of the ActRIIB ligand trap is lower than the initial dose of the ActRIIB ligand trap. In some embodiments, when the second measurement of Hgb level is 2.0 g/L or more higher than the first measurement of Hgb level, the subsequent dose of the ActRIIB ligand trap is lower than the initial dose of the ActRIIB ligand trap.

In some embodiments, when the second measurement of Hgb level is 0.5 g/L or more, 1.0 g/L or more, 1.5 g/L or more, 2.0 g/L or more, or 2.5 g/L or more higher than the first measurement of Hgb level, the dose of the ActRIIB ligand trap to be administered is reduced. In some embodiments, when the second measurement of Hgb level is 2.0 g/L or more higher than the first measurement of Hgb level, the dose of the ActRIIB ligand trap to be administered is reduced.

In some embodiment, when the subject has one or more RBC transfusion during the first period of time, or the second measurement of Hgb level is between 0 to about 1 g/dL higher than the first measurement of Hgb level, or the first measurement of Hgb level decreases 1 g/dL or more in a transfusion-free period of approximately 6 weeks, the subsequent dose of the ActRIIB ligand trap is higher than the initial dose of the ActRIIB ligand trap. In some embodiment, when the subject has one or more RBC transfusion during the first period of time, the subsequent dose of the ActRIIB ligand trap is higher than the initial dose of the ActRIIB ligand trap. In some embodiment, when the second measurement of Hgb level is between 0 to about 1 g/dL higher than the first measurement of Hgb level, the subsequent dose of the ActRIIB ligand trap is higher than the initial dose of the ActRIIB ligand trap. In some embodiment, when the first measurement of Hgb level decreases 1 g/dL or more in a transfusion-free period of approximately 6 weeks, the subsequent dose of the ActRIIB ligand trap is higher than the initial dose of the ActRIIB ligand trap.

In some embodiments, when the subject has a grade 4 thrombocytopenia or Grade 3 thrombocytopenia with active bleeding, the administration of the ActRIIB ligand trap is delayed. In some embodiments, the administration of the ActRIIB ligand trap is delayed for 1, 2, 3, 4, 5, 6, 7, 8, 9, 10, 11, or 12 weeks. In one embodiment, the administration of the ActRIIB ligand trap is delayed for 1 cycle. In another embodiment, the administration of the ActRIIB ligand trap is delayed for 2 cycles. In yet another embodiment, the administration of the ActRIIB ligand trap is delayed for 3 cycles. In yet another embodiment, the administration of the ActRIIB ligand trap is delayed for 4 cycles. In yet another embodiment, the administration of the ActRIIB ligand trap is delayed for up to 3 weeks. In yet another embodiment, the administration of the ActRIIB ligand trap is delayed for up to 6 weeks. In yet another embodiment, the administration of the ActRIIB ligand trap is delayed for up to 9 weeks. In yet another embodiment, the administration of the ActRIIB ligand trap is delayed for up to 12 weeks. In some embodiments, white blood cells, neutrophils and platelet counts are measured weekly during the delay of the administration of the ActRIIB ligand trap.

In some embodiments, when the subject has a grade 4 neutropenia or grade 4 leukopenia (worsening by 2 grades), the administration of the ActRIIB ligand trap is delayed. In some embodiments, the administration of the ActRIIB ligand trap is delayed for 1, 2, 3, 4, 5, 6, 7, 8, 9, 10, 11, or 12 weeks. In one embodiment, the administration of the ActRIIB ligand trap is delayed for 1 cycle. In another embodiment, the administration of the ActRIIB ligand trap is delayed for 2 cycles. In yet another embodiment, the administration of the ActRIIB ligand trap is delayed for 3 cycles. In yet another embodiment, the administration of the ActRIIB ligand trap is delayed for 4 cycles. In yet another embodiment, the administration of the ActRIIB ligand trap is delayed for up to 3 weeks. In yet another embodiment, the administration of the ActRIIB ligand trap is delayed for up to 6 weeks. In yet another embodiment, the administration of the ActRIIB ligand trap is delayed for up to 9 weeks. In yet another embodiment, the administration of the ActRIIB ligand trap is delayed for up to 12 weeks. In some embodiments, white blood count (WBC), neutrophils and platelet counts are measured weekly during the delay of the administration of the ActRIIB ligand trap.

In some embodiments, when the subject has a White blood count (WBC) of 120×10⁹/L or more, or 30×10⁹/L or more and more than three times of the baseline WBC, the administration of the ActRIIB ligand trap is delayed. In some embodiments, the administration of the ActRIIB ligand trap is delayed for 1, 2, 3, 4, 5, 6, 7, 8, 9, 10, 11, or 12 weeks. In one embodiment, the administration of the ActRIIB ligand trap is delayed for 1 week. In another embodiment, the administration of the ActRIIB ligand trap is delayed for 2 weeks. In yet another embodiment, the administration of the ActRIIB ligand trap is delayed for 3 weeks. In yet another embodiment, the administration of the ActRIIB ligand trap is delayed for 4 weeks. In some embodiments, the administration of the ActRIIB ligand trap is delayed and the WBC of the subject is measured weekly. In some embodiments, the administration of the ActRIIB ligand trap is delayed until the WBC of the subject becomes less than 120×10⁹/L, less than 30×10⁹/L, or less than three times of the baseline WBC.

### 5.5.2 Dose Adjustment or Delay of Fedratinib

In some embodiments, the dose of fedratinib administered to the subject is reduced when the subject has an adverse event. In some embodiments, the dose of fedratinib administered to the subject is reduced when the subject has a hematological, hepatic, non-hematological, or gastrointestinal adverse event graded as a Grade 1, 2, 3, 4, or 5 event according to the National Cancer Institute (NCI) Common Terminology Criteria for Adverse Events (CTCAE). In some embodiment, the dose of fedratinib administered to the subject is reduced to 300 mg/day, 200 mg/day, or 100 mg/day. In one embodiment, the dose of fedratinib administered to the subject is reduced to 300 mg/day. In another embodiment, the dose of fedratinib administered to the subject is reduced to 200 mg/day. In yet another embodiment, the dose of fedratinib administered to the subject is reduced to 100 mg/day. In yet another embodiment, the dose of fedratinib administered to the subject is reduced to 0 mg/day. In yet another embodiment, the administration of fedratinib is discontinued.

In some embodiments, when the subject has a grade 4 thrombocytopenia or Grade 3 thrombocytopenia with active bleeding, the administration of fedratinib is delayed. In some embodiments, the administration of fedratinib is delayed for 1, 2, 3, 4, 5, 6, 7, 8, 9, 10, 11, or 12 weeks. In one embodiment, the administration of fedratinib is delayed for 1 week. In another embodiment, the administration of fedratinib is delayed for 2 weeks. In yet another embodiment, the administration of fedratinib is delayed for 3 weeks. In yet another embodiment, the administration of fedratinib is delayed for 4 weeks. In yet another embodiment, the administration of fedratinib is delayed for up to 3 weeks. In yet another embodiment, the administration of fedratinib is delayed for up to 4 weeks. In yet another embodiment, the administration of fedratinib is delayed for up to 3 weeks until thrombocytopenia in the subject becomes Grade 3 or less. In yet another embodiment, the administration of fedratinib is delayed for up to 4 weeks until thrombocytopenia in the subject becomes Grade 3 or less. In yet another embodiment, the administration of fedratinib is delayed for up to 3 weeks until thrombocytopenia in the subject becomes Grade 3 or less without bleeding. In yet another embodiment, the administration of fedratinib is delayed for up to 4 weeks until thrombocytopenia in the subject becomes Grade 3 or less without bleeding. In some embodiments, when fedratinib is administered after delay, fedratinib is administered at a subsequent dose of fedratinib. In some embodiments, the subsequent dose of fedratinib is the same as the dose of fedratinib prior to the delay. In some embodiments, the subsequent dose of fedratinib is 400 mg/day. In some embodiments, the subsequent dose of fedratinib is 100 mg/day lower than the dose of fedratinib prior to the delay. In some embodiments, the dose of fedratinib administered to the subject is reduced to 300 mg/day, 200 mg/day, or 100 mg/day. In some embodiments, the subsequent dose of fedratinib is 300 mg/day, 200 mg/day, or 100 mg/day.

In some embodiments, when the subject has a grade 4 neutropenia, the administration of fedratinib is delayed. In some embodiments, the administration of fedratinib is delayed for 1, 2, 3, 4, 5, 6, 7, 8, 9, 10, 11, or 12 weeks. In one embodiment, the administration of fedratinib is delayed for 1 week. In another embodiment, the administration of fedratinib is delayed for 2 weeks. In yet another embodiment, the administration of fedratinib is delayed for 3 weeks. In yet another embodiment, the administration of fedratinib is delayed for 4 weeks. In yet another embodiment, the administration of fedratinib is delayed for up to 3 weeks. In yet another embodiment, the administration of fedratinib is delayed for up to 4 weeks. In yet another embodiment, the administration of fedratinib is delayed for up to 3 weeks until neutropenia in the subject becomes Grade 2 or less. In yet another embodiment, the administration of fedratinib is delayed for up to 4 weeks until neutropenia in the subject becomes Grade 2 or less. In some embodiments, when fedratinib is administered after delay, fedratinib is administered at a subsequent dose of fedratinib. In some embodiments, the subsequent dose of fedratinib is the same as the dose of fedratinib prior to the delay. In some embodiments, the subsequent dose of fedratinib is 400 mg/day. In some embodiments, the subsequent dose of fedratinib is 100 mg/day lower than the dose of fedratinib prior to the delay. In some embodiments, the dose of fedratinib administered to the subject is reduced to 300 mg/day, 200 mg/day, or 100 mg/day. In some embodiments, the subsequent dose of fedratinib is 300 mg/day, 200 mg/day, or 100 mg/day.

In some embodiments, the treatment efficacy of the methods described herein are measured using modified Hematological Improvement—Erythroid Response (HI-E). In some embodiments, the HI-E are defined using International Working Group (IWG) Response Criteria for MDS (MDS-IWG criteria) over any consecutive ≥84-day period (12 weeks). MDS-IWG criteria are disclosed, for example, in Cheson et al., 2006, Blood. 108:419-425, the disclosure of which is incorporated herein by reference in its entirety.

In some embodiments, the methods described herein increase the hemoglobin (Hgb) level in the subject by at least 0.5 g/dL, at least 1.0 g/dL, at least 1.5 g/dL, at least 2.0 g/dL, or at least 2.5 g/dL. In some embodiment, the methods described herein increase the hemoglobin (Hgb) level in the subject by at least 0.5 g/dL. In some embodiment, the methods described herein increase the hemoglobin (Hgb) level in the subject by at least 1.0/dL. In some embodiment, the methods described herein increase the hemoglobin (Hgb) level in the subject by at least 1.5 g/dL. In some embodiment, the methods described herein increase the hemoglobin (Hgb) level in the subject by at least 2.0 g/dL. In some embodiment, the methods described herein increase the hemoglobin (Hgb) level in the subject by at least 2.5 g/dL. In some embodiments, the Hgb level increase is compared to baseline. In preferred embodiments, the baseline is the Hgb level in the subject prior to administration of fedratinib and/or the ActRIIB ligand trap to the subject. In some embodiments, the baseline is the Hgb level in a reference population.

In some embodiments, the methods described herein reduce RBC transfusion burden of the subject by at least 10%, at least 20%, at least 30%, at least 40%, at least 50%, at least 60%, at least 70%, at least 80%, at least 90%, or at least 100%. In some embodiments, the methods described herein reduce RBC transfusion burden of the subject by at least 40%. In some embodiments, the methods described herein reduce RBC transfusion burden of the subject by at least 50%. In some embodiments, the methods described herein reduce RBC transfusion burden of the subject by at least 60%. In some embodiments, the methods described herein reduce RBC transfusion burden of the subject by at least 70%.

In some embodiments, the methods described herein reduce at least 2, at least 3, at least 4, at least 5, at least 6, at least 7, at least 8, at least 9, or at least 10 units of red blood cell (RBC) transfusions received by the subject within a period of 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days. In some embodiments, the methods described herein reduce at least 4 units of red blood cell (RBC) transfusions received by the subject within a period of 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days. In some embodiments, the methods described herein reduce at least 8 units of red blood cell (RBC) transfusions received by the subject within a period of 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days. In some embodiments, the methods described herein reduce at least 4 units of red blood cell (RBC) transfusions received by the subject within a period of 56 days. In some embodiments, the methods described herein reduce at least 4 units of red blood cell (RBC) transfusions received by the subject within a period of 63 days. In some embodiments, the methods described herein reduce at least 4 units of red blood cell (RBC) transfusions received by the subject within a period of 70 days. In some embodiments, the methods described herein reduce at least 4 units of red blood cell (RBC) transfusions received by the subject within a period of 77 days. In some embodiments, the methods described herein reduce at least 4 units of red blood cell (RBC) transfusions received by the subject within a period of 84 days.

In some embodiments, the subject becomes RBC transfusion free over a consecutive period of 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 98, or 105 days. In some embodiments, the subject becomes RBC transfusion free over a consecutive period of 84 days. In some embodiments, the subject becomes RBC transfusion free over a consecutive period of 91 days. In some embodiments, the subject becomes RBC transfusion free over a consecutive period of 105 days. In some embodiments, the subject becomes non-transfusion dependent over a consecutive period of 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 98, or 105 days. In some embodiments, the subject becomes non-transfusion dependent over a consecutive period of 84 days. In some embodiments, the subject becomes RBC transfusion free over a consecutive period of 91 days. In some embodiments, the subject becomes non-transfusion dependent over a consecutive period of 105 days.

In some embodiments, the subject becomes RBC transfusion free over a consecutive period of at least 6 weeks, 7 weeks, 8 weeks, 9 weeks, 10 weeks, 11 weeks, 12 weeks, 13 weeks, 14 weeks, or 15 weeks. In some embodiments, the subject becomes RBC transfusion free over a consecutive period of at least 8 weeks. In some embodiments, the subject becomes RBC transfusion free over a consecutive period of at least 10 weeks. In some embodiments, the subject becomes RBC transfusion free over a consecutive period of at least 15 weeks. In some embodiments, the subject becomes non-transfusion dependent over a consecutive period of at least 6 weeks, 7 weeks, 8 weeks, 9 weeks, 10 weeks, 11 weeks, 12 weeks, 13 weeks, 14 weeks, or 15 weeks. In some embodiments, the subject becomes non-transfusion dependent over a consecutive period of at least 8 weeks. In some embodiments, the subject becomes non-transfusion dependent over a consecutive period of at least 10 weeks. In some embodiments, the subject becomes non-transfusion dependent over a consecutive period of at least 15 weeks.

In some embodiments, the methods described herein increase the hemoglobin (Hgb) level in the subject by at least 1.5 g/dL or reduce at least 4 units of red blood cell (RBC) transfusions received by the subject within a period of 56 days compared with baseline, wherein the subject is red blood cell transfusion dependent or non-transfusion dependent.

In some embodiments, the methods described herein reduce RBC transfusion burden of the subject by at least 50% and reduce at least 4 units of red blood cell (RBC) transfusions received by the subject within a period of 84 days from baseline, wherein the subject is red blood cell transfusion dependent.

In some embodiments, the methods described herein increase the hemoglobin (Hgb) level in the subject by at least 1.5 g/dL compared with baseline and the subject becomes RBC transfusion free over a consecutive period of 84 days, wherein the subject is non-transfusion dependent. In specific embodiments, the methods described herein increase the hemoglobin (Hgb) level in the subject by at least 1.5 g/dL compared with baseline over a consecutive period of 84 days without an RBC transfusion (transfusion free), wherein the subject is non-transfusion dependent.

In some embodiments, the subject becomes RBC transfusion free over a consecutive period of 84 days, wherein the subject is red blood cell transfusion dependent or non-transfusion dependent.

In some embodiments, the methods described herein increase hemoglobin (HGB) levels in the subject to levels equal to or about 1%, 2%, 3%, 4%, 5%, 6%, 7%, 8%, 9%, 10%, 20%, 25%, 30%, 40%, 50%, 60%, 70%, 75%, 80%, 90%, 100%, 200%, or 500% greater than HGB levels in the subject prior to said treating. In some embodiments, the methods described herein increase hemoglobin (HGB) levels in the subject to levels equal to or about 1%, 2%, 3%, 4%, 5%, 6%, 7%, 8%, 9%, 10%, 20%, 25%, 30%, 40%, 50%, 60%, 70%, 75%, 80%, 90%, 100%, 200%, or 500% greater than HGB levels in the subject prior to administration of fedratinib and/or the ActRIIB ligand trap to the subject.

In some embodiments, the methods described herein increase hematocrit (HCT) levels in the subject to levels equal to or about 1%, 2%, 3%, 4%, 5%, 6%, 7%, 8%, 9%, 10%, 20%, 25%, 30%, 40%, 50%, 60%, 70%, 75%, 80%, 90%, 100%, 200%, or 500% greater than HCT levels in the subject prior to said treating. In some embodiments, the methods described herein increase hematocrit (HCT) levels in the subject to levels equal to or about 1%, 2%, 3%, 4%, 5%, 6%, 7%, 8%, 9%, 10%, 20%, 25%, 30%, 40%, 50%, 60%, 70%, 75%, 80%, 90%, 100%, 200%, or 500% greater than HCT levels in the subject prior to prior to administration of fedratinib and/or the ActRIIB ligand trap to the subject.

In some embodiments, the methods described herein reduce mean corpuscular volume (MCV) levels in the subject to levels equal to or about 1%, 2%, 3%, 4%, 5%, 6%, 7%, 8%, 9%, 10%, 20%, 25%, 30%, 40%, 50%, 60%, 70%, 75%, 80%, 90%, or 100%, less than MCV levels in the subject prior to said treating. In some embodiments, the methods described herein reduce mean corpuscular volume (MCV) levels in the subject to levels equal to or about 1%, 2%, 3%, 4%, 5%, 6%, 7%, 8%, 9%, 10%, 20%, 25%, 30%, 40%, 50%, 60%, 70%, 75%, 80%, 90%, or 100%, less than MCV levels in the subject prior to administration of fedratinib and/or the ActRIIB ligand trap to the subject.

In some embodiments, the methods described herein increase cellular hemoglobin concentration (CHC) levels in the subject to levels equal to or about 1%, 2%, 3%, 4%, 5%, 6%, 7%, 8%, 9% 10%, 20%, 25%, 30%, 40%, 50%, 60%, 70%, 75%, 80%, 90%, 100%, 200%, or 500% greater than CHC levels in the subject prior to said treating. In some embodiments, the methods described herein increase cellular hemoglobin concentration (CHC) levels in the subject to levels equal to or about 1%, 2%, 3%, 4%, 5%, 6%, 7% 8%, 9% 10%, 20%, 25%, 30%, 40%, 50%, 60%, 70%, 75%, 80%, 90%, 100%, 200%, or 500% greater than CHC levels in the subject prior to administration of fedratinib and/or the ActRIIB ligand trap to the subject.

In some embodiments, the methods described herein reduce red blood cell distribution width (RDW) levels in the subject to levels equal to or about 1%, 2%, 3%, 4%, 5%, 6%, 7%, 8%, 9%10%, 20%, 25%, 30%, 40%, 50%, 60%, 70%, 75%, 80%, 90%, or 100%, less than the RDW levels in the subject prior to said treating. In some embodiments, the methods described herein reduce red blood cell distribution width (RDW) levels in the subject to levels equal to or about 1%, 2%, 3%, 4%, 5%, 6%, 7%, 8%, 9%, 10%, 20%, 25%, 30%, 40%, 50%, 60%, 70%, 75%, 80%, 90%, or 100%, less than the RDW levels in the subject prior to administration of fedratinib and/or the ActRIIB ligand trap to the subject.

In certain embodiments, anemia response is measured. In some embodiments anemia response is measured as it relates to hemoglobin (Hgb) increase, e.g., proportion of subjects achieving ≥1.5 g/dL hemoglobin increase from baseline over any consecutive 84-day period without an RBC transfusion. In some embodiments anemia response is measured as it relates to increased red blood cell (RBC)-transfusion independence, e.g., proportion of subjects who become RBC-transfusion free over any consecutive 84-day period. In some embodiments anemia response is measured as it relates to time to anemia response, e.g., time from first luspatercept dose to first onset of anemia response. In some embodiments anemia response is measured as it relates to duration of anemia response, e.g., the maximum duration of anemia response in subjects. In some embodiments anemia response is measured as it relates to frequency of RBC transfusions, e.g., the mean number of RBC units transfused per subject per four weeks. In some embodiments anemia response is measured as it relates to frequency of RBC transfusion dependence, e.g., proportion of RBC transfusion dependent subjects who reduce their transfusion burden by ≥50% from baseline over any consecutive 84-day period. In some embodiments anemia response is measured as it relates to proportion of subjects who achieve a reduction in fatigue symptom as recorded and assessed via the modified Myeloproliferative Neoplasm Symptom Assessment Form (MPN-SAF), (see Emanual et al., “Myeloproliferative Neoplasm (MPN) Symptom Assessment Form Total Symptom Score: Prospective International Assessment of an Abbreviated Symptom Burden Scoring System Among Patients With MPNs,” J. Clin. Oncol. 30(33):4098-4103 (2012)), e.g., measuring fatigue, night sweats, itchiness, abdominal discomfort, pain under the ribs on the left side, early satiety, and bone pain (e.g., in subjects who achieve ≥50% reduction in fatigue symptom as measured by the MPN-SAF. In some embodiments anemia response is measured as it relates to the proportion of subjects who achieve ≥50% reduction in total symptom score (TSS) as recorded and assessed via the modified Myeloproliferative Neoplasm Symptom Assessment Form (MPN-SAF), (see Emanual et al., “Myeloproliferative Neoplasm (MPN) Symptom Assessment Form Total Symptom Score: Prospective International Assessment of an Abbreviated Symptom Burden Scoring System Among Patients With MPNs,” J. Clin. Oncol. 30(33):4098-4103 (2012), e.g., measuring fatigue, night sweats, itchiness, abdominal discomfort, pain under the ribs on the left side, early satiety, and bone pain. In some embodiments anemia response is measured as it relates to Health-related quality of life (HRQoL) measures, e.g., measure mean changes in HRQoL questionnaire domain scores compared to baseline scores. In some embodiments anemia response is measured as it relates to EQ-5D-5L questionnaires scores (EuroQol, Rotterdam, The Netherlands), e.g., measuring mean changes in EQ-5D-5L questionnaire domain scores compared to baseline scores. In some embodiments anemia response is measured as it relates to adverse events (AEs) e.g., the type, frequency, and severity, of adverse events (. In some embodiments anemia response is measured using antidrug antibodies (ADA) (e.g., frequency of antidrug antibodies and effects on efficacy, safety, or pharmacokinetics in subjects). In some embodiments anemia response is measured using pharmacokinetic parameters, e.g., plasma concentration-time curve, area under the curve (AUC), Cmax.

Also provided herein are methods for treating anemia in a subject in need thereof, wherein the method comprises administering to the subject a dose of 0.33 mg/kg, 0.45 mg/kg, 0.6 mg/kg, 0.8 mg/kg, 1 mg/kg, 1.33 mg/kg, 1.75 mg/kg, or 2.0 mg/kg of an ActRIIB ligand trap (such as the ActRIIB ligand traps described in Section 5.4) comprising an amino acid sequence consisting of the amino acid sequence of SEQ ID NO:11, wherein the ActRIIB ligand trap is administered to the subject subcutaneously once every 21 days; and administering to the subject fedratinib or a pharmaceutically acceptable salt or hydrate thereof (as described in Section 5.3). In certain embodiments, the ActRIIB ligand trap and fedratinib or pharmaceutically acceptable salt or hydrate thereof are concomitantly administered. In certain embodiments, the co-administration of the ActRIIB ligand trap and fedratinib or pharmaceutically acceptable salt and/or hydrate thereof is pharmaceutically effective to treat anemia.

Also provided herein are methods for treating anemia in a subject in need thereof, wherein the method comprises administering to the subject a dose of 0.33 mg/kg, 0.45 mg/kg, 0.6 mg/kg, 0.8 mg/kg, 1 mg/kg, 1.33 mg/kg, 1.75 mg/kg, or 2.0 mg/kg of an ActRIIB ligand trap (such as the ActRIIB ligand traps described in Section 5.4) comprising an amino acid sequence of SEQ ID NO:11, wherein the ActRIIB ligand trap is administered to the subject subcutaneously once every 21 days; and administering to the subject fedratinib or a pharmaceutically acceptable salt and/or hydrate thereof (as described in Section 5.3). In certain embodiments, the ActRIIB ligand trap and fedratinib or pharmaceutically acceptable salt or hydrate thereof are concomitantly administered. In certain embodiments, the co-administration of the ActRIIB ligand trap and fedratinib or pharmaceutically acceptable salt or hydrate thereof is pharmaceutically effective to treat anemia.

Also provided herein are methods for treating anemia in a subject in need thereof, wherein the method comprises administering to the subject an ActRIIB ligand trap (such as the ActRIIB ligand traps described in Section 5.4), and administering to the subject fedratinib or a pharmaceutically acceptable salt and/or hydrate thereof (as described in Section 5.3), wherein the method increases the hemoglobin (Hgb) level in the subject by at least 0.5 g/dL, at least 1.0 g/dL, at least 1.5 g/dL, at least 2.0 g/dL, or at least 2.5 g/dL. In certain embodiments, the ActRIIB ligand trap and fedratinib or pharmaceutically acceptable salt or hydrate thereof are concomitantly administered. In certain embodiments, the co-administration of the ActRIIB ligand trap and fedratinib or pharmaceutically acceptable salt or hydrate thereof is pharmaceutically effective to treat anemia.

Also provided herein are methods for treating anemia in a subject in need thereof, wherein the method comprises administering to the subject an ActRIIB ligand trap (such as the ActRIIB ligand traps described in Section 5.4), and administering to the subject fedratinib or a pharmaceutically acceptable salt and/or hydrate thereof (as described in Section 5.3), wherein the method reduces RBC transfusion burden of the subject by at least 10%, at least 20%, at least 30%, at least 40%, at least 50%, at least 60%, at least 70%, at least 80%, at least 90%, or at least 100%. In certain embodiments, the ActRIIB ligand trap and fedratinib or pharmaceutically acceptable salt or hydrate thereof are concomitantly administered. In certain embodiments, the co-administration of the ActRIIB ligand trap and fedratinib or pharmaceutically acceptable salt or hydrate thereof is pharmaceutically effective to treat anemia.

Also provided herein are methods for treating anemia in a subject in need thereof, wherein the method comprises administering to the subject an ActRIIB ligand trap (such as the ActRIIB ligand traps described in Section 5.4), and administering to the subject fedratinib or a pharmaceutically acceptable salt and/or hydrate thereof (as described in Section 5.3), wherein the method reduces at least 2, at least 3, at least 4, at least 5, at least 6, at least 7, at least 8, at least 9, or at least 10 units of red blood cell (RBC) transfusions received by the subject within a period of 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days. In certain embodiments, the ActRIIB ligand trap and fedratinib or pharmaceutically acceptable salt or hydrate thereof are concomitantly administered. In certain embodiments, the co-administration of the ActRIIB ligand trap and fedratinib or pharmaceutically acceptable salt or hydrate thereof is pharmaceutically effective to treat anemia.

Also provided herein are methods for treating anemia in a subject in need thereof, wherein the method comprises administering to the subject an ActRIIB ligand trap (such as the ActRIIB ligand traps described in Section 5.4), and administering to the subject fedratinib or a pharmaceutically acceptable salt and/or hydrate thereof (as described in Section 5.3), wherein the subject becomes RBC transfusion free or non-transfusion dependent over a consecutive period of 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 98, or 105 days.

### 5.6 Patient Population

The subjects treated in accordance with the methods described herein can be any mammals such as rodents and primates, and in a preferred embodiment, human. In certain embodiments, the subject is a human. In certain embodiments, the methods described herein can be used to treat anemia in any mammals, such as rodents and primates, and in a preferred embodiment, in human.

In certain embodiments, the subject treated in accordance with the methods described here can be of any age. In certain embodiments, the subject treated in accordance with the methods described herein is less than 18 years old. In a specific embodiment, the subject treated in accordance with the methods described herein is less than 13 years old. In another specific embodiment, the subject treated in accordance with the methods described herein is less than 12, less than 11, less than 10, less than 9, less than 8, less than 7, less than 6, or less than 5 years old. In another specific embodiment, the subject treated in accordance with the methods described herein is 1-3 years old, 3-5 years old, 5-7 years old, 7-9 years old, 9-11 years old, 11-13 years old, 13-15 years old, 15-20 years old, 20-25 years old, 25-30 years old, or greater than 30 years old. In another specific embodiment, the subject treated in accordance with the methods described herein is 30-35 years old, 35-40 years old, 40-45 years old, 45-50 years old, 50-55 years old, 55-60 years old, or greater than 60 years old. In another specific embodiment, the subject treated in accordance with the methods described herein is 60-65 years old, 65-70 years old, 70-75 years old, 75-80 years old, or greater than 80 years old.

In certain embodiments, the subject requires regular, lifelong red blood cell transfusions. In certain embodiments, the subject has a high transfusion burden. In certain embodiments, high transfusion burden is 12 or more red blood cell units over 24 weeks prior to treatment according to the methods provided herein. In certain embodiments, the subject has a low transfusion burden. In certain embodiments, low transfusion burden is 7-12 red blood cell units over 24 weeks prior to treatment according to the methods provided herein.

In some embodiments, the subject treated according to the methods provided herein is a subject diagnosed with myelofibrosis. In some embodiments, the subject is a subject diagnosed with myeloproliferative neoplasms (MPN)-associated myelofibrosis. In certain embodiments, the myelofibrosis is a myeloproliferative neoplasms (MPN)-associated myelofibrosis. In certain embodiments, the myelofibrosis is intermediate or high-risk myeloproliferative neoplasms (MPN)-associated myelofibrosis. In certain embodiments, the myelofibrosis is Primary Myelofibrosis (PMF), Post-Polycythemia Vera Myelofibrosis (post-PV MF), or Post-Essential Thrombocythemia Myelofibrosis (post-ET MF). In certain embodiments, the myelofibrosis is intermediate or high-risk Primary Myelofibrosis (PMF), Post-Polycythemia Vera Myelofibrosis (post-PV MF), or Post-Essential Thrombocythemia Myelofibrosis (post-ET MF). In certain embodiments, the myelofibrosis is intermediate or high-risk primary myelofibrosis (PMF). In certain embodiments, the myelofibrosis is intermediate or high-risk post-polycythemia vera myelofibrosis (post-PV MF). In certain embodiments, the myelofibrosis is intermediate or high-risk post-essential thrombocythemia myelofibrosis (post-ET MF).

In certain embodiments, the subject treated according to the methods provided herein has myeloproliferative neoplasm-associated myelofibrosis. In certain embodiments, the myeloproliferative neoplasm-associated myelofibrosis is primary myelofibrosis. In some embodiments, the myeloproliferative neoplasm-associated myelofibrosis is polycythemia vera myelofibrosis. In more specific embodiments, the polycythemia vera myelofibrosis is associated with, or caused by, a JAK2 mutation, e.g., a JAK2 V617F mutation or JAK2 exon 12 mutation, or a thrombopoietin receptor (MPL) mutation. In specific embodiments, the myeloproliferative neoplasm-associated myelofibrosis is post-polycythemia vera myelofibrosis. In more specific embodiments, the post-polycythemia vera myelofibrosis is associated with, or caused by, a JAK2 mutation, e.g., a JAK2 V617F mutation, or a thrombopoietin receptor (MPL) mutation. In specific embodiments, the myeloproliferative neoplasm-associated myelofibrosis is essential thrombocytopenia or post-essential thrombocythemia myelofibrosis. In more specific embodiments, the essential thrombocytopenia or post-essential thrombocythemia myelofibrosis is associated with, or caused by, a JAK2 mutation, e.g., a JAK2 V617F mutation, or a thrombopoietin receptor (MPL) mutation. In more specific embodiments, the essential thrombocytopenia or post-essential thrombocythemia myelofibrosis includes reticulin fibrosis or trichrome fibrosis.

In certain embodiments, the subject treated according to the methods provided herein has anemia. In certain embodiments, the subject treated according to the methods provided herein has myeloproliferative neoplasm-associated myelofibrosis and anemia. In certain embodiments, the anemia is associated with myelofibrosis. In certain embodiments, the anemia is associated with MPN-associated myelofibrosis. In certain embodiments, the anemia is associated with Primary Myelofibrosis (PMF), Post-Polycythemia Vera Myelofibrosis (post-PV MF), or Post-Essential Thrombocythemia Myelofibrosis (post-ET MF).

In certain embodiments, the methods described herein can be used to treat anemia in a subject, such as, an anemia associated with ineffective erythropoiesis, or thalassemia.

In certain embodiments, the subject treated in accordance with the methods described herein (see Sections 5.5 and 5.9) as beta-thalassemia. In certain embodiments, the beta-thalassemia is transfusion-dependent beta-thalassemia. Transfusion-dependent beta-thalassemia is also known as “Cooley's anemia”. In certain embodiments, the beta-thalassemia is beta-thalassemia major. In certain embodiments, the transfusion-dependent beta-thalassemia is beta-thalassemia major. In certain embodiments, the beta-thalassemia is non-transfusion-dependent beta-thalassemia. In certain embodiments, the beta-thalassemia is beta-thalassemia intermediate. In certain embodiments, the transfusion-dependent beta-thalassemia is non-beta-thalassemia intermediate. In certain embodiments, the subject has HbE/beta thalassemia. In certain embodiments, the subject (i) has beta-thalassemia major; (ii) has severe HbE/beta-thalassemia; and (iii) is transfusion-dependent. In certain embodiments, the subject (i) has beta-thalassemia intermedia; (ii) has mild/moderate HbE/beta-thalassemia; and (iii) is non-transfusion-dependent.

In certain embodiments, the subject treated in accordance with the methods described herein (see Sections 5.5 and 5.9) has transfusion-dependent beta-thalassemia. In certain embodiments, the subject has been diagnosed with transfusion-dependent beta-thalassemia. In certain embodiments, the subject has been diagnosed with beta-thalassemia and hemoglobin E. In certain embodiments, the diagnosis has been confirmed by genetic analysis. In certain embodiments, the transfusion-dependent beta-thalassemia is beta-thalassemia major. In certain embodiments, the transfusion-dependent beta-thalassemia is beta-thalassemia major. In certain embodiments, the subject comprises a genotype comprising homozygosity or compound heterozygosity for a mutant beta globin allele. In certain embodiments, the homozygosity comprises β⁰/β⁰, wherein β⁰ refers to an allele associated with lack of beta globin chain synthesis. In certain embodiments, the homozygosity comprises β⁺/β⁺, wherein β⁺ refers to an allele associated with reduced beta globin chain synthesis. In certain embodiments, the compound heterozygosity comprises β⁰/β⁰, wherein β⁰ refers to an allele associated with lack of beta globin chain synthesis, and wherein β⁺ refers to an allele associated with reduced beta globin chain synthesis. In certain embodiments, the compound heterozygosity comprises β⁰/HbE, wherein β⁰ refers to an allele associated with lack of beta globin chain synthesis, and wherein HbE refers to hemoglobin E. In certain embodiments, the compound heterozygosity comprises β⁺/HbE, wherein β⁺ refers to an allele associated with reduced beta globin chain synthesis, and wherein HbE refers to hemoglobin E. In certain embodiments, the subject has symptomatic thalassemia. In certain embodiments, the subject has co-inherited duplication of the alpha-globin gene. In certain embodiments, the subject has been diagnosed with transfusion-dependent beta-thalassemia. In certain embodiments, the diagnosis has been confirmed by genetic analysis. In certain embodiments, the subject is a human infant subject. In certain embodiments, the subject has hereditary persistence of fetal hemoglobin.

In some embodiments, the subject treated according to the methods provided herein has previously been treated with ruxolitinib. In a specific embodiment, a subject treated according to the methods provided herein has been on a stable dose of ruxolitinib for at least 112 days immediately prior to treatment.

In some embodiments, the subject treated according to the methods provided herein has not previously been treated with ruxolitinib.

In some embodiments, the subject is a human.

In some embodiments, the subject is red blood cell transfusion dependent or non-transfusion dependent. In certain embodiments, the subject is red blood cell transfusion dependent. In certain embodiments, the subject is non-transfusion dependent. In certain embodiments, the subject is red blood cell transfusion-independent.

In some embodiments, as used herein, “transfusion” refers to red blood cell (RBC) transfusion.

In some embodiments, the subject is non-transfusion dependent or red blood cell transfusion-independent if the subject has received less than 4 RBC units in RBC transfusion during a period of time of 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days prior to administration of fedratinib and/or the ActRIIB ligand trap to the subject. In some embodiments, the subject is non-transfusion dependent or RBC transfusion-independent if the subject has received 1, 2, or 3 RBC units in RBC transfusion during a period of time of 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days prior to administration of fedratinib and/or the ActRIIB ligand trap to the subject. In some embodiments, the subject is non-transfusion dependent or RBC transfusion-independent if the subject has received no (0) RBC units during a period of time of 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days prior to administration of fedratinib and/or the ActRIIB ligand trap to the subject. In some embodiments, the subject is non-transfusion dependent or RBC transfusion-independent if the subject has received no (0) RBC transfusion during a period of time of 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days prior to administration of fedratinib and/or the ActRIIB ligand trap to the subject.

In one embodiment, the subject is non-transfusion dependent or RBC transfusion-independent if the subject has received less than 4 RBC units in RBC transfusion during a period of time of 84 days prior to administration of fedratinib and/or the ActRIIB ligand trap to the subject. In one embodiment, the subject is non-transfusion dependent or RBC transfusion-independent if the subject has received 1, 2, or 3 RBC units in RBC transfusion during a period of time of 84 days prior to administration of fedratinib and/or the ActRIIB ligand trap to the subject. In one embodiment, the subject is non-transfusion dependent or RBC transfusion-independent if the subject has received no (0) RBC units in RBC transfusion during a period of time of 84 days prior to administration of fedratinib and/or the ActRIIB ligand trap to the subject. In one embodiment, the subject is non-transfusion dependent or RBC transfusion-independent if the subject has received no (0) RBC transfusion during a period of time of 84 days prior to administration of fedratinib and/or the ActRIIB ligand trap to the subject.

In one embodiment, the non-transfusion dependent or RBC transfusion-independent subject has received less than 4 RBC units in RBC transfusion during a period of time of 84 days prior to administration of fedratinib and/or the ActRIIB ligand trap to the subject, and the subject has a hemoglobin (Hgb) level of less than 9.5 g/dL. In another embodiment, the non-transfusion dependent or RBC transfusion-independent subject has received no (0) RBC units in RBC transfusion during a period of time of 84 days prior to administration of fedratinib and/or the ActRIIB ligand trap to the subject, and the subject has a hemoglobin (Hgb) level of less than 9.5 g/dL. In yet another embodiment, the non-transfusion dependent or RBC transfusion-independent subject has received no (0) RBC transfusion during a period of time of 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days prior to administration of fedratinib and/or the ActRIIB ligand trap to the subject, and the subject has a hemoglobin (Hgb) level of less than 9.5 g/dL.

In some embodiments, the subject is red blood cell transfusion dependent if the subject has received 4 or more RBC units during a period of time of at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days prior to administration of fedratinib and/or the ActRIIB ligand trap to the subject. In some embodiments, the subject is red blood cell transfusion dependent if the subject has received 4, 5, 6, 7, 8, 9, 10, 11, 12, 13, 14, 15, 16, 17, 18, 19, or 20 RBC units during a period of time of at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days prior to administration of fedratinib and/or the ActRIIB ligand trap to the subject. In some embodiments, the subject is red blood cell transfusion dependent if the subject has received 4 to 16 RBC units during a period of time of at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days prior to administration of fedratinib and/or the ActRIIB ligand trap to the subject. In some embodiments, the subject is red blood cell transfusion dependent if the subject has received 4 to 12 RBC units during a period of at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days prior to administration of fedratinib and/or the ActRIIB ligand trap to the subject. In some embodiments, the subject is red blood cell transfusion dependent if the subject has received 4 to 10 RBC units during a period of at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days prior to administration of fedratinib and/or the ActRIIB ligand trap to the subject. In some embodiments, the subject is red blood cell transfusion dependent if the subject has received 4 to 8 RBC units during a period of at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days prior to administration of fedratinib and/or the ActRIIB ligand trap to the subject. In some embodiments, the subject is red blood cell transfusion dependent if the subject has received 4 to 6 RBC units during a period of at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days prior to administration of fedratinib and/or the ActRIIB ligand trap to the subject.

In some embodiments, the subject is red blood cell transfusion dependent if the subject has received 4 or more RBC units during a period of time of 84 days prior to administration of fedratinib and/or the ActRIIB ligand trap to the subject. In some embodiments, the subject is red blood cell transfusion dependent if the subject has received 4, 5, 6, 7, 8, 9, 10, 11, 12, 13, 14, 15, 16, 17, 18, 19, or 20 RBC units during a period of time of 84 days prior to administration of fedratinib and/or the ActRIIB ligand trap to the subject. In some embodiments, the subject is red blood cell transfusion dependent if the subject has received 4 to 16 RBC units during a period of time of 84 days prior to administration of fedratinib and/or the ActRIIB ligand trap to the subject. In some embodiments, the subject is red blood cell transfusion dependent if the subject has received 4 to 12 RBC units during a period of 84 days prior to administration of fedratinib and/or the ActRIIB ligand trap to the subject. In some embodiments, the subject is red blood cell transfusion dependent if the subject has received 4 to 10 RBC units during a period of 84 days prior to administration of fedratinib and/or the ActRIIB ligand trap to the subject. In some embodiments, the subject is red blood cell transfusion dependent if the subject has received 4 to 8 RBC units during a period of 84 days prior to administration of fedratinib and/or the ActRIIB ligand trap to the subject. In some embodiments, the subject is red blood cell transfusion dependent if the subject has received 4 to 6 RBC units during a period of 84 days prior to administration of fedratinib and/or the ActRIIB ligand trap to the subject. In some embodiments, the subject is red blood cell transfusion dependent if the subject has received 6 to 12, 6 to 10, or 6 to 8 RBC units during a period of 84 days prior to administration of fedratinib and/or the ActRIIB ligand trap to the subject. In some embodiments, the subject is red blood cell transfusion dependent if the subject has received 8 to 12 or 8 to 10 RBC units during a period of 84 days prior to administration of fedratinib and/or the ActRIIB ligand trap to the subject.

In one embodiment, the red blood cell transfusion dependent subject has received 4 or more RBC units during a period of time of at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days prior to administration of fedratinib and/or the ActRIIB ligand trap to the subject, and the subject has a hemoglobin (Hgb) level of less than 11.5 g/dL. In another embodiment, the red blood cell transfusion dependent subject has received 4 to 12 RBC units during a period of at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days prior to administration of fedratinib and/or the ActRIIB ligand trap to the subject, and the subject has a hemoglobin (Hgb) level of less than 11.5 g/dL.

In one embodiments, the RBC transfusion dependent subject has received 4 or more red blood cell units during a period of time of 84 days prior to administration of fedratinib and/or the ActRIIB ligand trap to the subject, and the subject has a hemoglobin (Hgb) level of less than 11.5 g/dL. In another embodiments, the RBC dependent subject has received 4 to 12 red blood cell units during a period of 84 days prior to administration of fedratinib and/or the ActRIIB ligand trap to the subject, and the subject has a hemoglobin (Hgb) level of less than 11.5 g/dL.

In some embodiments, the RBC transfusion dependent subject has no interval of greater than 2, 3, 4, 5, 6, 7, or 8 weeks without RBC transfusion during a period of time of 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days prior to administration of fedratinib and/or the ActRIIB ligand trap to the subject. In some embodiments, the RBC transfusion dependent subject has no interval of greater than 4, 5, 6, or 7 weeks without RBC transfusion during a period of time of 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days prior to administration of fedratinib and/or the ActRIIB ligand trap to the subject. In some embodiments, the RBC transfusion dependent subject has no interval of greater than 6 weeks without RBC transfusion during a period of time of 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days prior to administration of fedratinib and/or the ActRIIB ligand trap to the subject. In some embodiments, the RBC transfusion dependent subject has no interval of greater than 2, 3, 4, 5, 6, 7, or 8 weeks without RBC transfusion during a period of time of 84 days prior to administration of fedratinib and/or the ActRIIB ligand trap to the subject. In some embodiments, the RBC transfusion dependent subject has no interval of greater than 4, 5, 6, or 7 weeks without RBC transfusion during a period of time of 84 days prior to administration of fedratinib and/or the ActRIIB ligand trap to the subject. In some embodiments, the RBC transfusion dependent subject has no interval of greater than 6 weeks without RBC transfusion during a period of time of 84 days prior to administration of fedratinib and/or the ActRIIB ligand trap to the subject.

In one embodiment, the RBC dependent subject has received 4 or more RBC units in RBC transfusion and has no interval of great than 2, 3, 4, 5, 6, 7, or 8 weeks without RBC transfusion during a period of time of 84 days prior to administration of fedratinib and/or the ActRIIB ligand trap to the subject, and the RBC dependent subject has a hemoglobin (Hgb) level of less than 11.5 g/dL. In another embodiment, the RBC dependent subject has received 4 to 12 RBC units in RBC transfusion and has no interval of great than 2, 3, 4, 5, 6, 7, or 8 weeks without RBC transfusion during a period of time of 84 days prior to administration of fedratinib and/or the ActRIIB ligand trap to the subject, and the RBC dependent subject has a hemoglobin (Hgb) level of less than 11.5 g/dL. In yet another embodiment, the RBC dependent subject has received 4 or more RBC units in RBC transfusion and has no interval of great than 6 weeks without RBC transfusion during a period of time of 84 days prior to administration of fedratinib thereof and/or the ActRIIB ligand trap to the subject, and the RBC dependent subject has a hemoglobin (Hgb) level of less than 11.5 g/dL. In yet another embodiment, the RBC dependent subject has received 4 to 12 RBC units in RBC transfusion and has no interval of great than 6 weeks without RBC transfusion during a period of time of 84 days prior to administration of fedratinib and/or the ActRIIB ligand trap to the subject, and the RBC dependent subject has a hemoglobin (Hgb) level of less than 11.5 g/dL.

In certain embodiments, a subject treated in accordance with the methods provided herein has thrombocytopenia. In certain embodiments, a subject treated in accordance with the methods provided herein has less than 1×10¹¹ platelets per liter. In certain embodiments, a subject treated in accordance with the methods provided herein has neutropenia. In certain embodiments, a subject treated in accordance with the methods provided herein has an absolute neutrophil count of less than 1×10⁹ per liter.

In certain embodiments, a subject treated in accordance with the methods provided herein has less than 13,000 white blood cells per μL, less than 12,000 white blood cells per μL, less than 11,000 white blood cells per μL, less than 10,000 white blood cells per μL, less than 7,500 white blood cells per μL, or less than 500 white blood cells per μL.

In certain embodiments, hemoglobin levels in a subject treated in accordance with the methods provide herein are less than 10 g/dL, 9 g/dL, 8 g/dL, or 7 g/dL. In certain embodiments, hemoglobin levels in a subject treated in accordance with the methods provided herein are between 7 g/dL and 7.5 g/dL, between 7.5 g/dL and 8 g/dL, between 8 g/dL and 8.5 g/dL, between 8.5 g/dL and 9.0 g/dL, between 9.0 g/dL and 9.5 g/dL, or between 9.5 g/dL and 10.0/dL.

In certain embodiments, a subject treated in accordance with the methods provided herein has a low transfusion burden. In certain embodiments, the subject with a low transfusion burden treated in accordance with the methods provided herein requires at most 0, 1, 2, or 3 units of red blood cells per 8 weeks. In certain embodiments, a subject treated in accordance with the methods provided herein has a high transfusion burden. In certain embodiments, the subject with a high transfusion burden treated in accordance with the methods provided herein requires at least 4, 5, 6, 7, 8, 9, 10, 11, 12, or 13 units of red blood cells per 8 weeks.

### 5.7 Pharmaceutical Compositions

Also provided herein are “pharmaceutical compositions,” which comprise an ActRIIB ligand trap provided herein or fedratinib or pharmaceutically acceptable salt or hydrate thereof provided herein, and one or more pharmaceutically acceptable carriers. In a particular embodiment, the pharmaceutical composition comprises an ActRIIB ligand trap provided herein. In a particular embodiment, the pharmaceutical composition comprises fedratinib or pharmaceutically acceptable salt or hydrate thereof provided herein, and one or more pharmaceutically acceptable carriers. In a particular embodiment, the ActRIIB ligand trap is present in a therapeutically effective amount. In a particular embodiment, the ActRIIB ligand traps is present in a prophylactically effective amount. The pharmaceutical compositions can be used in accordance with the methods and uses provided herein. Thus, for example, the pharmaceutical compositions can be administered to a subject in order to practice the treatment or prevention methods and uses provided herein. Pharmaceutical compositions provided herein can be formulated to be compatible with the intended method or route of administration; exemplary routes of administration are set forth herein.

Pharmaceutical compositions typically comprise ActRIIB ligand traps provided herein or fedratinib or pharmaceutically acceptable salt or hydrate thereof provided herein, and a pharmaceutically acceptable carrier. Suitable pharmaceutically acceptable carriers include, but are not limited to, antioxidants (e.g., ascorbic acid), preservatives (e.g., benzyl alcohol, methyl parabens, p-hydroxybenzoate), emulsifying agents, suspending agents, dispersing agents, solvents, buffers, lubricants, fillers, and/or diluents. For example, a suitable vehicle may be physiological saline solution. Typical buffers that can be used include, but are not limited to pharmaceutically acceptable weak acids, weak bases, or mixtures thereof. Buffer components can also include water soluble reagents such as phosphoric acid, tartaric acids, succinic acid, citric acid, acetic acid, and salts thereof.

A vehicle may contain other pharmaceutically acceptable excipients for modifying or maintaining the pH, osmolarity, viscosity, or stability of the pharmaceutical composition. In a specific embodiment, the vehicle is an aqueous buffer. In a specific embodiment, a vehicle comprises, for example, sodium chloride.

Pharmaceutical compositions provided herein may contain still other pharmaceutically acceptable formulation agents. Regarding pharmaceutically acceptable formulation agents, see, for example, Remington's Pharmaceutical Sciences, 18th Ed. (1990, Mack Publishing Co., Easton, Pa. 18042) pages 1435-1712, and The Merck Index, 12th Ed. (1996, Merck Publishing Group, Whitehouse, N.J.).

In certain embodiments the ActRIIB ligand traps and fedratinib are formulated suitable for combination treatment described herein.

In certain embodiments, the ActRIIB ligand traps are formulated with a pharmaceutically acceptable carrier for use with the methods described herein. For example, an ActRIIB ligand trap can be administered alone or as a component of a pharmaceutical formulation (therapeutic composition). The ActRIIB ligand traps may be formulated for administration in any convenient way for use in human or veterinary medicine.

In a preferred embodiment, the ActRIIB ligand trap is formulated for subcutaneous administration.

In another preferred embodiment, the ActRIIB ligand trap is packaged in a container as a sterile, preservative-free lyophilized powder or cake. In certain embodiments, the container comprises 25 mg of the ActRIIB ligand trap. In certain embodiments, the container comprising 25 mg of the ActRIIB ligand trap comprises a total of 37.5 mg of protein. In certain embodiments, ActRIIB ligand trap in the container comprising 25 mg of the ActRIIB ligand trap is reconstituted with 0.68 mL of water for injection. In certain embodiments, the container comprises 75 mg of the ActRIIB ligand trap. In certain embodiments, the container comprising 75 mg of the ActRIIB ligand trap comprises a total of 87.5 mg of protein. In certain embodiments, ActRIIB ligand trap in the container comprising 75 mg of the ActRIIB ligand trap is reconstituted with 1.6 mL of water for injection. In certain embodiments, the ActRIIB ligand trap in the container is reconstituted with a volume of water for injection, such that the final concentration of the reconstituted ActRIIB ligand trap in the water for injection is 50 mg/mL with a pH of approximately 6.5. In certain embodiments, the ActRIIB ligand trap is administered to a subject within 10 hours of reconstitution. In certain embodiments, the container comprises the ActRIIB ligand trap at a concentration of 50 mg/mL in a 10 mM citrate buffer-based solution, wherein the 10 mM citrate-buffer based solution comprises 10 mM citrate, pH 6.5, 9% sucrose, and 0.02% polysorbate 80. In certain embodiments, the container is stored at between 2° C. and 8° C. In certain embodiments, the container is stored at between 2° C. and 8° C. for 18 months. In certain embodiments, the container is a 3 mL glass vial with a gray butyl coated stopper. In certain embodiments, the container is a 3 mL glass vial with a gray rubber stopper. In certain embodiments, the rubber stopper is secured in place by a crimped aluminum flip cap with a colored plastic button. In certain embodiments, the 3 mL glass vial comprises 25 mg of the ActRIIB ligand trap and the colored plastic button is red. In certain embodiments, 3 mL glass vial comprises 75 mg of the ActRIIB ligand trap and the colored plastic button is white. In specific embodiments, the ActRIIB ligand trap is a polypeptide comprising the amino acid sequence of SEQ ID NO:11. In specific embodiments, the ActRIIB ligand trap is a polypeptide comprising an amino acid sequence consisting of the amino acid sequence of SEQ ID NO:11. In specific embodiments, the ActRIIB ligand trap is a polypeptide consisting of the amino acid sequence set forth in SEQ ID NO:11.

In a specific embodiment, the ActRIIB ligand trap is packaged in a container as a sterile, preservative-free lyophilized powder or cake. In a specific embodiment, the container comprises 50 mg/mL of ActRIIB ligand trap in 10 mM citrate buffer pH 6.5. In a specific embodiment, the container comprises 56 mg of ActRIIB ligand trap, 0.19 mg of citric acid monohydrate, 3.03 mg of tri-sodium citrate dehydrate, 0.24 mg of polysorbate 80, and 100.80 mg of sucrose. In specific embodiments, the ActRIIB ligand trap is a polypeptide comprising the amino acid sequence of SEQ ID NO:11. In specific embodiments, the ActRIIB ligand trap is a polypeptide comprising an amino acid sequence consisting of the amino acid sequence of SEQ ID NO:11. In specific embodiments, the ActRIIB ligand trap is a polypeptide consisting of the amino acid sequence set forth in SEQ ID NO:11.

In certain embodiments, the therapeutic methods provided herein include administering the composition (comprising an ActRIIB ligand trap) systemically, or locally as an implant or device. When administered, the therapeutic composition for uses provided herein is in a pyrogen-free, physiologically acceptable form. Therapeutically useful agents other than the ActRIIB ligand traps which may also optionally be included in the composition as described above, may be administered simultaneously or sequentially with the subject compounds (e.g., ActRIIB ligand traps described in Section 5.4).

Typically, ActRIIB ligand traps will be administered parenterally. In a preferred embodiment, the ActRIIB ligand trap will be administered subcutaneously. Pharmaceutical compositions suitable for parenteral administration may comprise one or more ActRIIB polypeptides in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions for use in the methods described herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

The compositions described herein may also contain adjuvants, such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

In certain embodiments, the ActRIIB ligand trap is substantially pure in a pharmaceutical composition. Specifically, at most 20%, 10%, 5%, 2.5%, 1%, 0.1%, or at most 0.05% of the compounds in the pharmaceutical composition are compounds other than the ActRIIB ligand trap and the pharmaceutical acceptable carrier.

In certain embodiments, the ActRIIB ligand trap is administered at room temperature to a patient according to a method provided herein.

In certain embodiments, fedratinib is formulated with a pharmaceutically acceptable carrier for use with the methods described herein.

In certain embodiments, fedratinib can be administered to a subject orally, topically or parenterally in the conventional form of preparations, such as capsules, microcapsules, tablets, granules, powder, troches, pills, suppositories, injections, suspensions, syrups, patches, creams, lotions, ointments, gels, sprays, solutions and emulsions. Suitable formulations can be prepared by methods commonly employed using conventional, organic or inorganic additives, such as a diluent (e.g., sucrose, starch, mannitol, sorbitol, lactose, glucose, cellulose, talc, calcium phosphate or calcium carbonate), a binder (e.g., cellulose, methylcellulose, hydroxymethylcellulose, polypropylpyrrolidone, polyvinylpyrrolidone, gelatin, gum arabic, polyethyleneglycol, sucrose or starch), a disintegrant (e.g., starch, carboxymethylcellulose, hydroxypropylstarch, low substituted hydroxypropylcellulose, sodium bicarbonate, calcium phosphate or calcium citrate), a lubricant (e.g., magnesium stearate, light anhydrous silicic acid, talc or sodium lauryl sulfate), a flavoring agent (e.g., citric acid, menthol, glycine or orange powder), a preservative (e.g., sodium benzoate, sodium bisulfite, methylparaben or propylparaben), a stabilizer (e.g., citric acid, sodium citrate or acetic acid), a suspending agent (e.g., methylcellulose, polyvinyl pyrroliclone or aluminum stearate), a dispersing agent (e.g., hydroxypropylmethylcellulose), water, and base wax (e.g., cocoa butter, white petrolatum or polyethylene glycol). The effective amount of the compounds in the pharmaceutical composition may be at a level that will exercise the desired effect for both oral and parenteral administration.

In a preferred embodiment, fedratinib is formulated for oral administration.

In certain embodiments, fedratinib is formulated for parental administration. In one embodiment, fedratinib is packaged in a container as a sterile, preservative-free lyophilized powder or cake.

In certain embodiments, fedratinib is formulated in the form of a capsule or tablet. In certain embodiments, fedratinib is formulated in the form of microtablets or micropellets, and wherein the microtablets or micropellets are enterically coated. In certain embodiments, the microtablets or micropellets are contained in a capsule. In a preferred embodiment, fedratinib is formulated in the form of a capsule.

### 5.8 Doses and Regimen

### 5.8.1 Doses of ActRIIB Ligand Trap

In certain embodiments, the ActRIIB ligand trap administered to a subject according to the methods provided herein (see Sections 5.5 and 5.9) is a product resulting from expression from an opening reading frame with the nucleotide sequence of SEQ ID NO:34 or a degenerate version of SEQ ID NO:34 that encodes SEQ ID NO:11. In certain embodiments, the dose of the ActRIIB ligand trap (e.g. a product resulting from expression from an opening reading frame with the nucleotide sequence of SEQ ID NO:34 or a degenerate version of SEQ ID NO:34 that encodes SEQ ID NO:11) is about 0.3 mg/kg to 2.0 mg/kg. In certain embodiments, the dose of the ActRIIB ligand trap (e.g. a product resulting from expression from an opening reading frame with the nucleotide sequence of SEQ ID NO:34 or a degenerate version of SEQ ID NO:34 that encodes SEQ ID NO:11) is about 0.30 mg/kg, 0.35 mg/kg, is about 0.4 mg/kg, about 0.45 mg/kg, about 0.5 mg/kg, about 0.6 mg/kg, about 0.7 mg/kg, about 0.8 mg/kg, about 0.9 mg/kg, about 1.0 mg/kg, or about 1.25 mg/kg. In certain embodiments, the dose of the ActRIIB ligand trap is about 0.3 mg/kg, 0.33 mg/kg, 0.45 mg/kg, 0.6 mg/kg, 0.8 mg/kg, 1.0 mg/kg, 1.33 mg/kg, 1.75 mg/kg, or 2.0 mg/kg. In certain embodiments, the dose of ActRIIB ligand trap is about 0.3 mg/kg. In certain embodiments, the dose of ActRIIB ligand trap is about 0.33 mg/kg. In certain embodiments, the dose of ActRIIB ligand trap is about 0.45 mg/kg. In certain embodiments, the dose of ActRIIB ligand trap is about 0.6 mg/kg. In certain embodiments, the dose of ActRIIB ligand trap is about 0.8 mg/kg. In certain embodiments, the dose of ActRIIB ligand trap is about 1.0 mg/kg. In certain embodiments, the dose of ActRIIB ligand trap is about 1.33 mg/kg. In certain embodiments, the dose of ActRIIB ligand trap is about 1.75 mg/kg. In certain embodiments, the dose of ActRIIB ligand trap is about 2.0 mg/kg. In some embodiments, the ActRIIB ligand trap is administered at a pharmaceutically effective amount. In one embodiment, the pharmaceutically effective amount of the ActRIIB ligand trap administered is 1.33 mg/kg.

In certain embodiments, the dose of ActRIIB ligand trap is an initial dose. In certain embodiments, the initial dose of ActRIIB ligand trap is about 0.3 mg/kg. In certain embodiments, the initial dose of ActRIIB ligand trap is about 0.33 mg/kg. In certain embodiments, the initial dose of ActRIIB ligand trap is about 0.45 mg/kg. In certain embodiments, the initial dose of ActRIIB ligand trap is about 0.6 mg/kg. In certain embodiments, the initial dose of ActRIIB ligand trap is about 0.8 mg/kg. In certain embodiments, the dose of ActRIIB ligand trap is about 1.0 mg/kg. In preferred embodiments, the initial dose of ActRIIB ligand trap is about 1.33 mg/kg. In certain embodiments, the initial dose of ActRIIB ligand trap is about 1.75 mg/kg. In certain embodiments, the initial dose of ActRIIB ligand trap is about 2.0 mg/kg.

In certain embodiments, the dose of the ActRIIB ligand trap is a subsequent dose. In some embodiments, the subsequent dose of the ActRIIB ligand trap is 0.30 mg/kg, 0.33 mg/kg, 0.45 mg/kg, 0.6 mg/kg, 0.8 mg/kg, 1.0 mg/kg, 1.33 mg/kg, 1.75 mg/kg, or 2.0 mg/kg. In some embodiments, the subsequent dose of the ActRIIB ligand trap is 0.6 mg/kg, 0.8 mg/kg, 1.0 mg/kg, 1.33 mg/kg, or 1.75 mg/kg. In some embodiments, the subsequent dose of the ActRIIB ligand trap is 0.30 mg/kg. In some embodiments, the subsequent dose of the ActRIIB ligand trap is 0.33 mg/kg. In some embodiments, the subsequent dose of the ActRIIB ligand trap is 0.45 mg/kg. In some embodiments, the subsequent dose of the ActRIIB ligand trap is 0.6 mg/kg. In some embodiments, the subsequent dose of the ActRIIB ligand trap is 0.8 mg/kg. In some embodiments, the subsequent dose of the ActRIIB ligand trap is 1.0 mg/kg. In some embodiments, the subsequent dose of the ActRIIB ligand trap is 1.33 mg/kg. In some embodiments, the subsequent dose of the ActRIIB ligand trap is 1.75 mg/kg. In some embodiments, the subsequent dose of the ActRIIB ligand trap is 2.0 mg/kg.

In certain embodiments, the subsequent dose of the ActRIIB ligand trap is about 0.5 mg, about 0.5 mg, about 0.6 mg, about 0.7 mg, about 0.8 mg, about 0.9 mg, about 1 mg, about 1.1 mg, about 1.2 mg, about 1.3 mg, about 1.4 mg, about 1.5 mg, about 1.6 mg, about 1.7 mg, about 1.8 mg, about 1.9 mg, about 2.0 mg, about 2.1 mg, about 2.2 mg, about 2.3 mg, about 2.4 mg, about 2.5 mg, about 2.5 mg, about 2.6 mg, about 2.7 mg, about 2.8 mg, about 2.9 mg, about 3.0 mg, about 3.5 mg, about 4.0 mg, about 4.5 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, or about 35 mg greater than the initial dose of the ActRIIB ligand trap. In certain embodiments, the subsequent dose of the ActRIIB ligand trap is about 0.05 mg/kg, about 0.06 mg/kg, about 0.07 mg/kg, about 0.08 mg/kg, about 0.09 mg/kg, about 0.1 mg/kg, about 0.11 mg/kg, about 0.12 mg/kg, about 0.13 mg/kg, about 0.14 mg/kg, about 0.15 mg/kg, about 0.16 mg/kg, about 0.17 mg/kg, about 0.18 mg/kg, about 0.19 mg/kg, about 0.20 mg/kg, about 0.21 mg/kg, about 0.22 mg/kg, about 0.23 mg/kg, about 0.24 mg/kg, about 0.25 mg/kg, about 0.26 mg/kg, about 0.27 mg/kg, about 0.28 mg/kg, about 0.29 mg/kg, about 0.3 mg/kg, about 0.35 mg/kg, about 0.4 mg/kg, about 0.45 mg/kg, or about 0.5 mg/kg greater than the initial dose of the ActRIIB ligand trap.

In certain embodiments, the subsequent dose is about 0.5 mg, about 0.5 mg, about 0.6 mg, about 0.7 mg, about 0.8 mg, about 0.9 mg, about 1 mg, about 1.1 mg, about 1.2 mg, about 1.3 mg, about 1.4 mg, about 1.5 mg, about 1.6 mg, about 1.7 mg, about 1.8 mg, about 1.9 mg, about 2.0 mg, about 2.1 mg, about 2.2 mg, about 2.3 mg, about 2.4 mg, about 2.5 mg, about 2.5 mg, about 2.6 mg, about 2.7 mg, about 2.8 mg, about 2.9 mg, about 3.0 mg, about 3.5 mg, about 4.0 mg, about 4.5 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, or about 35 mg greater than the initial dose, or about 0.05 mg/kg, about 0.06 mg/kg, about 0.07 mg/kg, about 0.08 mg/kg, about 0.09 mg/kg, about 0.1 mg/kg, about 0.11 mg/kg, about 0.12 mg/kg, about 0.13 mg/kg, about 0.14 mg/kg, about 0.15 mg/kg, about 0.16 mg/kg, about 0.17 mg/kg, about 0.18 mg/kg, about 0.19 mg/kg, about 0.20 mg/kg, about 0.21 mg/kg, about 0.22 mg/kg, about 0.23 mg/kg, about 0.24 mg/kg, about 0.25 mg/kg, about 0.26 mg/kg, about 0.27 mg/kg, about 0.28 mg/kg, about 0.29 mg/kg, about 0.3 mg/kg, about 0.35 mg/kg, about 0.4 mg/kg, about 0.45 mg/kg, or about 0.5 mg/kg less than the initial dose.

In certain embodiments, the subsequent dose is administered more frequently than the initial dose. In certain embodiments, the subsequent dose is administered less frequently than the initial dose. In certain embodiments, the subsequent dose is administered at the same frequency as the initial dose. In certain embodiments, the subsequent dose is administered every 14, 15, 16, 17, 18, 19, 20, 21, 22, 23, 24, 25, 26, 27, or 28 days. In certain embodiments, the subsequent dose is administered every 21 days. In certain embodiments, the subsequent dose is administered continuously and/or indefinitely.

In some embodiments, the ActRIIB ligand trap is administered to the subject once every 14 days, 21 days, once every 28 days, every 35 days, or once every 42 days. In preferred embodiments, the ActRIIB ligand trap is administered to the subject once every 21 days. In some embodiments, the ActRIIB ligand trap is administered to the subject 1, 2, or 3 times every treatment cycle. In preferred embodiments, the ActRIIB ligand trap is administered to the subject once every treatment cycle. In some embodiments, the ActRIIB ligand trap is administered to the subject for at least 1, 2, 3, 4, 5, 6, 7, 8, 9, or 10 cycles. In one embodiment, the ActRIIB ligand trap is administered to the subject for at least 6, 7, 8, 9, or 10 cycles. In another embodiment, the ActRIIB ligand trap is administered to the subject for 6 cycles. In yet another embodiment, the ActRIIB ligand trap is administered to the subject for 7 cycles. In yet another embodiment, the ActRIIB ligand trap is administered to the subject for 8 cycles. In yet another embodiment, the ActRIIB ligand trap is administered to the subject for 9 cycles. In yet another embodiment, the ActRIIB ligand trap is administered to the subject for 10 cycles. In one embodiment, the ActRIIB ligand trap is administered once at the beginning of every treatment cycle.

In certain embodiments, the subsequent dose is administered more frequently than the initial dose. In certain embodiments, the subsequent dose is administered less frequently than the initial dose. In certain embodiments, the subsequent dose is administered at the same frequency as the initial dose. In certain embodiments, the subsequent dose is administered every 14, 15, 16, 17, 18, 19, 20, 21, 22, 23, 24, 25, 26, 27, or 28 days. In certain embodiments, the subsequent dose is administered every 21 days. In certain embodiments, the subsequent dose is administered continuously and/or indefinitely.

In certain embodiments, the ActRIIB ligand trap is administered to the subject subcutaneously. In certain embodiments, the ActRIIB ligand trap is administered to the subject subcutaneously in the upper arm, abdomen, or thigh of the subject. In certain embodiments, the ActRIIB ligand trap is administered to the subject every 21 days. In certain embodiments, the ActRIIB ligand trap is administered to the subject every 14, 15, 16, 17, 18, 19, 20, 21, 22, 23, 24, 25, 26, 27, or 28 days. In certain embodiments, the ActRIIB ligand trap is administered to the subject every 21 days, subcutaneously in the upper arm, abdomen, or thigh of the subject.

In certain embodiments, the ActRIIB ligand trap is part of a composition as described in Section 5.7. In certain embodiments, the ActRIIB ligand trap is a sterile, preservative-free, lyophilized powder reconstituted in water for injection. In certain embodiments, a single dose of the ActRIIB ligand trap is reconstituted in a volume of water for injection of greater than 1 mL. In such embodiments, the single dose of the ActRIIB ligand trap is administered to the subject via two injections of equal volume of reconstituted ActRIIB ligand inhibitor. In certain embodiments, the two injections are administered to the subject at separate sites, e.g., one injection in the right thigh and one injection in the left thigh.

### 5.8.2 Doses of Fedratinib

In certain embodiments, fedratinib or pharmaceutically acceptable salt or hydrate thereof is administered to the subject orally. In certain embodiments, fedratinib or pharmaceutically acceptable salt or hydrate thereof is administered to the subject daily. In certain embodiments, fedratinib or pharmaceutically acceptable salt or hydrate thereof is administered to the subject every 1, 2, 3, 4, 5, 6, 7, 8, 9, 10, 11, 12, 13, 14, 15, 16, 17, 18, 19, 20, 21, 22, 23, 24, 25, 26, 27, or 28 days.

In some embodiments, fedratinib or pharmaceutically acceptable salt or hydrate thereof is administered to the subject at a dose of 600 mg/day, 500 mg/day, 400 mg/day, 300 mg/day, 200 mg/day, or 100 mg/day.

In some embodiment, fedratinib or pharmaceutically acceptable salt or hydrate thereof is administered to the subject at an initial dose of 400 mg/day, 300 mg/day, 200 mg/day, or 100 mg/day. In one preferred embodiment, the initial dose is 400 mg/day. In another embodiment, the initial dose is 300 mg/day. In yet another embodiment, the initial dose is 200 mg/day. In yet another embodiment, the initial dose is 100 mg/day.

In some embodiment, fedratinib or pharmaceutically acceptable salt or hydrate thereof is administered to the subject at a subsequent dose of 400 mg/day, 300 mg/day, 200 mg/day, or 100 mg/day. In certain embodiments, the subsequent dose of fedratinib is determined according to the methods provided in Section 5.5. In one preferred embodiment, the subsequent dose is 400 mg/day. In another embodiment, the subsequent dose is 300 mg/day. In yet another embodiment, the subsequent dose is 200 mg/day. In yet another embodiment, the subsequent dose is 100 mg/day.

### 5.9 Combination Therapy

In certain embodiments, fedratinib or pharmaceutically acceptable salt and/or hydrate thereof is administered before or concurrently with the administration of the ActRIIB ligand trap.

In certain embodiments, fedratinib or pharmaceutically acceptable salt or hydrate thereof is administered after the administration of the ActRIIB ligand trap.

In certain embodiments, the subject has been previously treated with fedratinib or pharmaceutically acceptable salt or hydrate thereof prior to the administration of the ActRIIB ligand trap.

In certain embodiments, the subject has been previously treated with the ActRIIB ligand trap prior to the administration of fedratinib or pharmaceutically acceptable salt or hydrate thereof.

In certain embodiments, the subject has been previously treated with fedratinib or pharmaceutically acceptable salt or hydrate thereof prior to the co-administration of the ActRIIB ligand trap and fedratinib or pharmaceutically acceptable salt or hydrate thereof.

In certain embodiments, the co-administration of the ActRIIB ligand trap and fedratinib or pharmaceutically acceptable salt or hydrate thereof is concomitant administration.

In certain embodiments, the ActRIIB ligand trap and fedratinib or pharmaceutically acceptable salt or hydrate thereof are concomitantly administered.

In certain embodiments, the co-administration of the ActRIIB ligand trap and fedratinib or pharmaceutically acceptable salt or hydrate thereof is pharmaceutically effective to treat anemia.

In certain embodiments, the co-administration of the ActRIIB ligand trap and fedratinib or pharmaceutically acceptable salt or hydrate thereof is pharmaceutically effective to treat anemia, wherein the respective doses of the ActRIIB ligand trap and fedratinib are doses sufficient to reduce or palliate one or more symptoms of anemia. In certain embodiments, the co-administration of the ActRIIB ligand trap and fedratinib or pharmaceutically acceptable salt or hydrate thereof is pharmaceutically effective to treat anemia, wherein the respective doses of the ActRIIB ligand trap and fedratinib are doses sufficient to reduce or palliate one or more symptoms of anemia caused by or associated with myeloproliferative neoplasm-associated myelofibrosis. In certain embodiments, the co-administration of the ActRIIB ligand trap and fedratinib is pharmaceutically effective to prevent at least one symptom of anemia from worsening.

In certain embodiments, combination treatment of the ActRIIB ligand trap and fedratinib show synergistic effects in treating anemia. In certain embodiments, the dose of the ActRIIB ligand trap in combination treatment with fedratinib is lower than the pharmaceutically effective dose of the ActRIIB ligand trap in a monotherapy. In certain embodiments, the dose of fedratinib in combination treatment is lower than the pharmaceutically effective dose of fedratinib in a monotherapy.

In certain embodiments, the co-administration of the ActRIIB ligand trap and fedratinib or pharmaceutically acceptable salt or hydrate thereof is pharmaceutically effective to increase the level of hemoglobin in the subject as compared to the level of hemoglobin in the subject at baseline. In the context of evaluating the increase in the level of hemoglobin in the subject, “baseline” refers to the time immediately prior to the first administration of fedratinib and/or the ActRIIB ligand trap to the subject. In certain embodiments, the co-administration of the ActRIIB ligand trap and fedratinib or pharmaceutically acceptable salt or hydrate thereof is pharmaceutically effective to increase the level of hemoglobin (Hgb) in the subject by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% over any consecutive 84-day period after the subject has been administered an initial dose of the ActRIIB ligand trap. In certain embodiments, the co-administration of the ActRIIB ligand trap and fedratinib or pharmaceutically acceptable salt or hydrate thereof is pharmaceutically effective, wherein the subject has been transfused less than 4 units of red blood cell units during the consecutive 84-day period. In certain embodiments, the co-administration of the ActRIIB ligand trap and fedratinib or pharmaceutically acceptable salt or hydrate thereof is pharmaceutically effective, wherein the subject has been transfused 0 units of red blood cell units during the consecutive 84-day period. In certain embodiments, the co-administration of the ActRIIB ligand trap and fedratinib or pharmaceutically acceptable salt or hydrate thereof is pharmaceutically effective to increase the level of hemoglobin by at least 0.5 g/dL, at least 0.8 g/dL, at least 1.0 g/dL, at least 1.2 g/dL, at least 1.5 g/dL, at least 1.8 g g/dL, at least 2.0 g/dL, at least 2.2 g/dL, at least 2.4 g/dL, at least 2.6 g/dL, at least 2.8 g/dL, at least 3.0 g/dL, at least 3.2 g/dL, at least 3.4 g/dL, at least 3.6 g/dL, at least 3.8 g/dL, at least 4.0 g/dL, at least 5.0 g/dL, or at least 6.0 g/dL over any consecutive 84-day period after the subject has been administered an initial dose of the ActRIIB ligand trap. In certain embodiments, the co-administration of the ActRIIB ligand trap and fedratinib or pharmaceutically acceptable salt or hydrate thereof is pharmaceutically effective to increase hemoglobin level in the subject for at least 3, at least 4, at least 5, at least 6, at least 12, at least 18, at least 24 or at least 48 months after administration of the ActRIIB ligand trap. In certain embodiments, the co-administration of the ActRIIB ligand trap and fedratinib or pharmaceutically acceptable salt or hydrate thereof is pharmaceutically effective to decrease the frequency of red blood cell transfusion in the subject as compared to the frequency of red blood cell transfusion in the subject at baseline.

In some embodiments, the co-administration of the ActRIIB ligand trap and fedratinib is pharmaceutically effective to reduce RBC transfusion burden of the subject by at least 10%, at least 20%, at least 30%, at least 40%, at least 50%, at least 60%, at least 70%, at least 80%, at least 90%, or at least 100%. In some embodiments, the co-administration of the ActRIIB ligand trap and fedratinib is pharmaceutically effective to reduce RBC transfusion burden of the subject by at least 40%. In some embodiments, the co-administration of the ActRIIB ligand trap and fedratinib is pharmaceutically effective to reduce RBC transfusion burden of the subject by at least 50%. In some embodiments, the co-administration of the ActRIIB ligand trap and fedratinib is pharmaceutically effective to reduce RBC transfusion burden of the subject by at least 60%. In some embodiments, the co-administration of the ActRIIB ligand trap and fedratinib is pharmaceutically effective to reduce RBC transfusion burden of the subject by at least 70%.

In some embodiments, the co-administration of the ActRIIB ligand trap and fedratinib is pharmaceutically effective to reduce at least 2, at least 3, at least 4, at least 5, at least 6, at least 7, at least 8, at least 9, or at least 10 units of red blood cell (RBC) transfusions received by the subject within a period of 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days. In some embodiments, the co-administration of the ActRIIB ligand trap and fedratinib is pharmaceutically effective reduce at least 4 units of red blood cell (RBC) transfusions received by the subject within a period of 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days. In some embodiments, the co-administration of the ActRIIB ligand trap and fedratinib is pharmaceutically effective to reduce at least 8 units of red blood cell (RBC) transfusions received by the subject within a period of 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days. In some embodiments, the co-administration of the ActRIIB ligand trap and fedratinib is pharmaceutically effective to reduce at least 4 units of red blood cell (RBC) transfusions received by the subject within a period of 56 days. In some embodiments, the co-administration of the ActRIIB ligand trap and fedratinib is pharmaceutically effective to reduce at least 4 units of red blood cell (RBC) transfusions received by the subject within a period of 63 days. In some embodiments, the co-administration of the ActRIIB ligand trap and fedratinib is pharmaceutically effective to reduce at least 4 units of red blood cell (RBC) transfusions received by the subject within a period of 70 days. In some embodiments, the co-administration of the ActRIIB ligand trap and fedratinib is pharmaceutically effective to reduce at least 4 units of red blood cell (RBC) transfusions received by the subject within a period of 77 days. In some embodiments, the co-administration of the ActRIIB ligand trap and fedratinib is pharmaceutically effective to reduce at least 4 units of red blood cell (RBC) transfusions received by the subject within a period of 84 days.

In some embodiments, the co-administration of the ActRIIB ligand trap and fedratinib is pharmaceutically effective, wherein the subject becomes RBC transfusion free or non-transfusion dependent over a consecutive period of 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 98, or 105 days. In some embodiments, the co-administration of the ActRIIB ligand trap and fedratinib is pharmaceutically effective, wherein the subject becomes RBC transfusion free over a consecutive period of 84 days. In some embodiments, the co-administration of the ActRIIB ligand trap and fedratinib is pharmaceutically effective, wherein the subject becomes RBC transfusion free over a consecutive period of 91 days. In some embodiments, the co-administration of the ActRIIB ligand trap and fedratinib is pharmaceutically effective, wherein the subject becomes RBC transfusion free over a consecutive period of 105 days. In some embodiments, the co-administration of the ActRIIB ligand trap and fedratinib is pharmaceutically effective, wherein the subject becomes non-transfusion dependent over a consecutive period of 84 days. In some embodiments, the co-administration of the ActRIIB ligand trap and fedratinib is pharmaceutically effective, wherein the subject becomes non-transfusion dependent over a consecutive period of 91 days. In some embodiments, the co-administration of the ActRIIB ligand trap and fedratinib is pharmaceutically effective, wherein the subject becomes non-transfusion dependent over a consecutive period of 105 days.

In some embodiments, the co-administration of the ActRIIB ligand trap and fedratinib is pharmaceutically effective to increase the hemoglobin (Hgb) level in the subject by at least 1.5 g/dL or reduce at least 4 units of red blood cell (RBC) transfusions received by the subject within a period of 56 days compared with baseline, wherein the subject is red blood cell transfusion dependent or non-transfusion dependent.

In some embodiments, the co-administration of the ActRIIB ligand trap and fedratinib is pharmaceutically effective to reduce RBC transfusion burden of the subject by at least 50% and reduce at least 4 units of red blood cell (RBC) transfusions received by the subject within a period of 84 days from baseline, wherein the subject is red blood cell transfusion dependent.

In some embodiments, the co-administration of the ActRIIB ligand trap and fedratinib is pharmaceutically effective to increase the hemoglobin (Hgb) level in the subject by at least 1.5 g/dL compared with baseline and the subject becomes RBC transfusion free over a consecutive period of 84 days, wherein the subject is non-transfusion dependent. In specific embodiments, the co-administration of the ActRIIB ligand trap and fedratinib is pharmaceutically effective to increase the hemoglobin (Hgb) level in the subject by at least 1.5 g/dL compared with baseline over a consecutive period of 84 days without an RBC transfusion (transfusion free), wherein the subject is non-transfusion dependent.

In some embodiments, the co-administration of the ActRIIB ligand trap and fedratinib is pharmaceutically effective, wherein the subject becomes RBC transfusion free over a consecutive period of 84 days, wherein the subject is red blood cell transfusion dependent or non-transfusion dependent.

In certain embodiments, one RBC unit refers to about 150 mL, 200 mL, 250 mL, 300 mL, 350 mL, 100-200 mL, 150-250 mL, 200-300 mL, 250-300 mL, or 250-350 mL of RBCs.

### 5.9.1 Clinical Benefit of Combination

Therapy with JAK2 inhibitors (e.g., ruxolitinib or fedratinib) can be associated with moderate to severe thrombocytopenia and anemia. Dose reduction and treatment discontinuation are used to address these side effects (Verstovsek, 2017, J. Hematol. Oncol. 10:55).

Also provided herein are methods of safely administering a JAK2 inhibitor for treatment of myelofibrosis in a subject without dose reduction, said method comprising: administering an activin receptor type IIB (ActRIIB) ligand trap to the subject (e.g., luspatercept) and administering a JAK2 inhibitor to the subject. Without being bound by theory, the ActRIIB ligand trap eliminates or reduces one or more of the side effects associated with JAK2 therapy (such as thrombocytopenia or anemia).

In certain embodiments, the JAK2 inhibitor is ruxolitinib. In certain embodiments, the JAK2 inhibitor is fedratinib. In some embodiments, the JAK2 inhibitor is administered at a normal dose. In some embodiments, the normal dose of the JAK2 inhibitor is the recommended starting dose. In one embodiment, when the JAK2 inhibitor is ruxolitinib, the normal dose is 20 mg twice daily. In another embodiment, when the JAK2 inhibitor is ruxolitinib, the normal dose is 15 mg twice daily. In one embodiment, when the JAK2 inhibitor is fedratinib, the normal dose is 400 mg once daily. In another embodiment, when the JAK2 inhibitor is fedratinib, the normal dose is 300 mg once daily. In yet another embodiment, when the JAK2 inhibitor is fedratinib, the normal dose is 200 mg once daily. In certain embodiments, the JAK2 inhibitor is administered at least 75%, 80%, 85%, 90%, or 95% of the recommended starting dose. In certain embodiments, the normal dose is an adjusted normal dose when concomitant strong CYP3A4 inhibitor is administered. In certain embodiments, the recommended starting dose is an adjusted recommended starting dose when concomitant strong CYP3A4 inhibitor is administered.

In certain embodiments, the JAK2 inhibitor is administered at the normal dose for an extended period of time. In certain embodiments, the JAK2 inhibitor is administered at the normal dose for at least 1, 2, 3, 4, 5, 6, 7, 8, 9, 10, 11, or 12 months. In certain embodiments, the JAK2 inhibitor is administered at the normal dose for at least 1, 2, 3, 4, 5, 6, 7, 8, 9, or 10 years.

In certain embodiments, the dose of the JAK2 inhibitor is not reduced due to adverse effects when the activin receptor type IIB (ActRIIB) ligand trap is concomitantly administered. In certain embodiments, the adverse effect is anemia. In certain embodiments, the adverse effect is thrombocytopenia. In certain embodiments, the adverse effect is neutropenia.

In certain embodiments, the method further comprises monitoring any adverse effects caused by JAK2 inhibitor. In certain embodiments, the adverse effect is anemia. In certain embodiments, the adverse effect is thrombocytopenia. In certain embodiments, the adverse effect is neutropenia. In certain embodiments, the complete blood count (CBC), red blood cell count, or platelet count of the subject is monitored. In one embodiment, the complete blood count (CBC), red blood cell count, or platelet count of the subject is measured every month. In another embodiment, the complete blood count (CBC), red blood cell count, or platelet count of the subject is measured every 1, 2, 3, 4, 5, 6, 7, 8, 9 or 10 weeks. In another embodiment, the complete blood count (CBC), red blood cell count, or platelet count of the subject is measured 1, 2, 3, or 4 times per week.

In certain embodiments, the myelofibrosis is a myeloproliferative neoplasms (MPN)-associated myelofibrosis. In certain embodiments, the myelofibrosis is intermediate or High-Risk Primary Myelofibrosis (PMF), Post-Polycythemia Vera Myelofibrosis (post-PV MF), or Post-Essential Thrombocythemia Myelofibrosis (post-ET MF).

Also provided herein are methods for reducing adverse effects caused by administration of a JAK2 inhibitor for treatment of myelofibrosis in a subject, said method comprising administering an activin receptor type IIB (ActRIIB) ligand trap to the subject. In certain embodiments, the adverse effect is anemia. In certain embodiments, the adverse effect is thrombocytopenia. In certain embodiments, the adverse effect is neutropenia. In certain embodiments, the dose of the JAK2 inhibitor is not reduced due to adverse effects when the activin receptor type IIB (ActRIIB) ligand trap is concomitantly administered. In certain embodiments, the adverse effect is monitored by measuring the complete blood count (CBC), red blood cell count, or platelet count of the subject. In one embodiment, the complete blood count (CBC), red blood cell count, or platelet count of the subject is measured every month. In another embodiment, the complete blood count (CBC), red blood cell count, or platelet count of the subject is measured every 1, 2, 3, 4, 5, 6, 7, 8, 9 or 10 weeks. In another embodiment, the complete blood count (CBC), red blood cell count, or platelet count of the subject is measured 1, 2, 3, or 4 times per week.

In some embodiments, the ActRIIB ligand trap (e.g. luspatercept) is administered according to the dose and regimen of the administration as described in Sections 5.5 and 5.8. In some embodiments, the pharmaceutically effective amount of the ActRIIB ligand trap administered is 0.6 mg/kg, 0.8 mg/kg, 1 mg/kg, 1.33 mg/kg, or 1.75 mg/kg. In some embodiments, the pharmaceutically effective amount of the ActRIIB ligand trap administered is 1.33 mg/kg. In some embodiments, the initial dose of the ActRIIB ligand trap is 1.33 mg/kg. In some embodiments, the subsequent dose of the ActRIIB ligand trap is 0.6 mg/kg, 0.8 mg/kg, 1.0 mg/kg, 1.33 mg/kg, or 1.75 mg/kg. In some embodiments, the subsequent dose of the ActRIIB ligand trap is 1.33 mg/kg. In some embodiments, the ActRIIB ligand trap is administered once at the beginning of every treatment cycle, wherein each cycle is 21 days. In some embodiments, the ActRIIB ligand trap is administered to the subject for at least 1, 2, 3, 4, 5, 6, 7, 8, 9, or 10 cycles.

Also provided herein are methods of treating of treating myelofibrosis in a subject, said method comprising:

administering a JAK2 inhibitor to the subject;

administering an activin receptor type IIB (ActRIIB) ligand trap to the subject;

wherein the concomitantly administration of the activin receptor type IIB (ActRIIB) ligand trap is at an effective dose that the JAK2 inhibitor dose is not adjusted due to adverse effects. In certain embodiments, the JAK2 inhibitor dose is not decreased. In certain embodiments, the adverse effect is anemia. In one embodiment, the adverse effect is decrease in red blood cell count. In one embodiment, the adverse effect is increase in units of red blood cell (RBC) transfusions.

Also provided herein are methods of treating of treating myelofibrosis in a subject, said method comprising:

administering a JAK2 inhibitor to the subject at an initial dose;

administering an activin receptor type IIB (ActRIIB) ligand trap to the subject; and

maintaining the initial dose of the JAK2 inhibitor for an extended period of time.

In certain embodiments, the concomitantly administration of the activin receptor type IIB (ActRIIB) ligand trap is at an effective dose to reduce or mitigate adverse effects. In certain embodiments, the JAK2 inhibitor dose is not decreased. In certain embodiments, the adverse effect is anemia. In some embodiments, the initial dose of the JAK2 inhibitor is the recommended starting dose. In one embodiment, when the JAK2 inhibitor is ruxolitinib, the initial dose is 20 mg twice daily. In another embodiment, when the JAK2 inhibitor is ruxolitinib, the initial dose is 15 mg twice daily. In one embodiment, when the JAK2 inhibitor is fedratinib, the initial dose is 400 mg once daily. In another embodiment, when the JAK2 inhibitor is fedratinib, the initial dose is 300 mg once daily. In yet another embodiment, when the JAK2 inhibitor is fedratinib, the initial dose is 200 mg once daily. In certain embodiments, the JAK2 inhibitor is administered at least 75%, 80%, 85%, 90%, or 95% of the recommended starting dose. In certain embodiments, the initial dose is an adjusted dose when concomitant strong CYP3A4 inhibitor is administered. In certain embodiments, the recommended starting dose is an adjusted recommended starting dose when concomitant strong CYP3A4 inhibitor is administered. In certain embodiments, the JAK2 inhibitor is administered at the initial dose for at least 1, 2, 3, 4, 5, 6, 7, 8, 9, 10, 11, or 12 months. In certain embodiments, the JAK2 inhibitor is administered at the initial dose for at least 1, 2, 3, 4, 5, 6, 7, 8, 9, or 10 years.

Also provided herein are methods for treating anemia in a subject with an activin receptor type IIB (ActRIIB) ligand trap, said method comprising: administering an activin receptor type IIB (ActRIIB) ligand trap to the subject in need of treatment, wherein the subject is receiving a concomitant administration of a JAK2 inhibitor for the treatment of myelofibrosis. In certain embodiments, the concomitant treatment of JAK2 inhibitor is administered at the normal dose for an extended period of time. In certain embodiments, the JAK2 inhibitor is administered at the normal dose for at least 1, 2, 3, 4, 5, 6, 7, 8, 9, 10, 11, or 12 months. In certain embodiments, the JAK2 inhibitor is administered at the normal dose for at least 1, 2, 3, 4, 5, 6, 7, 8, 9, or 10 years.

In one embodiment of the above methods in this subsection 5.9.1, the activin receptor type IIB (ActRIIB) ligand trap is an activin receptor type IIB (ActRIIB) ligand trap disclosed herein.

### 5.10 Routes of Administration

In certain embodiments, the ActRIIB ligand trap is administered via injection. In certain embodiments, the ActRIIB ligand trap is administered subcutaneously. In certain embodiments, the ActRIIB ligand trap is administered once every 3 weeks. In specific embodiments, the ActRIIB ligand trap is a polypeptide comprising the amino acid sequence of SEQ ID NO:11. In specific embodiments, the ActRIIB ligand trap is a polypeptide comprising an amino acid sequence consisting of the amino acid sequence of SEQ ID NO:11. In specific embodiments, the ActRIIB ligand trap is a polypeptide consisting of the amino acid sequence set forth in SEQ ID NO:11. In a preferred embodiment, the ActRIIB ligand trap is luspatercept.

The route of administration of fedratinib or pharmaceutically acceptable salt or hydrate thereof, is independent of the route of administration of the ActRIIB ligand trap provided herein. In one preferred embodiment, fedratinib is administered orally. In another embodiment, fedratinib is administered intravenously. In yet another embodiment, fedratinib is administered subcutaneously.

### 6. EXAMPLES

### 6.1 Example 1: A Phase 3B, Multicenter, Single-Arm, Open-Label Efficacy and Safety Study of Fedratinib with Concomitant Luspatercept for Treatment of Anemia of in Subject with Myeloproliferative Neoplasm (MPN) Associated Myelofibrosis

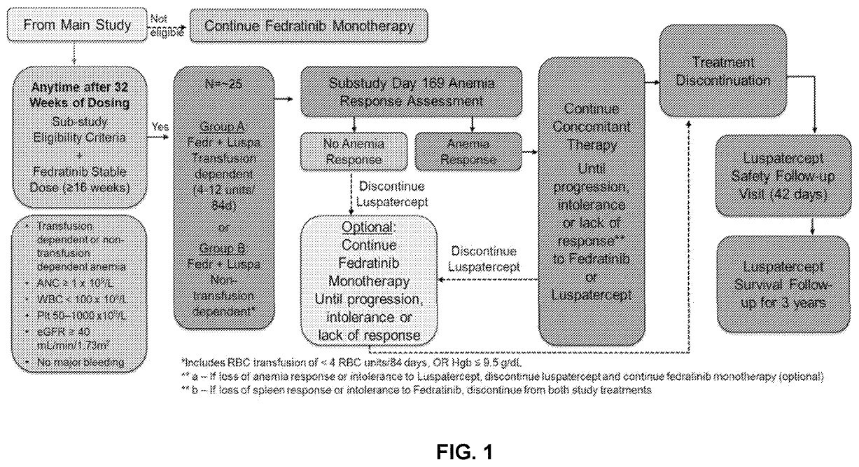

This example relates to a sub-study of a Phase 3B, multicenter, single-arm, open-label study. The main study is to evaluate efficacy and safety of fedratinib in subjects with Dynamic International Prognostic Scoring System (DIPSS) intermediate or high-risk MPN-associated MF (including PMF, post-PV and post-ET MF). This sub-study is to evaluate the safety, tolerability, and efficacy of luspatercept, an ActRIIB ligand trap, when administered concomitantly with fedratinib in subjects with MPN-associated MF and anemia. This sub-study begins no earlier than 32 weeks after the initial fedratinib dose. This sub-study is divided into a Sub-study Screening Period (during the main study), a Sub-study Treatment Period (consisting of a Sub-study Treatment Phase and a Sub-study Week 24 (Sub-study Day 169) Anemia Response Assessment), followed by a Sub-study Post-treatment Follow-up Period (consisting of a 42-Day Safety Follow-up and a Sub-study Long-Term Follow-Up Period). The overall study design is described in FIG. 1.

This sub-study primarily evaluates the safety and tolerability of luspatercept when administered concomitantly with fedratinib, as well as evaluate efficacy in two groups of patients with MPN-associated MF and anemia: 1) Group A (transfusion dependent): subjects with an RBC baseline transfusion burden of 4-12 RBC units/84 days, with no interval of >6 weeks (42 days) without an RBC transfusion; 2) Group B (non-transfusion dependent): subjects with an RBC baseline transfusion burden of <4 RBC units/84 days OR anemia (Hgb levels of ≤9.5 g/dL recorded on ≥3 different days, including the day of dosing, in the 84-day period immediately up to the Sub-study Cycle 1, Day 1 (C1D1) date.

Subjects who meet the transfusion or anemia criteria and have received fedratinib for at least 32 weeks during the main study while on a stable dose of fedratinib for the immediate 16 weeks prior to sub-study Cycle 1, Day 1 (C1D1) are eligible for participation in the sub-study.

An interim safety monitoring review is performed after approximately 6 subjects complete at least 2 cycles treatment with luspatercept in the sub-study by safety monitoring. Safety is evaluated using all available safety data on an ongoing basis for this concomitant treatment (including at least the 6 subjects that received 2 cycles) and will be compared to the monotherapy safety data for each treatment. If no major toxicity (defined as unacceptable or unmanageable toxicity) is observed, enrollment will continue and complete for the remaining subjects.

Subjects receive concomitant treatment with luspatercept until the Anemia Response Assessment on Sub-study Week 24 (Sub-study Day 169) and may continue concomitant treatment as long as they continue to derive clinical benefit (anemia, spleen, or symptom response) as assessed by the Investigator unless they develop progressive disease (according to the International Working Group-Myeloproliferative Neoplasms Research and Treatment (IWG-MRT) 2013 criteria) or unacceptable toxicities, withdraw consent, or meet any other criteria for treatment discontinuation. Upon assessment, the subject may: 1) continue with concomitant treatment, 2) discontinue luspatercept and continue fedratinib monotherapy, or 3) discontinue both study treatments.

Due to the different dosing schedule and table of events, the visits in the sub-study occur differently from the main study. Planned visits in the sub-study are conducted in 3-week cycles based on the established luspatercept dosing schedule. Once a subject enters the Sub-study, the subject continues following the sub-study schedule of events.

Approximately 25 subjects from previously treated with ruxolitinib and with PMF, post-PV MF or post-ET MF and with anemia from the main study are enrolled into the optional sub-study. An interim safety monitoring review is performed after ˜6 subjects complete at least 2 cycles of concomitant treatment.

### 6.1.1 Study Designs and Study Periods

### (a) Screening Period

Screening into the sub-study is conducted while the subjects are receiving treatment on the main study. Any subject with anemia who has had at least 32 weeks of fedratinib treatment would be eligible and may screen for the sub-study at any timepoint. All subjects who sign consent for the sub-study will proceed through a maximum 28-day Screening Period.

### (b) Enrollment

Eligible subjects are allocated to either Group A or Group B based on the inclusion criteria met.
Group A: Transfusion dependent (4-12 RBC units/84 days) anemia
Group B: Non-transfusion dependent (receiving <4 RBC units/84 days OR anemia only with a Hgb≤9.5 g/dL) anemia

Ineligible subjects continue in the main study, receiving fedratinib only and completing cycle visits according to the main Study.

### (c) Treatment Period

The starting dose of luspatercept is 1.33 mg/kg administered as a subcutaneous injection Anemia Response Assessment (Week 24 (Sub-study Day 169))

All subjects enrolled remain on therapy at least through the end of Sub-study Week 24 (Sub-study Day 168). An Anemia Response Assessment to assess clinical benefit (anemia, spleen, or symptom response) is performed to determine if a subject may continue concomitant treatment.

Subjects may remain on concomitant treatment following the Anemia Response Assessment on Sub-study Week 24 (Sub-study Day 169) if they continue to derive clinical benefit and the subject does not meet any treatment discontinuation criteria (i.e., unacceptable toxicities, progressive disease, subject withdrawal). The clinical benefit assessment occurs every cycle after the Anemia Response Assessment.

### 6.1.2 Inclusion Criteria

Approximately 25 subjects from previously treated with ruxolitinib and with PMF, post-PV MF or post-ET MF and with anemia (as defined below) from the main study are enrolled into this sub-study.

Subjects satisfy the following criteria for the main study to be enrolled in the study:
1. Subject is at least 18 years of age at the time of signing the informed consent form (ICF);
2. Subject has an Eastern Cooperative Oncology Group (ECOG) Performance Score (PS) of 0, 1 or 2;
3. Subject has diagnosis of primary myelofibrosis (PMF) according to the 2016 World Health Organization (WHO) criteria, or diagnosis of post-ET or post-PV myelofibrosis according to the IWG-MRT 2007 criteria, confirmed by the most recent local pathology report;
4. Subject has a DIPSS Risk score of Intermediate or High;
5. Subject has a measurable splenomegaly during the screening period as demonstrated by spleen volume of ≥450 cm³ by MRI or CT-scan assessment or by palpable spleen measuring ≥5 cm below the left costal margin;
6. Subject has been previously exposed to ruxolitinib while diagnosed with MF (PMF, post-ET MF or post-PV MF), and must meet at least one of the following criteria (a or b):
   a. Treatment with ruxolitinib for ≥3 months;
   b. Treatment with ruxolitinib for ≥28 days complicated by any of the following:
      i. Development of a red blood cell transfusion requirement (at least 2 units/month for 2 months) or
      ii. Grade ≥3 AEs of thrombocytopenia, anemia, hematoma, and/or hemorrhage while on treatment with ruxolitinib;
7. Subject must have treatment-related toxicities from prior therapy resolved to Grade 1 or pretreatment baseline before start of last therapy prior to fedratinib treatment;
8. Subject must understand and voluntarily sign an ICF prior to any study-related assessments/procedures being conducted;
9. Subject is willing and able to adhere to the study visit schedule and other protocol requirements;
10. A female of childbearing potential (FCBP) must:
   a. Have two negative pregnancy tests as verified by the Investigator prior to starting study therapy. She must agree to ongoing pregnancy testing during the course of the study, and after end of study therapy. This applies even if the subject practices true abstinence* from heterosexual contact;
   b. Either commit to true abstinence from heterosexual contact (which must be reviewed on a monthly basis and source documented) or agree to use, and be able to comply with highly effective contraception without interruption, −14 days prior to starting investigational product, during the study therapy (including dose interruptions), and for 30 days after discontinuation of study therapy;
11. Male subjects must:
   a. Practice true abstinence (which must be reviewed on a monthly basis) or agree to use a condom during sexual contact with a pregnant female or a female of childbearing potential while participating in the study, during dose interruptions and for at least 30 days following investigational product discontinuation, or longer if required for each compound and/or by local regulations, even if he has undergone a successful vasectomy.

In addition to the main study's criteria, upon enrollment in the sub-study, all subjects must meet the following inclusion criteria:
1. Subject must understand and voluntarily sign an optional sub-study ICF prior to any sub-study-related assessments/procedures being conducted
2. Subject must have been taking fedratinib for at least 32 weeks (˜8 cycles)
3. Subject must be on a stable dose of fedratinib for at least 16 weeks (˜4 cycles) [no dose level changes] in the time immediately up to the projected date of enrollment (SC1D1)
4. Subject has anemia defined as either:
   a. Group A—Transfusion dependent (TD) anemia
      RBC-transfusion frequency*: 4 to 12 RBC units/84 days immediately up to the SC1D1 date (Sub-study Cycle 1 Day 1), with no interval of >6 weeks (42 days) without an RBC transfusion.
      Subjects must have a Hgb value of <11.5 g/dL on SC1D1 prior to luspatercept administration.
   b. Group B—Non-transfusion dependent (NTD) anemia
      RBC-transfusion frequency: <4 RBC units/84 days immediately up to the SC1D1 date; or
      At least 3 Hgb levels of ≤9.5 g/dL recorded on ≥3 different days, including the day of dosing, in the 84-day period immediately up to Sub-study C1D1 date. There must be ≥14 days in between each Hgb measurement. No subjects with an interval ≥42 days between hemoglobin measurements will be enrolled.
   “Baseline” is defined as the 84-day rolling period (3 cycles at 28 days each) prior to Sub-study Cycle 1 Day 1. Any transfusions given either at a Hgb≤7 or for a Hgb≤9.5 g/dL with symptoms will be counted towards baseline transfusion needs.
   Transfusions given only for bleeding or infections will not be counted towards eligibility baseline transfusion requirements.
5. Subject has an Eastern Cooperative Oncology Group (ECOG) performance score of ≤2

The subject is willing and able to adhere to the study visit schedule and other protocol requirements.

### 6.1.3 Exclusion Criteria

1. Any of the following laboratory abnormalities:
   a. Platelets <50,000/μL
   b. Absolute neutrophil count (ANC)<1.0×109/L
   c. White blood count (WBC)>100×109/L
   d. Myeloblasts ≥5% in peripheral blood
   e. Estimated glomerular filtration rate <30 mL/min/1.73 m2 (as per the Modification of Diet in Renal Disease [MDRD] formula)
   f. Serum amylase or lipase >1.5×ULN (upper limit of normal)
   g. Aspartate aminotransferase (AST) or alanine aminotransferase (ALT)>3×ULN
   h. Total bilirubin >1.5×ULN, subject's total bilirubin between 1.5-3.0×ULN are eligible if the direct bilirubin fraction is <25% of the total bilirubin
2. Subject is pregnant or lactating female
3. Subject with previous splenectomy
4. Subject with previous or planned hematopoietic cell transplant
5. Subject with prior history of encephalopathy, including Wernicke's
6. Subject with signs or symptoms of encephalopathy including Wernicke's (e.g., severe ataxia, ocular paralysis or cerebellar signs)
7. Subject with thiamine deficiency, defined as thiamine levels in whole blood below normal range according to institutional standard and not corrected prior to enrollment on the study
8. Subject with concomitant treatment with or use of pharmaceutical, herbal agents or food known to be strong or moderate inducers of Cytochrome P450 3A4 (CYP3A4), or dual CYP2C19 and CYP3A4 inhibitors
9. Subject on any chemotherapy, immunomodulatory drug therapy (e.g., thalidomide, interferon-alpha), anagrelide, immunosuppressive therapy, systemic corticosteroids >10 mg/day prednisone or equivalent. Subjects who have had prior exposure to hydroxyurea (e.g., Hydrea) in the past may be enrolled into the study as long as it has not been administered within 14 days prior to the start of fedratinib treatment
10. Subject has received ruxolitinib within 14 days prior to the start of fedratinib
11. Subject on treatment with myeloid growth factor (e.g., granulocyte-colony stimulating factor [G-CSF]) within 14 days prior to the start of fedratinib treatment
12. Subject with previous exposure to Janus kinase (JAK) inhibitor(s) for more than 1 cycle other than ruxolitinib treatment
13. Subject on treatment with aspirin with doses >150 mg daily
14. Subject with major surgery within 28 days before starting fedratinib treatment
15. Subject with diagnosis of chronic liver disease (e.g., chronic alcoholic liver disease, autoimmune hepatitis, sclerosing cholangitis, primary biliary cirrhosis, hemochromatosis, non-alcoholic steatohepatitis)
16. Subject with prior malignancy other than the disease under study unless the subject has not required treatment for the malignancy for at least 3 years prior to enrollment. However, subject with the following history/concurrent conditions provided successfully treated may enroll: non-invasive skin cancer, in situ cervical cancer, carcinoma in situ of the breast, incidental histologic finding of prostate cancer (T1a or T1b using the tumor, nodes, metastasis [TNM] clinical staging system), or is free of disease and on hormonal treatment only.
17. Subject with uncontrolled congestive heart failure (New York Heart Association Classification 3 or 4)
18. Subject with known human immunodeficiency virus (HIV), known active infectious Hepatitis B (HepB), and/or known active infectious Hepatitis C (HepC)
19. Subject with serious active infection
20. Subject with presence of any significant gastric or other disorder that would inhibit absorption of oral medication
21. Subject is unable to swallow capsule
22. Subject has any significant medical condition, laboratory abnormality, or psychiatric illness that would prevent the subject from participating in the study
23. Subject has any condition including the presence of laboratory abnormalities, which places the subject at unacceptable risk if he/she were to participate in the study
24. Subject has any condition that confounds the ability to interpret data from the study
25. Subject with participation in any study of an investigational agent (drug, biologic, device) within 30 days prior to start of fedratinib treatment
26. Subject with life expectancy of less than 6 months.

### 6.1.4 Sub-Study Exclusion Criteria

1. Subject with anemia from causes other than MPN-associated MF or JAK2 inhibitor therapy (e.g., iron deficiency, vitamin B12 and/or folate deficiencies, autoimmune or hemolytic anemia, infection, or any type of known clinically significant bleeding or sequestration).
2. Subject with any of the following laboratory abnormalities at SC1D1:
   a. Neutrophils <1×109/L
   b. White blood count (WBC)>100×109/L
   c. Platelets <50×109/L or >1000×109/L
   d. Peripheral blood myeloblasts >5%.
   e. Estimated glomerular filtration rate <40 mL/min/1.73 m2 (via the 4-variable modification of diet in renal disease [MDRD] formula)
   f. Aspartate aminotransferase (AST) or alanine transaminase (ALT)>3.0× upper limit of normal (ULN)
   g. Direct bilirubin ≥2×ULN (higher levels are acceptable if these can be attributed to active red blood cell precursor destruction within the bone marrow (i.e., ineffective erythropoiesis))
3. Subject with diastolic blood pressure ≥90 mmHg or systolic blood pressure ≥140 mmHg before SC1D1 despite appropriate treatment.
4. Subject with prior malignancy other than the disease under study unless the subject has not required treatment for the malignancy for at least 3 years prior to enrollment. However, subject with the following history/concurrent conditions is allowed:
   a. Basal or squamous cell carcinoma of the skin
   b. Carcinoma in situ of the cervix
   c. Carcinoma in situ of the breast
   d. Incidental histologic finding of prostate cancer (T1a or T1b using the tumor, nodes, metastasis [TNM] clinical staging system)
5. Subject with stroke, deep venous thrombosis, pulmonary or arterial embolism within 6 months immediately up to SC1D1.
6. Subject with major surgery within 2 months up to the enrollment date. Subject must have completely recovered from any previous surgery immediately up to the enrollment date.
7. Subject with inadequately controlled heart disease and/or have a known left ventricular ejection fraction <35%.
8. Subject with uncontrolled systemic fungal, bacterial, or viral infection (defined as ongoing signs/symptoms related to the infection without improvement despite appropriate antibiotics, antiviral therapy, and/or other treatment).
9. Subject with prior therapy of luspatercept or sotatercept.
10. Subject with history of severe allergic or anaphylactic reactions or hypersensitivity to recombinant proteins or excipients in the investigational products (see luspatercept IB).
11. Subject with a major bleeding event (defined as symptomatic bleeding in a critical area or organ and/or bleeding causing a decrease in Hgb of ≥2 g/dL or leading to transfusion of ≥2 units of packed red cells) in the last 6 months prior to enrollment.
12. Subject use of erythropoietin-stimulating agents (ESA)≤56 days prior to SC1D1.

### 6.1.5 Treatment Administration and Schedule

Subjects continue to receive fedratinib at the same dose level administered during the main study. In addition, subjects also receive luspatercept on Day 1 of each 21-day treatment cycle through at least Day 168 following the sub-study Cycle 1, Day 1 (SC1D1) date unless the subject experiences progression to AML, unacceptable toxicities, withdraws consent, or meets any other treatment discontinuation criteria prior to the Day 169 Response Assessment. Treatment cycles are 21 days in duration.

### (a) Administration of Fedratinib

The fedratinib dose is 400 mg/day PO (4×100 mg capsules) to be self-administered orally once daily continuously on an outpatient basis, preferably together with an evening meal, the same time each day. However, fedratinib may be taken with or without regard to food. In case a dose is missed, the next dose is taken the following day at the same time of day as previously taken before the dose was missed.

A flexible dose modification regimen may be employed to minimize drug toxicity for individual subjects, with possible daily doses of 100 mg, 200 mg, 300 mg, or 400 mg. For administrative purposes cycles are defined as 4-week (28-day) periods. Subjects may continue treatment with fedratinib until unacceptable toxicity, lack of therapeutic effect, progression of disease, or until consent is withdrawn.

### (b) Administration of Luspatercept

Luspatercept is administered on Day 1 of every 21-day cycle, at an initial dose level of 1.33 mg/kg. Doses may be titrated up starting in Cycle 3 as described herein. Luspatercept is administered as a subcutaneous injection to subjects. Subjects have their Hgb, WBC, blood myeloblast percentage, and blood pressure assessed prior to each administration.

Luspatercept starting dose with dose increases and reductions are listed in Table 1.

TABLE 1
Luspatercept Starting Dose Level with Dose
Reductions and Dose Titrations

| | | | Starting | 1^{st} Dose |
|---|---|---|---|---|
| 3^{rd} Dose | 2^{nd} Dose | 1^{st} Dose | Dose | Titration |
| Reduction | Reduction | Reduction | Level | Increase |
| 0.6 mg/kg | 0.8 mg/kg | 1.0 mg/kg | 1.33 mg/kg | 1.75 mg/kg |

### (c) Luspatercept Dose Titration

As soon as Sub-study Cycle 3 Day 1 and assessed by the Investigator prior to every subsequent treatment cycle, subjects may have the dose level increased from the starting dose level of 1.33 mg/kg up to a maximum of 1.75 mg/kg during the Sub-study Treatment Phase.

If the subject has two of the most recent prior treatment cycles assessed at the same dose level and if the subject has not met any protocol dose delay and/or reduction criteria in the 2 most prior treatment cycles, they may be eligible for a dose titration. The dose level may be increased by 1 dose level if 1 or more of the following criteria are met:
Subject has ≥1 RBC transfusion event (for pretransfusion Hgb of ≤9.5 g/dL) during the 2 most recent prior treatment cycles (˜6 weeks)
Hgb decrease of ≥1 g/dL is observed in a transfusion-free period of approximately 6 weeks and this decrease is not preceded by an RBC transfusion (the Hgb decrease occurs ≥14 days after the last RBC transfusion)
Hgb value is not exceeding 1 g/dL increase from the baseline mean pretransfusion Hgb value

The dose level should be titrated individually for each subject.

### 6.1.6 Endpoints

The primary objective is to evaluate the safety and tolerability of fedratinib administered concomitantly with luspatercept. With a sample size of 25, the standard error for the estimation of response rate and adverse event rates will be no more than 0.1. For the secondary endpoint of anemia response related to modified 8-week Hematological Improvement—Erythroid Response (HI-E), the estimated response rate is at least 50%, based on an anticipated rate of 12-week mean ≥1.5 g/dL increase in hemoglobin or ≥50% RBC transfusion reduction (of at least 4 units) over any 12-week period.

Primary and secondary endpoints of this sub-study are listed in Table 2 below. Any data collected during the sub-study may be used for the main study analysis including, but not limited to, spleen volume, spleen size by palpation, MFSAF v4.0 (Myelofibrosis Symptom Assessment Form), and thiamine (and Mini-Mental State Exam [MMSE]) monitoring.

TABLE 2
Study Endpoints

| Endpoint | Name | Description | Assessment Timeframe |
|---|---|---|---|
| Primary | Safety and | Incidence and severity of all Grade | From enrollment |
| | tolerability of | AEs and Grade 3-4 AEs per NCI | (SC1D1) up until 42 |
| | luspatercept | CTCAE | days post last dose of |
| | administered | | luspatercept or 30 days |
| | concomitantly with | | post last dose of |
| | fedratinib | | fedratinib (whichever is |
| | | | later); |
| | | | For fedratinib or |
| | | | luspatercept related AEs, |
| | | | anytime until the last |
| | | | study visit |
| Endpoint | Name | Description | Assessment Timeframe |
| Secondary | Anemia Response | Hgb increase by ≥ 1.5 g/dL | Any consecutive |
| | related to modified | OR | “rolling” 56-day period |
| | Hematological | Reduction of units of RBC | starting from SC1D1 |
| | Improvement- | transfusions by an absolute | through and including |
| | Erythroid Response | number of at least 4 RBC | Sub-study Week 24 (Day |
| | (HI-E) | transfusions/56 days compared | 169) |
| | (Groups A and B) | with baseline (8 weeks). | Also from SC1D1 |
| | | | through EOT |
| | Anemia Response | Proportion of RBC-transfusion | Starting from SC1D1 |
| | related to Reduction | dependent subjects who reduce | through and including |
| | in Transfusion | their transfusion burden by ≥ 50% | Sub-study Week 24 (Day |
| | Burden (Group A) | and by ≥ 4 units/12 weeks from | 169) |
| | | baseline over any consecutive 12- | Also from SC1D1 |
| | | week period | through EOT |
| | Anemia Response | Proportion of subjects achieving a | Any consecutive |
| | related to Mean | mean ≥1.5 g/dL hemoglobin | “rolling” 84-day period |
| | Hemoglobin Increase | increase from baseline over any | starting from SC1D1 |
| | (Group B) | consecutive 84-day period without | through and including |
| | | an RBC transfusion | Sub-study Week 24 (Day |
| | | | 169) |
| | | | Also from SC1D1 |
| | | | through EOT |
| | Anemia response | Proportion of subjects who | Any consecutive |
| | related to RBC- | become RBC-transfusion free over | “rolling” 84-day period |
| | transfusion | any consecutive 84-day period | starting from SC1D1 |
| | independence | | through and including |
| | (Group A and B) | | Sub-study Week 24 (Day |
| | | | 169); |
| | | | Also from SC1D1 |
| | | | through EOT |
| | Duration of anemia | Maximum duration of anemia | From SC1D1 through |
| | response | response in each of the endpoints | EOT |
| | | and groups | |
| | Mean change in | Change in serum erythropoietin | From SC1D1 through |
| | serum erythropoietin | | EOT and including Week |
| | from baseline | | 24 (Day 169) |
| | | | From SC1D1 through |
| | | | EOT |
| | Mean change in | Change in serum ferritin | From SC1D1 through |
| | serum ferritin from | | EOT and including Week |
| | baseline | | 24 (Day 169) |
| | | | From SC1D1 through |
| | | | EOT |

### 6.1.7 RBC Transfusions

Red blood cell-transfusion practices based on Hgb levels and symptoms should not change for an individual subject during the Screening Period and the Treatment Phase.

For any RBC transfusions received during the study (at either the study site or an outside institution), the Hgb value just prior to transfusion should be collected, along with several other parameters (i.e., number of units transfused, volume transfused, date of transfusion).

Each subject has a “pretransfusion hemoglobin threshold” for requiring transfusion during the study, which is determined based on transfusion history. The baseline pretransfusion hemoglobin threshold will be the mean of all documented pretransfusion hemoglobin values during the 12 weeks prior to Sub-study Cycle 1 Day 1. During treatment, if the pretransfusion hemoglobin level is increased by ≥1 g/dL (at the time of a next anticipated transfusion event) compared to the pretransfusion hemoglobin threshold for that subject, the transfusion should be delayed by a minimum of 7 days and/or the number of units transfused should be reduced by 1 or more RBC units.

For the purpose of this study, RBC or whole blood transfusions are regarded as equivalent.

### 7. DESCRIPTION OF THE SEQUENCES

TABLE 3
Sequence Information.

| SEQ | | |
|---|---|---|
| ID | | |
| NO. | DESCRIPTION | SEQUENCE |
| 1 | human ActRIIB soluble | ETRECIYYNANWELERTNQSGLERCEGEQDKRLHCYAS |
| | (extracellular), processed | WRNSSGTIELVKKGCWDDDFNCYDRQECVATEENPQVY |
| | polypeptide sequence | FCCCEGNFCNERFTHLPEAGGPEVTYEPPP |
| | with the N-terminal 6 | |
| | amino acids of the EC | |
| | domain deleted and the | |
| | C-terminal 4 amino acids | |
| | of the EC domain deleted | |
| | (amino acids 25-130 of | |
| | SEQ ID NO: 14) and with | |
| | an L79D mutation | |
| 2 | human ActRIIB | MTAPWVALALLWGSLWPGSGRGEAETRECIYYNANWE |
| | precursor protein | LERTNQSGLERCEGEQDKRLHCYASWANSSGTIELVKK |
| | sequence (A64) | GCWLDDFNCYDRQECVATEENPQVYFCCCEGNFCNERF |
| | | THLPEAGGPEVTYEPPPTAPTLLTVLAYSLLPIGGLSLIVL |
| | | LAFWMYRHRKPPYGHVDIHEDPGPPPPSPLVGLKPLQLL |
| | | EIKARGRFGCVWKAQLMNDFVAVKIFPLQDKQSWQSER |
| | | EIFSTPGMKHENLLQFIAAEKRGSNLEVELWLITAFHDKG |
| | | SLTDYLKGNIITWNELCHVAETMSRGLSYLHEDVPWCR |
| | | GEGHKPSIAHRDFKSKNVLLKSDLTAVLADFGLAVRFEP |
| | | GKPPGDTHGQVGTRRYMAPEVLEGAINFQRDAFLRIDM |
| | | YAMGLVLWELVSRCKAADGPVDEYMLPFEEEIGQHPSL |
| | | EELQEVVVHKKMRPTIKDHWLKHPGLAQLCVTIEECWD |
| | | HDAEARLSAGCVEERVSLIRRSVNGTTSDCLVSLVTSVT |
| | | NVDLPPKESSI |
| 3 | human ActRIIB soluble | SGRGEAETRECIYYNANWELERTNQSGLERCEGEQDKR |
| | (extracellular), processed | LHCYASWANSSGTIELVKKGCWLDDFNCYDRQECVATE |
| | polypeptide sequence | ENPQVYFCCCEGNFCNERFTHLPEAGGPEVTYEPPPTAPT |
| | (amino acids 19-134 of | |
| | SEQ ID NO: 2) | |
| 4 | human ActRIIB soluble | SGRGEAETRECIYYNANWELERTNQSGLERCEGEQDKR |
| | (extracellular), processed | LHCYASWANSSGTIELVKKGCWLDDFNCYDRQECVATE |
| | polypeptide sequence | ENPQVYFCCCEGNFCNERFTHLPEA |
| | with the C-terminal 15 | |
| | amino acids deleted | |
| | (amino acids 19-119 of | |
| | SEQ ID NO: 2) | |
| 5 | nucleic acid sequence | ATGACGGCGCCCTGGGTGGCCCTCGCCCTCCTCTGGG |
| | encoding a human | GATCGCTGTGGCCCGGCTCTGGGCGTGGGGAGGCTGA |
| | ActRIIB (A64) precursor | GACACGGGAGTGCATCTACTACAACGCCAACTGGGAG |
| | protein | CTGGAGCGCACCAACCAGAGCGGCCTGGAGCGCTGCG |
| | | AAGGCGAGCAGGACAAGCGGCTGCACTGCTACGCCTC |
| | | CTGGGCCAACAGCTCTGGCACCATCGAGCTCGTGAAG |
| | | AAGGGCTGCTGGCTAGATGACTTCAACTGCTACGATA |
| | | GGCAGGAGTGTGTGGCCACTGAGGAGAACCCCCAGGT |
| | | GTACTTCTGCTGCTGTGAAGGCAACTTCTGCAACGAGC |
| | | GCTTCACTCATTTGCCAGAGGCTGGGGGCCCGGAAGT |
| | | CACGTACGAGCCACCCCCGACAGCCCCCACCCTGCTC |
| | | ACGGTGCTGGCCTACTCACTGCTGCCCATCGGGGGCCT |
| | | TTCCCTCATCGTCCTGCTGGCCTTTTGGATGTACCGGC |
| | | ATCGCAAGCCCCCCTACGGTCATGTGGACATCCATGA |
| | | GGACCCTGGGCCTCCACCACCATCCCCTCTGGTGGGCC |
| | | TGAAGCCACTGCAGCTGCTGGAGATCAAGGCTCGGGG |
| | | GCGCTTTGGCTGTGTCTGGAAGGCCCAGCTCATGAAT |
| | | GACTTTGTAGCTGTCAAGATCTTCCCACTCCAGGACAA |
| | | GCAGTCGTGGCAGAGTGAACGGGAGATCTTCAGCACA |
| | | CCTGGCATGAAGCACGAGAACCTGCTACAGTTCATTG |
| | | CTGCCGAGAAGCGAGGCTCCAACCTCGAAGTAGAGCT |
| | | GTGGCTCATCACGGCCTTCCATGACAAGGGCTCCCTCA |
| | | CGGATTACCTCAAGGGGAACATCATCACATGGAACGA |
| | | ACTGTGTCATGTAGCAGAGACGATGTCACGAGGCCTC |
| | | TCATACCTGCATGAGGATGTGCCCTGGTGCCGTGGCG |
| | | AGGGCCACAAGCCGTCTATTGCCCACAGGGACTTTAA |
| | | AAGTAAGAATGTATTGCTGAAGAGCGACCTCACAGCC |
| | | GTGCTGGCTGACTTTGGCTTGGCTGTTCGATTTGAGCC |
| | | AGGGAAACCTCCAGGGGACACCCACGGACAGGTAGG |
| | | CACGAGACGGTACATGGCTCCTGAGGTGCTCGAGGGA |
| | | GCCATCAACTTCCAGAGAGATGCCTTCCTGCGCATTGA |
| | | CATGTATGCCATGGGGTTGGTGCTGTGGGAGCTTGTGT |
| | | CTCGCTGCAAGGCTGCAGACGGACCCGTGGATGAGTA |
| | | CATGCTGCCCTTTGAGGAAGAGATTGGCCAGCACCCT |
| | | TCGTTGGAGGAGCTGCAGGAGGTGGTGGTGCACAAGA |
| | | AGATGAGGCCCACCATTAAAGATCACTGGTTGAAACA |
| | | CCCGGGCCTGGCCCAGCTTTGTGTGACCATCGAGGAG |
| | | TGCTGGGACCATGATGCAGAGGCTCGCTTGTCCGCGG |
| | | GCTGTGTGGAGGAGCGGGTGTCCCTGATTCGGAGGTC |
| | | GGTCAACGGCACTACCTCGGACTGTCTCGTTTCCCTGG |
| | | TGACCTCTGTCACCAATGTGGACCTGCCCCCTAAAGA |
| | | GTCAAGCATCTAA |
| 6 | fusion protein comprising | SGRGEAETRECIYYNANWELERTNQSGLERCEGEQDKR |
| | a soluble extracellular | LHCYASWANSSGTIELVKKGCWLDDFNCYDRQECVATE |
| | domain of ActRIIB | ENPQVYFCCCEGNFCNERFTHLPEAGGPEVTYEPPPTAPT |
| | (A64; SEQ ID NO: 3) | GGGTHTCPPCPAPELLGGPSVFLFPPKPKDTLMISRTPEV |
| | fused to an Fc domain | TCVVVDVSHEDPEVKFNWYVDGVEVHNAKTKPREEQY |
| | | NSTYRVVSVLTVLHQDWLNGKEYKCKVSNKALPVPIEK |
| | | TISKAKGQPREPQVYTLPPSREEMTKNQVSLTCLVKGFY |
| | | PSDIAVEWESNGQPENNYKTTPPVLDSDGSFFLYSKLTV |
| | | DKSRWQQGNVFSCSVMHEALHNHYTQKSLSLSPGK |
| 7 | fusion protein comprising | SGRGEAETRECIYYNANWELERTNQSGLERCEGEQDKR |
| | a soluble extracellular | LHCYASWANSSGTIELVKKGCWLDDFNCYDRQECVATE |
| | domain of ActRIIB | ENPQVYFCCCEGNFCNERFTHLPEAGGGTHTCPPCPAPE |
| | (A64) with the C- | LLGGPSVFLFPPKPKDTLMISRTPEVTCVVVDVSHEDPEV |
| | terminal 15 amino acids | KFNWYVDGVEVHNAKTKPREEQYNSTYRVVSVLTVLH |
| | deleted (SEQ ID NO: 4) | QDWLNGKEYKCKVSNKALPVPIEKTISKAKGQPREPQV |
| | fused to an Fc domain | YTLPPSREEMTKNQVSLTCLVKGFYPSDIAVEWESNGQP |
| | | ENNYKTTPPVLDSDGSFFLYSKLTVDKSRWQQGNVFSCS |
| | | VMHEALHNHYTQKSLSLSPGK |
| 8 | human ActRIIB soluble | ETRECIYYNANWELERTNQSGLERCEGEQDKRLHCYAS |
| | (extracellular), processed | WRNSSGTIELVKKGCWDDDFNCYDRQECVATEENPQVY |
| | polypeptide sequence | FCCCEGNFCNERFTHLPEAGGPEVTYEPP |
| | with the N-terminal 6 | |
| | amino acids of the EC | |
| | domain deleted and the | |
| | C-terminal 5 amino acids | |
| | of the EC domain deleted | |
| | (amino acids 25-129 of | |
| | SEQ ID NO: 14) and with | |
| | an L79D mutation | |
| 9 | human ActRIIB soluble | ETRECIYYNANWELERTNQSGLERCEGEQDKRLHCYAS |
| | (extracellular), processed | WRNSSGTIELVKKGCWDDDFNCYDRQECVATEENPQVY |
| | polypeptide sequence | FCCCEGNFCNERFTHLPEAGGPEVTYEPPPT |
| | with the N-terminal 6 | |
| | amino acids of the EC | |
| | domain deleted and the | |
| | C-terminal 3 amino acids | |
| | of the EC domain deleted | |
| | (amino acids 25-131 of | |
| | SEQ ID NO: 14) and with | |
| | an L79D mutation | |
| 10 | Unprocessed ActRIIB-Fc | MDAMKRGLCCVLLLCGAVFVSPGAAETRECIYYNANW |
| | fusion protein with the | ELERTNQSGLERCEGEQDKRLHCYASWRNSSGTIELVKK |
| | N-terminal 6 amino acids | GCWDDDFNCYDRQECVATEENPQVYFCCCEGNFCNERF |
| | of the EC domain deleted | THLPEAGGPEVTYEPPPTGGGTHTCPPCPAPELLGGPSVF |
| | and the C-terminal 3 | LFPPKPKDTLMISRTPEVTCVVVDVSHEDPEVKFNWYVD |
| | amino acids of the EC | GVEVHNAKTKPREEQYNSTYRVVSVLTVLHQDWLNGK |
| | domain deleted (amino | EYKCKVSNKALPAPIEKTISKAKGQPREPQVYTLPPSREE |
| | acids 25-131 of SEQ ID | MTKNQVSLTCLVKGFYPSDIAVEWESNGQPENNYKTTPP |
| | NO: 14) and with an | VLDSDGSFFLYSKLTVDKSRWQQGNVFSCSVMHEALHN |
| | L79D mutation and with | HYTQKSLSLSPGK |
| | TPA leader sequence | |
| 11 | Processed ActRIIB-Fc | ETRECIYYNANWELERTNQSGLERCEGEQDKRLHCYAS |
| | fusion protein with the | WRNSSGTIELVKKGCWDDDFNCYDRQECVATEENPQVY |
| | N-terminal 6 amino acids | FCCCEGNFCNERFTHLPEAGGPEVTYEPPPTGGGTHTCPP |
| | of the EC domain deleted | CPAPELLGGPSVFLFPPKPKDTLMISRTPEVTCVVVDVSH |
| | and the C-terminal 3 | EDPEVI<FNWYVDGVEVHNAI<TI<PREEQYNSTYRVVSV |
| | amino acids of the EC | LTVLHQDWLNGKEYKCKVSNKALPAPIEKTISKAKGQPR |
| | domain deleted (amino | EPQVYTLPPSREEMTKNQVSLTCLVKGFYPSDIAVEWES |
| | acids 25-131 of SEQ ID | NGQPENNYKTTPPVLDSDGSFFLYSKLTVDKSRWQQGN |
| | NO: 14) and with an | VFSCSVMHEALHNHYTQKSLSLSPGK |
| | L79D mutation | |
| | (Luspatercept) | |
| 12 | human ActRIIB soluble | GRGEAETRECIYYNANWELERTNQSGLERCEGEQDKRL |
| | (extracellular), processed | HCYASWANSSGTIELVKKGCWLDDFNCYDRQECVATEE |
| | polypeptide sequence | NPQVYFCCCEGNFCNERFTHLPEAGGPEVTYEPPPTAPT |
| | (amino acids 20-134 of | |
| | SEQ ID NO: 2) | |
| 13 | human ActRIIB soluble | GRGEAETRECIYYNANWELERTNQSGLERCEGEQDKRL |
| | (extracellular), processed | HCYASWANSSGTIELVKKGCWLDDFNCYDRQECVATEE |
| | polypeptide sequence | NPQVYFCCCEGNFCNERFTHLPEA |
| | with the C-terminal 15 | |
| | amino acids deleted | |
| | (amino acids 20-119 of | |
| | SEQ ID NO: 2) | |
| 14 | human ActRIIB | MTAPWVALALLWGSLWPGSGRGEAETRECIYYNANWE |
| | precursor protein | LERTNQSGLERCEGEQDKRLHCYASWRNSSGTIELVKKG |
| | sequence (R64) | CWLDDFNCYDRQECVATEENPQVYFCCCEGNFCNERFT |
| | | HLPEAGGPEVTYEPPPTAPTLLTVLAYSLLPIGGLSLIVLL |
| | | AFWMYRHRKPPYGHVDIHEDPGPPPPSPLVGLKPLQLLEI |
| | | KARGRFGCVWKAQLMNDFVAVKIFPLQDKQSWQSEREI |
| | | FSTPGMKHENLLQFIAAEKRGSNLEVELWLITAFHDKGS |
| | | LTDYLKGNIITWNELCHVAETMSRGLSYLHEDVPWCRG |
| | | EGHKPSIAHRDFKSKNVLLKSDLTAVLADFGLAVRFEPG |
| | | KPPGDTHGQVGTRRYMAPEVLEGAINFQRDAFLRIDMY |
| | | AMGLVLWELVSRCKAADGPVDEYMLPFEEEIGQHPSLE |
| | | ELQEVVVHKKMRPTIKDHWLKHPGLAQLCVTIEECWDH |
| | | DAEARLSAGCVEERVSLIRRSVNGTTSDCLVSLVTSVTN |
| | | VDLPPKESSI |
| 15 | human ActRIIB soluble | SGRGEAETRECIYYNANWELERTNQSGLERCEGEQDKR |
| | (extracellular), processed | LHCYASWRNSSGTIELVKKGCWLDDFNCYDRQECVATE |
| | polypeptide sequence | ENPQVYFCCCEGNFCNERFTHLPEAGGPEVTYEPPPTAPT |
| | (amino acids 19-134 of | |
| | SEQ ID NO: 14) | |
| 16 | human ActRIIB soluble | SGRGEAETRECIYYNANWELERTNQSGLERCEGEQDKR |
| | (extracellular), processed | LHCYASWRNSSGTIELVKKGCWLDDFNCYDRQECVATE |
| | polypeptide sequence | ENPQVYFCCCEGNFCNERFTHLPEA |
| | with the C-terminal 15 | |
| | amino acids deleted | |
| | (amino acids 19-119 of | |
| | SEQ ID NO: 14) | |
| 17 | human ActRIIB soluble | GRGEAETRECIYYNANWELERTNQSGLERCEGEQDKRL |
| | (extracellular), processed | HCYASWRNSSGTIELVKKGCWLDDFNCYDRQECVATEE |
| | polypeptide sequence | NPQVYFCCCEGNFCNERFTHLPEAGGPEVTYEPPPTAPT |
| | (amino acids 20-134 of | |
| | SEQ ID NO: 14) | |
| 18 | human ActRIIB soluble | GRGEAETRECIYYNANWELERTNQSGLERCEGEQDKRL |
| | (extracellular), processed | HCYASWRNSSGTIELVKKGCWLDDFNCYDRQECVATEE |
| | polypeptide sequence | NPQVYFCCCEGNFCNERFTHLPEA |
| | with the C-terminal 15 | |
| | amino acids deleted | |
| | (amino acids 20-119 of | |
| | SEQ ID NO: 14) | |
| 19 | human ActRIIB soluble | ETRECIYYNANWELERTNQSGLERCEGEQDKRLHCYAS |
| | (extracellular), processed | WANSSGTIELVKKGCWDDDFNCYDRQECVATEENPQV |
| | polypeptide sequence | YFCCCEGNFCNERFTHLPEAGGPEVTYEPPPT |
| | with the N-terminal 6 | |
| | amino acids of the EC | |
| | domain deleted and the | |
| | C-terminal 3 amino acids | |
| | of the EC domain deleted | |
| | (amino acids 25-131 of | |
| | SEQ ID NO: 2) and with | |
| | an L79D mutation | |
| 20 | Unprocessed ActRIIB-Fc | MDAMKRGLCCVLLLCGAVFVSPGAAETRECIYYNANW |
| | fusion protein with the | ELERTNQSGLERCEGEQDKRLHCYASWANSSGTIELVKK |
| | N-terminal 6 amino acids | GCWDDDFNCYDRQECVATEENPQVYFCCCEGNFCNERF |
| | of the EC domain deleted | THLPEAGGPEVTYEPPPTGGGTHTCPPCPAPELLGGPSVF |
| | and the C-terminal 3 | LFPPKPKDTLMISRTPEVTCVVVDVSHEDPEVKFNWYVD |
| | amino acids of the EC | GVEVHNAKTKPREEQYNSTYRVVSVLTVLHQDWLNGK |
| | domain deleted (amino | EYKCKVSNKALPAPIEKTISKAKGQPREPQVYTLPPSREE |
| | acids 25-131 of SEQ ID | MTKNQVSLTCLVKGFYPSDIAVEWESNGQPENNYKTTPP |
| | NO: 2) and with an L79D | VLDSDGSFFLYSKLTVDKSRWQQGNVFSCSVMHEALHN |
| | mutation and with TPA | HYTQKSLSLSPGK |
| | leader sequence | |
| 21 | Processed ActRIIB-Fc | ETRECIYYNANWELERTNQSGLERCEGEQDKRLHCYAS |
| | fusion protein with the | WANSSGTIELVKKGCWDDDFNCYDRQECVATEENPQV |
| | N-terminal 6 amino acids | YFCCCEGNFCNERFTHLPEAGGPEVTYEPPPTGGGTHTC |
| | of the EC domain deleted | PPCPAPELLGGPSVFLFPPKPKDTLMISRTPEVTCVVVDV |
| | and the C-terminal 3 | SHEDPEVKFNWYVDGVEVHNAKTKPREEQYNSTYRVVS |
| | amino acids of the EC | VLTVLHQDWLNGKEYKCKVSNKALPAPIEKTISKAKGQ |
| | domain deleted (amino | PREPQVYTLPPSREEMTKNQVSLTCLVKGFYPSDIAVEW |
| | acids 25-131 of SEQ ID | ESNGQPENNYKTTPPVLDSDGSFFLYSKLTVDKSRWQQG |
| | NO: 2) and with an L79D | NVFSCSVMHEALHNHYTQKSLSLSPGK |
| | mutation | |
| 22 | human ActRIIB soluble | GRGEAETRECIYYNANWELERTNQSGLERCEGEQDKRL |
| | (extracellular), processed | HCYASWRNSSGTIELVKKGCWDDDFNCYDRQECVATEE |
| | polypeptide sequence | NPQVYFCCCEGNFCNERFTHLPEAGGPEVTYEPPPTAPT |
| | (amino acids 20-134 of | |
| | SEQ ID NO: 14) with | |
| | L79D mutation | |
| 23 | human ActRIIB soluble | GRGEAETRECIYYNANWELERTNQSGLERCEGEQDKRL |
| | (extracellular), processed | HCYASWANSSGTIELVKKGCWDDDFNCYDRQECVATEE |
| | polypeptide sequence | NPQVYFCCCEGNFCNERFTHLPEAGGPEVTYEPPPTAPT |
| | (amino acids 20-134 of | |
| | SEQ ID NO: 2) with | |
| | L79D mutation | |
| 24 | human ActRIIB soluble | GRGEAETRECIYYNANWELERTNQSGLERCEGEQDKRL |
| | (extracellular), processed | HCYASWRNSSGTIELVKKGCWDDDFNCYDRQECVATEE |
| | polypeptide sequence | NPQVYFCCCEGNFCNERFTHLPEAGGPEVTYEPPPTAPT |
| | (amino acids 20-134 of | GGGTHTCPPCPAPELLGGPSVFLFPPKPKDTLMISRTPEV |
| | SEQ ID NO: 14) with | TCVVVDVSHEDPEVKFNWYVDGVEVHNAKTKPREEQY |
| | L79D mutation fused to | NSTYRVVSVLTVLHQDWLNGKEYKCKVSNKALPAPIEK |
| | an Fc domain with a | TISKAKGQPREPQVYTLPPSREEMTKNQVSLTCLVKGFY |
| | GGG linker | PSDIAVEWESNGQPENNYKTTPPVLDSDGSFFLYSKLTV |
| | | DKSRWQQGNVFSCSVMHEALHNHYTQKSLSLSPGK |
| 25 | human ActRIIB soluble | GRGEAETRECIYYNANWELERTNQSGLERCEGEQDKRL |
| | (extracellular), processed | HCYASWANSSGTIELVKKGCWDDDFNCYDRQECVATEE |
| | polypeptide sequence | NPQVYFCCCEGNFCNERFTHLPEAGGPEVTYEPPPTAPT |
| | (amino acids 20-134 of | GGGTHTCPPCPAPELLGGPSVFLFPPKPKDTLMISRTPEV |
| | SEQ ID NO: 2) with | TCVVVDVSHEDPEVKFNWYVDGVEVHNAKTKPREEQY |
| | L79D mutation fused to | NSTYRVVSVLTVLHQDWLNGKEYKCKVSNKALPAPIEK |
| | an Fc domain | TISKAKGQPREPQVYTLPPSREEMTKNQVSLTCLVKGFY |
| | | PSDIAVEWESNGQPENNYKTTPPVLDSDGSFFLYSKLTV |
| | | DKSRWQQGNVFSCSVMHEALHNHYTQKSLSLSPGK |
| 26 | human ActRIIB soluble | MDAMKRGLCCVLLLCGAVFVSPGASGRGEAETRECIYY |
| | (extracellular), processed | NANWELERTNQSGLERCEGEQDKRLHCYASWRNSSGTI |
| | polypeptide sequence | ELVKKGCWDDDFNCYDRQECVATEENPQVYFCCCEGN |
| | (amino acids 20-134 of | FCNERFTHLPEAGGPEVTYEPPPTAPTGGGTHTCPPCPAP |
| | SEQ ID NO: 14) with | ELLGGPSVFLFPPKPKDTLMISRTPEVTCVVVDVSHEDPE |
| | L79D mutation fused to | VKFNWYVDGVEVHNAKTKPREEQYNSTYRVVSVLTVL |
| | an Fc domain and with | HQDWLNGKEYKCKVSNKALPAPIEKTISKAKGQPREPQ |
| | TPA leader sequence | VYTLPPSREEMTKNQVSLTCLVKGFYPSDIAVEWESNGQ |
| | | PENNYKTTPPVLDSDGSFFLYSKLTVDKSRWQQGNVFSC |
| | | SVMHEALHNHYTQKSLSLSPGK |
| 27 | human ActRIIB soluble | MDAMKRGLCCVLLLCGAVFVSPGASGRGEAETRECIYY |
| | (extracellular), processed | NANWELERTNQSGLERCEGEQDKRLHCYASWANSSGTI |
| | polypeptide sequence | ELVKKGCWDDDFNCYDRQECVATEENPQVYFCCCEGN |
| | (amino acids 20-134 of | FCNERFTHLPEAGGPEVTYEPPPTAPTGGGTHTCPPCPAP |
| | SEQ ID NO: 2) with | ELLGGPSVFLFPPKPKDTLMISRTPEVTCVVVDVSHEDPE |
| | L79D mutation fused to | VKFNWYVDGVEVHNAKTKPREEQYNSTYRVVSVLTVL |
| | an Fc domain and with | HQDWLNGKEYKCKVSNKALPAPIEKTISKAKGQPREPQ |
| | TPA leader sequence | VYTLPPSREEMTKNQVSLTCLVKGFYPSDIAVEWESNGQ |
| | | PENNYKTTPPVLDSDGSFFLYSKLTVDKSRWQQGNVFSC |
| | | SVMHEALHNHYTQKSLSLSPGK |
| 28 | human ActRIIB soluble | GRGEAETRECIYYNANWELERTNQSGLERCEGEQDKRL |
| | (extracellular), processed | HCYASWRNSSGTIELVKKGCWLDDFNCYDRQECVATEE |
| | polypeptide sequence | NPQVYFCCCEGNFCNERFTHLPEAGGPEGPWASTTIPSG |
| | having a variant C- | GPEATAAAGDQGSGALWLCLEGPAHE |
| | terminal sequence | |
| | (disclosed in | |
| | WO2007/053775) | |
| 29 | human ActRIIB soluble | GRGEAETRECIYYNANWELERTNQSGLERCEGEQDKRL |
| | (extracellular), processed | HCYASWRNSSGTIELVKKGCWDDDFNCYDRQECVATEE |
| | polypeptide sequence | NPQVYFCCCEGNFCNERFTHLPEAGGPEGPWASTTIPSG |
| | having a variant C- | GPEATAAAGDQGSGALWLCLEGPAHE |
| | terminal sequence | |
| | (disclosed in | |
| | WO2007/053775) having | |
| | an L79D mutation | |
| 30 | human ActRIIB soluble | GRGEAETRECIYYNANWELERTNQSGLERCEGEQDKRL |
| | (extracellular), processed | HCYASWRNSSGTIELVKKGCWDDDFNCYDRQECVATEE |
| | polypeptide sequence | NPQVYFCCCEGNFCNERFTHLPEAGGPEGPWASTTIPSG |
| | having a variant C- | GPEATAAAGDQGSGALWLCLEGPAHETGGGTHTCPPCP |
| | terminal sequence | APELLGGPSVFLFPPKPKDTLMISRTPEVTCVVVDVSHED |
| | (disclosed in | PEVKFNWYVDGVEVHNAKTKPREEQYNSTYRVVSVLT |
| | WO2007/053775) having | VLHQDWLNGKEYKCKVSNKALPAPIEKTISKAKGQPREP |
| | an L79D mutation fused | QVYTLPPSREEMTKNQVSLTCLVKGFYPSDIAVEWESNG |
| | to an Fc domain with a | QPENNYKTTPPVLDSDGSFFLYSKLTVDKSRWQQGNVFS |
| | TGGG linker | CSVMHEALHNHYTQKSLSLSPGK |
| 31 | Nucleic Acid Sequence | ATGGATGCAATGAAGAGAGGGCTCTGCTGTGTGCTGC |
| | Encoding SEQ ID NO: 10 | TGCTGTGTGGAGCAGTCTTCGTTTCGCCCGGCGCCGCC |
| | | GAAACCCGCGAATGTATTTATTACAATGCTAATTGGG |
| | | AACTCGAACGGACGAACCAATCCGGGCTCGAACGGTG |
| | | TGAGGGGGAACAGGATAAACGCCTCCATTGCTATGCG |
| | | TCGTGGAGGAACTCCTCCGGGACGATTGAACTGGTCA |
| | | AGAAAGGGTGCTGGGACGACGATTTCAATTGTTATGA |
| | | CCGCCAGGAATGTGTCGCGACCGAAGAGAATCCGCAG |
| | | GTCTATTTCTGTTGTTGCGAGGGGAATTTCTGTAATGA |
| | | ACGGTTTACCCACCTCCCCGAAGCCGGCGGGCCCGAG |
| | | GTGACCTATGAACCCCCGCCCACCGGTGGTGGAACTC |
| | | ACACATGCCCACCGTGCCCAGCACCTGAACTCCTGGG |
| | | GGGACCGTCAGTCTTCCTCTTCCCCCCAAAACCCAAGG |
| | | ACACCCTCATGATCTCCCGGACCCCTGAGGTCACATGC |
| | | GTGGTGGTGGACGTGAGCCACGAAGACCCTGAGGTCA |
| | | AGTTCAACTGGTACGTGGACGGCGTGGAGGTGCATAA |
| | | TGCCAAGACAAAGCCGCGGGAGGAGCAGTACAACAG |
| | | CACGTACCGTGTGGTCAGCGTCCTCACCGTCCTGCACC |
| | | AGGACTGGCTGAATGGCAAGGAGTACAAGTGCAAGGT |
| | | CTCCAACAAAGCCCTCCCAGCCCCCATCGAGAAAACC |
| | | ATCTCCAAAGCCAAAGGGCAGCCCCGAGAACCACAGG |
| | | TGTACACCCTGCCCCCATCCCGGGAGGAGATGACCAA |
| | | GAACCAGGTCAGCCTGACCTGCCTGGTCAAAGGCTTC |
| | | TATCCCAGCGACATCGCCGTGGAGTGGGAGAGCAATG |
| | | GGCAGCCGGAGAACAACTACAAGACCACGCCTCCCGT |
| | | GCTGGACTCCGACGGCTCCTTCTTCCTCTATAGCAAGC |
| | | TCACCGTGGACAAGAGCAGGTGGCAGCAGGGGAACG |
| | | TCTTCTCATGCTCCGTGATGCATGAGGCTCTGCACAAC |
| | | CACTACACGCAGAAGAGCCTCTCCCTGTCCCCGGGTA |
| | | AATGA |
| 32 | fusion protein comprising | SGRGEAETRECIYYNANWELERTNQSGLERCEGEQDKR |
| | a soluble extracellular | LHCYASWRNSSGTIELVKKGCWLDDFNCYDRQECVATE |
| | domain of ActRIIB (R64; | ENPQVYFCCCEGNFCNERFTHLPEAGGPEVTYEPPPTAPT |
| | SEQ ID NO: 15) fused to | GGGTHTCPPCPAPELLGGPSVFLFPPKPKDTLMISRTPEV |
| | an Fc domain | TCVVVDVSHEDPEVKFNWYVDGVEVHNAKTKPREEQY |
| | | NSTYRVVSVLTVLHQDWLNGKEYKCKVSNKALPVPIEK |
| | | TISKAKGQPREPQVYTLPPSREEMTKNQVSLTCLVKGFY |
| | | PSDIAVEWESNGQPENNYKTTPPVLDSDGSFFLYSKLTV |
| | | DKSRWQQGNVFSCSVMHEALHNHYTQKSLSLSPGK |
| 33 | fusion protein comprising | SGRGEAETRECIYYNANWELERTNQSGLERCEGEQDKR |
| | a soluble extracellular | LHCYASWRNSSGTIELVKKGCWLDDFNCYDRQECVATE |
| | domain of ActRIIB | ENPQVYFCCCEGNFCNERFTHLPEAGGGTHTCPPCPAPE |
| | (R64) with the C- | LLGGPSVFLFPPKPKDTLMISRTPEVTCVVVDVSHEDPEV |
| | terminal 15 amino acids | KFNWYVDGVEVHNAKTKPREEQYNSTYRVVSVLTVLH |
| | deleted (SEQ ID NO: 16) | QDWLNGKEYKCKVSNKALPVPIEKTISKAKGQPREPQV |
| | fused to an Fc domain | YTLPPSREEMTKNQVSLTCLVKGFYPSDIAVEWESNGQP |
| | | ENNYKTTPPVLDSDGSFFLYSKLTVDKSRWQQGNVFSCS |
| | | VMHEALHNHYTQKSLSLSPGK |
| 34 | Nucleic acid sequence | ATGGATGCAATGAAGAGA GGGCTCTGCTGTGTGCTG |
| | that encodes SEQ ID | CTGCTGTGTGGAGCAGTC TTCGTTTCGCCCGGCGCC |
| | NO: 11 and tissue | GCCGAAACCCGCGAATGT ATTTATTACAATGCTAAT |
| | plasminogen activator | TGGGAACTCGAACGGACG AACCAATCCGGGCTCGAA |
| | (TPA signal peptide | CGGTGTGAGGGGGAACAG GATAAACGCCTCCATTGC |
| | sequence) | TATGCGTCGTGGAGGAAC TCCTCCGGGACGATTGAA |
| | (TPA signal peptide | CTGGTCAAGAAAGGGTGC TGGGACGACGATTTCAAT |
| | sequence underlined and | TGTTATGACCGCCAGGAA TGTGTCGCGACCGAAGAG |
| | shown in bold). | AATCCGCAGGTCTATTTC TGTTGTTGCGAGGGGAAT |
| | | TTCTGTAATGAACGGTTT ACCCACCTCCCCGAAGCC |
| | | GGCGGGCCCGAGGTGACC TATGAACCCCCGCCCACC |
| | | GGTGGTGGAACTCACACA TGCCCACCGTGCCCAGCA |
| | | CCTGAACTCCTGGGGGGA CCGTCAGTCTTCCTCTTC |
| | | CCCCCAAAACCCAAGGAC ACCCTCATGATCTCCCGG |
| | | ACCCCTGAGGTCACATGC GTGGTGGTGGACGTGAGC |
| | | CACGAAGACCCTGAGGTC AAGTTCAACTGGTACGTG |
| | | GACGGCGTGGAGGTGCAT AATGCCAAGACAAAGCCG |
| | | CGGGAGGAGCAGTACAAC AGCACGTACCGTGTGGTC |
| | | AGCGTCCTCACCGTCCTG CACCAGGACTGGCTGAAT |
| | | GGCAAGGAGTACAAGTGC AAGGTCTCCAACAAAGCC |
| | | CTCCCAGCCCCCATCGAG AAAACCATCTCCAAAGCC |
| | | AAAGGGCAGCCCCGAGAA CCACAGGTGTACACCCTG |
| | | CCCCCATCCCGGGAGGAG ATGACCAAGAACCAGGTC |
| | | AGCCTGACCTGCCTGGTC AAAGGCTTCTATCCCAGC |
| | | GACATCGCCGTGGAGTGG GAGAGCAATGGGCAGCCG |
| | | GAGAACAACTACAAGACC ACGCCTCCCGTGCTGGAG |
| | | TCCGACGGCTCCTTCTTC CTCTATAGCAAGCTCACC |
| | | GTGGACAAGAGCAGGTGG CAGCAGGGGAACGTCTTC |
| | | TCATGCTCCGTGATGCAT GAGGCTCTGCACAACCAC |
| | | TACACGCAGAAGAGCCTC TCCCTGTCTCCGGGTAAA |
| | | TGA |

### 8. EQUIVALENTS

Although the invention is described in detail with reference to specific embodiments thereof, it will be understood that variations which are functionally equivalent are within the scope of this invention. Indeed, various modifications of the invention in addition to those shown and described herein will become apparent to those skilled in the art from the foregoing description and accompanying drawings. Such modifications are intended to fall within the scope of the appended claims. Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference in their entireties.

What is claimed:

1. A method for treating anemia in a subject in need thereof, comprising:
administering to a subject an activin receptor type IIB (ActRIIB) ligand trap; and
administering to the subject fedratinib or a pharmaceutically acceptable salt and/or hydrate thereof.

2. A method for treating anemia in a subject in need thereof, comprising:
(a) taking a first measurement of hemoglobin (Hgb) level in a subject;
(b) administering to the subject an initial dose of an ActRIIB ligand trap;
(c) administering to the subject fedratinib or a pharmaceutically acceptable salt and/or hydrate thereof;
(d) taking a second measurement of hemoglobin (Hgb) level in the subject at the end of a first period of time after the administration of the initial dose of an ActRIIB ligand trap; and
(e) administering to the subject a subsequent dose of the ActRIIB ligand trap based on the second measurement of hemoglobin (Hgb) level as compared to the first measurement of hemoglobin (Hgb) level, or based on number of red blood cell transfusion that the subject received during the first period of time.

3. The method of claim 1 or 2, wherein the ActRIIB ligand trap and fedratinib or pharmaceutically acceptable salt or hydrate thereof are concomitantly administered.

4. The method of any one of claims 1 to 3, wherein the co-administration of the ActRIIB ligand trap and fedratinib or pharmaceutically acceptable salt or hydrate thereof is pharmaceutically effective to treat anemia.

5. The method of any one of claims 1 to 4, wherein the subject is a subject is diagnosed with myelofibrosis.

6. The method of claim 5, wherein the myelofibrosis is a myeloproliferative neoplasms (MPN)-associated myelofibrosis.

7. The method of claim 5, wherein the myelofibrosis is intermediate or High-Risk Primary Myelofibrosis (PMF), Post-Polycythemia Vera Myelofibrosis (post-PV MF), or Post-Essential Thrombocythemia Myelofibrosis (post-ET MF).

8. The method of claim 7, wherein the myelofibrosis is intermediate or high-risk primary myelofibrosis (PMF).

9. The method of claim 7, wherein the myelofibrosis is intermediate or high-risk post-polycythemia vera myelofibrosis (post-PV MF).

10. The method of claim 7, wherein the myelofibrosis is intermediate or high-risk post-essential thrombocythemia myelofibrosis (post-ET MF).

11. The method of any one of claims 1 to 10, wherein the subject is a human.

12. The method of any one of claims 1 to 11, wherein the anemia is associated with myeloproliferative neoplasms (MPN)-associated myelofibrosis.

13. The method of any one of claims 1 to 11, wherein the subject is red blood cell (RBC) transfusion dependent or non-transfusion dependent.

14. The method of any one of claims 1 to 13, wherein the subject is RBC transfusion dependent.

15. The method of claim 14, wherein the subject has received 4 to 12 RBC units in RBC transfusion within 84 days prior to administering of the ActRIIB ligand trap.

16. The method of claim 14, wherein the subject has a hemoglobin (Hgb) level of less than 11.5 g/dL without red blood cell (RBC) transfusion.

17. The method of any one of claims 1 to 13, wherein the subject is non-transfusion dependent.

18. The method of claim 17, wherein the subject has received less than 4 red blood cell (RBC) units in RBC transfusion within 84 days prior to administering of the ActRIIB ligand trap.

19. The method of claim 17, wherein the subject has a hemoglobin (Hgb) level of less than 9.5 g/dL.

20. The method of any one of claims 1 to 19, wherein the subject has treated with fedratinib for at least 8 weeks, at least 16 weeks, at least 24 weeks, at least 32 weeks, or at least 40 weeks prior to administration of the initial dose of the ActRIIB ligand trap and concomitantly administration of fedratinib or pharmaceutically acceptable salt or hydrate thereof.

21. The method of any one of claims 1 to 20, wherein the subject has been previously treated with ruxolitinib.

22. The method of any one of claims 1 to 21, wherein the fedratinib or pharmaceutically acceptable salt or hydrate thereof is administered daily.

23. The method of any one of claims 1 to 22, wherein the fedratinib or pharmaceutically acceptable salt or hydrate thereof is administered orally.

24. The method of any one of claims 1 to 23, wherein the fedratinib or pharmaceutically acceptable salt or hydrate thereof is administered at a dosage of 400 mg/day.

25. The method of any one of claims 1 to 24, wherein the ActRIIB ligand trap is administered once at the beginning of every treatment cycle, wherein each cycle is 21 days.

26. The method of any one of claims 1 to 25, wherein the ActRIIB ligand trap is administered to the subject for at least 1, 2, 3, 4, 5, 6, 7, 8, 9, or 10 cycles.

27. The method of any one of claims 1 to 26, wherein the ActRIIB ligand trap is administered to the subject subcutaneously.

28. The method of any one of claims 1 to 27, wherein the pharmaceutically effective amount of the ActRIIB ligand trap administered is 0.6 mg/kg, 0.8 mg/kg, 1 mg/kg, 1.33 mg/kg, or 1.75 mg/kg.

29. The method of claim 28, wherein the pharmaceutically effective amount of the ActRIIB ligand trap administered is 1.33 mg/kg.

30. The method of any one of claims 2 to 27, wherein the first measurement of hemoglobin (Hgb) level is taken prior to the administration of the initial dose of the ActRIIB ligand trap.

31. The method of any one of claims 2 to 27, wherein the first measurement of hemoglobin (Hgb) level is taken concurrently with the administration of the initial dose of the ActRIIB ligand trap, or taken about 3 weeks, 6 weeks, 9 weeks, 12 weeks, 15 weeks, 18 weeks, 21 weeks, or 24 weeks after the administration of the initial dose of the ActRIIB ligand trap.

32. The method of any one of claims 2 to 31, wherein the second measurement of hemoglobin (Hgb) level is taken about 3 weeks, 6 weeks, 9 weeks, 12 weeks, 15 weeks, 18 weeks, 21 weeks, 24 weeks, 7 months, 8 months, 9 months, 10 months, 11 months, or 12 months after the initial dose of the ActRIIB ligand trap is administered to the subject.

33. The method of any one of claims 2 to 32, wherein the first period of time is 1 day, 2 days, 3 days, 4 days, 5 days, 6 days, 1 week, 2 weeks, 3 weeks, 4 weeks, 5 weeks, 6 weeks, 7 weeks, or 8 weeks.

34. The method of any one of claim 33, wherein the first period of time is 6 weeks.

35. The method of any one of claims 2 to 34, wherein the initial dose of the ActRIIB ligand trap is 1.33 mg/kg.

36. The method of any one of claims 2 to 35, wherein the subsequent dose of the ActRIIB ligand trap is 0.6 mg/kg, 0.8 mg/kg, 1.0 mg/kg, 1.33 mg/kg, or 1.75 mg/kg.

37. The method of any one of claims 2 to 35, wherein the subsequent dose of the ActRIIB ligand trap is 1.33 mg/kg.

38. The method of any one of claims 2 to 35, wherein when the second measurement of Hgb level is 2 g/dL or more higher than the first measurement of Hgb level, the subsequent dose of the ActRIIB ligand trap is lower than the initial dose of the ActRIIB ligand trap.

39. The method of any one of claims 2 to 35, wherein when the subject has one or more RBC transfusion during the first period of time, or the second measurement of Hgb level is between 0 to about 1 g/dL higher than the first measurement of Hgb level, or the first measurement of Hgb level decreases 1 g/dL or more in a transfusion-free period of approximately 6 weeks, the subsequent dose of the ActRIIB ligand trap is higher than the initial dose of the ActRIIB ligand trap.

40. The method of any one of claims 2 to 35, wherein the subsequent dose of the ActRIIB ligand trap is the same as the initial dose of the ActRIIB ligand trap.

41. The method of any one of claims 1 to 40, further comprising:
grading hematological, hepatic, non-hematological, or gastrointestinal event in the subject as Grade 1, 2, 3, 4, or 5 according to the National Cancer Institute (NCI) Common Terminology Criteria for Adverse Events (CTCAE); and
administering a subsequent dose of fedratinib or pharmaceutically acceptable salt or hydrate thereof.

42. The method of claim 41, wherein the subsequent dose of fedratinib or pharmaceutically acceptable salt or hydrate thereof is 300 mg/day, 200 mg/day, or 100 mg/day.

43. The method of any one of claims 1 to 42, wherein the ActRIIB ligand trap is a humanized fusion-protein consisting of the extracellular domain of ActRIIB and the human IgG1 Fc domain.

44. The method of any one of claims 1 to 42, wherein the ActRIIB ligand trap is a fusion-protein comprising the extracellular domain of ActRIIB and the human IgG1 Fc domain.

45. The method of any one of claims 1 to 42, wherein the ActRIIB ligand trap is a polypeptide comprising an amino acid sequence selected from the group consisting of:
(a) 90% identical to SEQ ID NO:3;
(b) 95% identical to SEQ ID NO:3;
(c) 98% identical to SEQ ID NO:3;
(d) SEQ ID NO:4;
(e) 90% identical to SEQ ID NO:6;
(f) 95% identical to SEQ ID NO:6;
(g) 98% identical to SEQ ID NO:6;
(h) SEQ ID NO:7;
(i) 90% identical to SEQ ID NO:7;
(j) 95% identical to SEQ ID NO:7;
(k) 98% identical to SEQ ID NO:7;
(l) SEQ ID NO:8;
(m) 90% identical to SEQ ID NO:11;
(n) 95% identical to SEQ ID NO: 11;
(o) 98% identical to SEQ ID NO: 11; and
(p) SEQ ID NO:11.

46. The method of any one of claims 1 to 42, wherein the ActRIIB ligand trap is a polypeptide comprising an amino acid sequence selected from the group consisting of:
(a) 90% identical to SEQ ID NO:11;
(b) 95% identical to SEQ ID NO: 11;
(c) 98% identical to SEQ ID NO: 11; and
(d) SEQ ID NO:11.

47. The method of any one of claims 1 to 42, wherein the ActRIIB ligand trap is a polypeptide comprising the amino acid sequence of SEQ ID NO:11.

48. The method of any one of claims 1 to 42, wherein the ActRIIB ligand trap is a polypeptide is encoded by the nucleotide sequence of SEQ ID NO:34 or a degenerate version of SEQ ID NO:34.

49. The method of any one of claims 1 to 47, wherein the method increases the hemoglobin (Hgb) level in the subject by at least 0.5 g/dL, at least 1.0 g/dL, at least 1.5 g/dL, at least 2.0 g/dL, or at least 2.5 g/dL.

50. The method of claim 49, wherein the method increases the hemoglobin (Hgb) level in the subject by at least 1.5 g/dL.

51. The method of any one of claims 1 to 47, wherein the method reduces at least 2, at least 3, at least 4, at least 5, at least 6, at least 7, at least 8, at least 9, or at least 10 units of red blood cell (RBC) transfusions received by the subject within a period of 56 days.

52. The method of claim 51, wherein the method reduces at least 4 units of RBC transfusions received by the subject within a period of 56 days.

53. The method of any one of claims 1 to 47, wherein the method reduces at least 2, at least 3, at least 4, at least 5, at least 6, at least 7, at least 8, at least 9, or at least 10 units of red blood cell (RBC) transfusions received by the subject within a period of 84 days.

54. The method of any one of claims 1 to 47, wherein the method increases the hemoglobin (Hgb) level in the subject by at least 1.5 g/dL over a consecutive 84-day period.

55. The method of any one of claims 1 to 47, wherein the subject becomes red blood cell (RBC) transfusion free over a consecutive period of 84 days.

56. The method of any one of claims 1 to 55, wherein the method increases hemoglobin (HGB) levels in the subject to levels equal to or about 1%, 2%, 3%, 4%, 5%, 6%, 7%, 8%, 9% 10%, 20%, 25%, 30%, 40%, 50%, 60%, 70%, 75%, 80%, 90%, 100%, 200%, or 500% greater than HGB levels in the subject prior to said treating.

57. The method of any one of claims 1 to 56, wherein the method increases hematocrit (HCT) levels in the subject to levels equal to or about 1%, 2%, 3%, 4%, 5%, 6%, 7%, 8%, 9%, 10%, 20%, 25%, 30%, 40%, 50%, 60%, 70%, 75%, 80%, 90%, 100%, 200%, or 500% greater than HCT levels in the subject prior to said treating.

58. The method of any one of claims 1 to 57, wherein the method reduces mean corpuscular volume (MCV) levels in the subject to levels equal to or about 1%, 2%, 3%, 4%, 5%, 6%, 7%, 8%, 9%, 10%, 20%, 25%, 30%, 40%, 50%, 60%, 70%, 75%, 80%, 90%, or 100%, less than MCV levels in the subject prior to said treating.

59. The method of any one of claims 1 to 58, wherein the method increases cellular hemoglobin concentration (CHC) levels in the subject to levels equal to or about 1%, 2%, 3%, 4%, 5%, 6%, 7%, 8%, 9%, 10%, 20%, 25%, 30%, 40%, 50%, 60%, 70%, 75%, 80%, 90%, 100%, 200%, or 500% greater than CHC levels in the subject prior to said treating

60. The method of any one of claims 1 to 59, wherein the method reduces red blood cell distribution width (RDW) levels in the subject to levels equal to or about 1%, 2%, 3%, 4%, 5%, 6%, 7%, 8%, 9%, 10%, 20%, 25%, 30%, 40%, 50%, 60%, 70%, 75%, 80%, 90%, or 100%, less than the RDW levels in the subject prior to said treating

61. The method of any one of claims 1 to 60, wherein the levels of reticulocytes in the subject remain in the range equal to or about 0.1%, 0.5%, 1%, 2%, 3%, 4%, 5%, 6%, 7%, 8%, 9%, 10%, 15%, or 20% above or below the levels of reticulocytes in the subject prior to said treating.

62. The method of any one of claims 1 to 61, wherein the levels of reticulocytes in the subject remain in the range equal to or about 0.1%, 0.5%, 1%, 2%, 3%, 4%, 5%, 6%, 7%, 8%, 9%, 10%, 15%, or 20% above or below the levels of reticulocytes in a reference population.

63. The method of any one of claims 1 to 62, wherein the levels of white blood cells in the subject remain in the range equal to or about 0.1%, 0.5%, 1%, 2%, 3%, 4%, 5%, 6%, 7%, 8%, 9%, 10%, 15%, or 20% above or below the levels of white blood cells in the subject prior to said treating.


SEQUENCE LISTING (34 sequences)

SEQ ID NO: 1 - protein, 106 aa, Artificial Sequence
  human ActRIIB soluble (extracellular processed polypeptide with the N-terminal 6 aa of the EC domain deleted and the C-terminal 4 aa of the EC domain deleted (aa 25-130 of SEQ ID NO14) and with an L79D mutation
ETRECIYYNANWELERTNQSGLERCEGEQDKRLHCYASWRNSSGTIELVKKGCWDDDFNCYDRQECVATEENPQVYFCCCEGNFCNERFTHLPEAGGPEVTYEPPP

SEQ ID NO: 2 - protein, 512 aa, Homo sapiens
  human ActRIIB precursor protein sequence (A64)
MTAPWVALALLWGSLWPGSGRGEAETRECIYYNANWELERTNQSGLERCEGEQDKRLHCYASWANSSGTIELVKKGCWLDDFNCYDRQECVATEENPQVYFCCCEGNFCNERFTHLPEAGGPEVTYEPPPTAPTLLTVLAYSLLPIGGLSLIVLLAFWMYRHRKPPYGHVDIHEDPGPPPPSPLVGLKPLQLLEIKARGRFGCVWKAQLMNDFVAVKIFPLQDKQSWQSEREIFSTPGMKHENLLQFIAAEKRGSNLEVELWLITAFHDKGSLTDYLKGNIITWNELCHVAETMSRGLSYLHEDVPWCRGEGHKPSIAHRDFKSKNVLLKSDLTAVLADFGLAVRFEPGKPPGDTHGQVGTRRYMAPEVLEGAINFQRDAFLRIDMYAMGLVLWELVSRCKAADGPVDEYMLPFEEEIGQHPSLEELQEVVVHKKMRPTIKDHWLKHPGLAQLCVTIEECWDHDAEARLSAGCVEERVSLIRRSVNGTTSDCLVSLVTSVTNVDLPPKESSI

SEQ ID NO: 3 - protein, 116 aa, Homo sapiens
  human ActRIIB soluble (extracellular), processed polypeptide sequence (amino acids 19-134 of SEQ ID NO2)
SGRGEAETRECIYYNANWELERTNQSGLERCEGEQDKRLHCYASWANSSGTIELVKKGCWLDDFNCYDRQECVATEENPQVYFCCCEGNFCNERFTHLPEAGGPEVTYEPPPTAPT

SEQ ID NO: 4 - protein, 101 aa, Artificial Sequence
  human ActRIIB soluble (extracellular) processed polypeptide with the C-terminal 15 aa deleted (aa 19-119 of SEQ ID NO.2)
SGRGEAETRECIYYNANWELERTNQSGLERCEGEQDKRLHCYASWANSSGTIELVKKGCWLDDFNCYDRQECVATEENPQVYFCCCEGNFCNERFTHLPEA

SEQ ID NO: 5 - DNA, 1539 bp, Homo sapiens
  nucleic acid sequence encoding a human ActRIIB (A64) precursor protein
atgacggcgccctgggtggccctcgccctcctctggggatcgctgtggcccggctctgggcgtggggaggctgagacacgggagtgcatctactacaacgccaactgggagctggagcgcaccaaccagagcggcctggagcgctgcgaaggcgagcaggacaagcggctgcactgctacgcctcctgggccaacagctctggcaccatcgagctcgtgaagaagggctgctggctagatgacttcaactgctacgataggcaggagtgtgtggccactgaggagaacccccaggtgtacttctgctgctgtgaaggcaacttctgcaacgagcgcttcactcatttgccagaggctgggggcccggaagtcacgtacgagccacccccgacagcccccaccctgctcacggtgctggcctactcactgctgcccatcgggggcctttccctcatcgtcctgctggccttttggatgtaccggcatcgcaagcccccctacggtcatgtggacatccatgaggaccctgggcctccaccaccatcccctctggtgggcctgaagccactgcagctgctggagatcaaggctcgggggcgctttggctgtgtctggaaggcccagctcatgaatgactttgtagctgtcaagatcttcccactccaggacaagcagtcgtggcagagtgaacgggagatcttcagcacacctggcatgaagcacgagaacctgctacagttcattgctgccgagaagcgaggctccaacctcgaagtagagctgtggctcatcacggccttccatgacaagggctccctcacggattacctcaaggggaacatcatcacatggaacgaactgtgtcatgtagcagagacgatgtcacgaggcctctcatacctgcatgaggatgtgccctggtgccgtggcgagggccacaagccgtctattgcccacagggactttaaaagtaagaatgtattgctgaagagcgacctcacagccgtgctggctgactttggcttggctgttcgatttgagccagggaaacctccaggggacacccacggacaggtaggcacgagacggtacatggctcctgaggtgctcgagggagccatcaacttccagagagatgccttcctgcgcattgacatgtatgccatggggttggtgctgtgggagcttgtgtctcgctgcaaggctgcagacggacccgtggatgagtacatgctgccctttgaggaagagattggccagcacccttcgttggaggagctgcaggaggtggtggtgcacaagaagatgaggcccaccattaaagatcactggttgaaacacccgggcctggcccagctttgtgtgaccatcgaggagtgctgggaccatgatgcagaggctcgcttgtccgcgggctgtgtggaggagcgggtgtccctgattcggaggtcggtcaacggcactacctcggactgtctcgtttccctggtgacctctgtcaccaatgtggacctgccccctaaagagtcaagcatctaa

SEQ ID NO: 6 - protein, 344 aa, Artificial Sequence
  fusion protein comprising a soluble extracellular domain of ActRIIB (A64; SEQ ID NO 3) fused to an Fc domain
SGRGEAETRECIYYNANWELERTNQSGLERCEGEQDKRLHCYASWANSSGTIELVKKGCWLDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 329 aa, Artificial Sequence
  fusion protein comprising a soluble extracellular domain of ActRIIB (A64) with the C-terminal 15 amino acids deleted (SEQ ID NO4) fused to an Fc domain
SGRGEAETRECIYYNANWELERTNQSGLERCEGEQDKRLHCYASWANSSGTIELVKKGCWLDDFNCYDRQECVATEENPQVYFCCCEGNFCNERFTHLPEAGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 105 aa, Artificial Sequence
  human ActRIIB soluble (extracellular) processed polypeptide with the N-terminal 6 aa of the EC domain deleted and the C-terminal 5 aa of the EC domain deleted (aa 25-129 of SEQ ID NO14) and with an L79D mutation
ETRECIYYNANWELERTNQSGLERCEGEQDKRLHCYASWRNSSGTIELVKKGCWDDDFNCYDRQECVATEENPQVYFCCCEGNFCNERFTHLPEAGGPEVTYEPP

SEQ ID NO: 9 - protein, 107 aa, Artificial Sequence
  human ActRIIB soluble (extracellular) processed polypeptide with the N-terminal 6 aa of the EC domain deleted and the C-terminal 3 aa of the EC domain deleted (aa 25-131 of SEQ ID NO14) and with an L79D mutation
ETRECIYYNANWELERTNQSGLERCEGEQDKRLHCYASWRNSSGTIELVKKGCWDDDFNCYDRQECVATEENPQVYFCCCEGNFCNERFTHLPEAGGPEVTYEPPPT

SEQ ID NO: 10 - protein, 360 aa, Artificial Sequence
  Unprocessed ActRIIB-Fc fusion protein with the N-terminal 6 aa of the EC domain deleted and the C-terminal 3 aa of the EC domain deleted (aa 25-131 of SEQ ID NO14) and with an L79D mutation and with TPA leader sequence
MDAMKRGLCCVLLLCGAVFVSPGAAETRECIYYNANWELERTNQSGLERCEGEQDKRLHCYASWRNSSGTIELVKKGCWDDDFNCYDRQECVATEENPQVYFCCCEGNFCNERFTHLPEAGGPEVTYEPPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 335 aa, Artificial Sequence
  Processed ActRIIB-Fc fusion protein with the N-terminal 6 aa of the EC domain deleted and the C-terminal 3 aa of the EC domain deleted (aa 25-131 of SEQ ID NO14) and with an L79D mutation (Luspatercept)
ETRECIYYNANWELERTNQSGLERCEGEQDKRLHCYASWRNSSGTIELVKKGCWDDDFNCYDRQECVATEENPQVYFCCCEGNFCNERFTHLPEAGGPEVTYEPPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 115 aa, Homo sapiens
  human ActRIIB soluble (extracellular), processed polypeptide sequence (amino acids 20-134 of SEQ ID NO2)
GRGEAETRECIYYNANWELERTNQSGLERCEGEQDKRLHCYASWANSSGTIELVKKGCWLDDFNCYDRQECVATEENPQVYFCCCEGNFCNERFTHLPEAGGPEVTYEPPPTAPT

SEQ ID NO: 13 - protein, 100 aa, Artificial Sequence
  human ActRIIB soluble (extracellular), processed polypeptide sequence with the C-terminal 15 aa deleted (aa 20-119 of SEQ ID NO2)
GRGEAETRECIYYNANWELERTNQSGLERCEGEQDKRLHCYASWANSSGTIELVKKGCWLDDFNCYDRQECVATEENPQVYFCCCEGNFCNERFTHLPEA

SEQ ID NO: 14 - protein, 512 aa, Homo sapiens
  human ActRIIB precursor protein sequence (R64)
MTAPWVALALLWGSLWPGSGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTLLTVLAYSLLPIGGLSLIVLLAFWMYRHRKPPYGHVDIHEDPGPPPPSPLVGLKPLQLLEIKARGRFGCVWKAQLMNDFVAVKIFPLQDKQSWQSEREIFSTPGMKHENLLQFIAAEKRGSNLEVELWLITAFHDKGSLTDYLKGNIITWNELCHVAETMSRGLSYLHEDVPWCRGEGHKPSIAHRDFKSKNVLLKSDLTAVLADFGLAVRFEPGKPPGDTHGQVGTRRYMAPEVLEGAINFQRDAFLRIDMYAMGLVLWELVSRCKAADGPVDEYMLPFEEEIGQHPSLEELQEVVVHKKMRPTIKDHWLKHPGLAQLCVTIEECWDHDAEARLSAGCVEERVSLIRRSVNGTTSDCLVSLVTSVTNVDLPPKESSI

SEQ ID NO: 15 - protein, 116 aa, Artificial Sequence
  human ActRIIB soluble (extracellular), processed polypeptide sequence (amino acids 19-134 of SEQ ID NO14)
SGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPT

SEQ ID NO: 16 - protein, 101 aa, Artificial Sequence
  human ActRIIB soluble (extracellular), processed polypeptide sequence with the C-terminal 15 aa deleted (aa 19-119 of SEQ ID NO14)
SGRGEAETRECIYYNANWELERTNQSGLERCEGEQDKRLHCYASWRNSSGTIELVKKGCWLDDFNCYDRQECVATEENPQVYFCCCEGNFCNERFTHLPEA

SEQ ID NO: 17 - protein, 115 aa, Artificial Sequence
  human ActRIIB soluble (extracellular), processed polypeptide sequence (amino acids 20-134 of SEQ ID NO14)
GRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPT

SEQ ID NO: 18 - protein, 100 aa, Artificial Sequence
  human ActRIIB soluble (extracellular), processed polypeptide sequence with the C-terminal 15 aa deleted (aa 20-119 of SEQ ID NO14)
GRGEAETRECIYYNANWELERTNQSGLERCEGEQDKRLHCYASWRNSSGTIELVKKGCWLDDFNCYDRQECVATEENPQVYFCCCEGNFCNERFTHLPEA

SEQ ID NO: 19 - protein, 107 aa, Artificial Sequence
  human ActRIIB soluble (extracellular) processed polypeptide with the N-terminal 6 aa of the EC domain deleted and the C-terminal 3 aa of the EC domain deleted (aa 25-131 of SEQ ID NO2) and with an L79D mutation
ETRECIYYNANWELERTNQSGLERCEGEQDKRLHCYASWANSSGTIELVKKGCWDDDFNCYDRQECVATEENPQVYFCCCEGNFCNERFTHLPEAGGPEVTYEPPPT

SEQ ID NO: 20 - protein, 360 aa, Artificial Sequence
  Unprocessed ActRIIB-Fc fusion protein with the N-terminal 6 aa of the EC domain deleted and the C-terminal 3 aa of the EC domain deleted (aa 25-131 of SEQ ID NO2) and with an L79D mutation and with TPA leader sequence
MDAMKRGLCCVLLLCGAVFVSPGAAETRECIYYNANWELERTNQSGLERCEGEQDKRLHCYASWANSSGTIELVKKGCWDDDFNCYDRQECVATEENPQVYFCCCEGNFCNERFTHLPEAGGPEVTYEPPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 335 aa, Artificial Sequence
  Processed ActRIIB-Fc fusion protein with the N-terminal 6 aa of the EC domain deleted and the C-terminal 3 aa of the EC domain deleted (aa 25-131 of SEQ ID NO2) and with an L79D mutation
ETRECIYYNANWELERTNQSGLERCEGEQDKRLHCYASWANSSGTIELVKKGCWDDDFNCYDRQECVATEENPQVYFCCCEGNFCNERFTHLPEAGGPEVTYEPPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 115 aa, Artificial Sequence
  human ActRIIB soluble (extracellular), processed polypeptide sequence (amino acids 20-134 of SEQ ID NO14) with L79D mutation
GRGEAETRECIYYNANWELERTNQSGLERCEGEQDKRLHCYASWRNSSGTIELVKKGCWDDDFNCYDRQECVATEENPQVYFCCCEGNFCNERFTHLPEAGGPEVTYEPPPTAPT

SEQ ID NO: 23 - protein, 115 aa, Artificial Sequence
  human ActRIIB soluble (extracellular), processed polypeptide sequence (amino acids 20-134 of SEQ ID NO2) with L79D mutation
GRGEAETRECIYYNANWELERTNQSGLERCEGEQDKRLHCYASWANSSGTIELVKKGCWDDDFNCYDRQECVATEENPQVYFCCCEGNFCNERFTHLPEAGGPEVTYEPPPTAPT

SEQ ID NO: 24 - protein, 343 aa, Artificial Sequence
  human ActRIIB soluble (extracellular), processed polypeptide sequence (aa 20-134 of SEQ ID NO14) with L79D mutation fused to an Fc domain with a GGG linker
GRGEAETRECIYYNANWELERTNQSGLERCEGEQDKRLHCYASWRNSSGTIELVKKGCWDDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 343 aa, Artificial Sequence
  human ActRIIB soluble (extracellular), processed polypeptide sequence (aa 20-134 of SEQ ID NO2) with L79D mutation fused to an Fc domain
GRGEAETRECIYYNANWELERTNQSGLERCEGEQDKRLHCYASWANSSGTIELVKKGCWDDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 368 aa, Artificial Sequence
  human ActRIIB soluble (extracellular), processed polypeptide sequence (aa 20-134 of SEQ ID NO14) with L79D mutation fused to an Fc domain and with TPA leader sequence
MDAMKRGLCCVLLLCGAVFVSPGASGRGEAETRECIYYNANWELERTNQSGLERCEGEQDKRLHCYASWRNSSGTIELVKKGCWDDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 368 aa, Artificial Sequence
  human ActRIIB soluble (extracellular), processed polypeptide sequence (aa 20-134 of SEQ ID NO2) with L79D mutation fused to an Fc domain and with TPA leader sequence
MDAMKRGLCCVLLLCGAVFVSPGASGRGEAETRECIYYNANWELERTNQSGLERCEGEQDKRLHCYASWANSSGTIELVKKGCWDDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 141 aa, Artificial Sequence
  human ActRIIB soluble (extracellular), processed polypeptide sequence having a variant C-terminal sequence (disclosed in WO2007/053775)
GRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEGPWASTTIPSGGPEATAAAGDQGSGALWLCLEGPAHE

SEQ ID NO: 29 - protein, 141 aa, Artificial Sequence
  human ActRIIB soluble (extracellular), processed polypeptide sequence having a variant C-terminal sequence (disclosed in WO2007/053775) having an L79D mutation
GRGEAETRECIYYNANWELERTNQSGLERCEGEQDKRLHCYASWRNSSGTIELVKKGCWDDDFNCYDRQECVATEENPQVYFCCCEGNFCNERFTHLPEAGGPEGPWASTTIPSGGPEATAAAGDQGSGALWLCLEGPAHE

SEQ ID NO: 30 - protein, 370 aa, Artificial Sequence
  human ActRIIB soluble (extracellular), processed polypeptide sequence having a variant C-terminal sequence (disclosed in WO2007/053775) having an L79D mutation fused to an Fc domain with a TGGG linker
GRGEAETRECIYYNANWELERTNQSGLERCEGEQDKRLHCYASWRNSSGTIELVKKGCWDDDFNCYDRQECVATEENPQVYFCCCEGNFCNERFTHLPEAGGPEGPWASTTIPSGGPEATAAAGDQGSGALWLCLEGPAHETGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - DNA, 1083 bp, Artificial Sequence
  Nucleic Acid Sequence Encoding SEQ ID NO10
atggatgcaatgaagagagggctctgctgtgtgctgctgctgtgtggagcagtcttcgtttcgcccggcgccgccgaaacccgcgaatgtatttattacaatgctaattgggaactcgaacggacgaaccaatccgggctcgaacggtgtgagggggaacaggataaacgcctccattgctatgcgtcgtggaggaactcctccgggacgattgaactggtcaagaaagggtgctgggacgacgatttcaattgttatgaccgccaggaatgtgtcgcgaccgaagagaatccgcaggtctatttctgttgttgcgaggggaatttctgtaatgaacggtttacccacctccccgaagccggcgggcccgaggtgacctatgaacccccgcccaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 32 - protein, 344 aa, Artificial Sequence
  fusion protein comprising a soluble extracellular domain of ActRIIB (R64; SEQ ID NO15) fused to an Fc domain
SGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 329 aa, Artificial Sequence
  fusion protein comprising a soluble extracellular domain of ActRIIB (R64) with the C-terminal 15 aa deleted (SEQ ID NO16) fused to an Fc domain
SGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - DNA, 1083 bp, Artificial Sequence
  misc_signal (1)..(72): nt sequence encoding TPA signal peptide
  Nucleic acid sequence that encodes SEQ ID NO1 and tissue plasminogen activator (TPA signal peptide sequence)
atggatgcaatgaagagagggctctgctgtgtgctgctgctgtgtggagcagtcttcgtttcgcccggcgccgccgaaacccgcgaatgtatttattacaatgctaattgggaactcgaacggacgaaccaatccgggctcgaacggtgtgagggggaacaggataaacgcctccattgctatgcgtcgtggaggaactcctccgggacgattgaactggtcaagaaagggtgctgggacgacgatttcaattgttatgaccgccaggaatgtgtcgcgaccgaagagaatccgcaggtctatttctgttgttgcgaggggaatttctgtaatgaacggtttacccacctccccgaagccggcgggcccgaggtgacctatgaacccccgcccaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga